SEQUENCE LISTING (156 sequences)

SEQ ID NO: 1 - DNA, 83 bp, synthetic construct
  source 1..83: mol_type = other DNA; note = AKMmono01v2
cccactccctggttttttaaaactcagagnnnncttcagcttcccgatatccgacggtagtgtnnnnactgaggtagtgtctg

SEQ ID NO: 2 - DNA, 83 bp, synthetic construct
  source 1..83: mol_type = other DNA; note = AKMmono02
gaggagtttcttaagaatattttctcttnnnncttcagcttcccgatatccgacggtagtgtnnnncatgcttgttttccttt

SEQ ID NO: 3 - DNA, 83 bp, synthetic construct
  source 1..83: mol_type = other DNA; note = AKMmono03
tgggcatcaatccttccaagtcttcnnnncttcagcttcccgatatccgacggtagtgtnnnnggcattcaagtctaacagag

SEQ ID NO: 4 - DNA, 83 bp, synthetic construct
  source 1..83: mol_type = other DNA; note = AKMmono04
gtgtgtttaattgctttaccttagggtgnnnncttcagcttcccgatatccgacggtagtgtnnnngacactgaccaaattgt

SEQ ID NO: 5 - DNA, 83 bp, synthetic construct
  source 1..83: mol_type = other DNA; note = AKMmono05
gcttatcaaaagatatatgaggattcctgnnnncttcagcttcccgatatccgacggtagtgtnnnngttcccaaaaggatag

SEQ ID NO: 6 - DNA, 83 bp, synthetic construct
  source 1..83: mol_type = other DNA; note = AKMmono06
tctatctctattggataatcttcaaagacnnnncttcagcttcccgatatccgacggtagtgtnnnncctttctactacatgc

SEQ ID NO: 7 - DNA, 83 bp, synthetic construct
  source 1..83: mol_type = other DNA; note = AKMmono07
taaaatctaagtgccccaatcatctgtcnnnncttcagcttcccgatatccgacggtagtgtnnnngccttttccctaaggtg

SEQ ID NO: 8 - DNA, 83 bp, synthetic construct
  source 1..83: mol_type = other DNA; note = AKMmono08v2
atccttcatagttttatcagtcactannnncttcagcttcccgatatccgacggtagtgtnnnnctttgagatttgtcttatc

SEQ ID NO: 9 - DNA, 83 bp, synthetic construct
  source 1..83: mol_type = other DNA; note = AKMmono10v2
cattcttacctttatcacgatcatagagcnnnncttcagcttcccgatatccgacggtagtgtnnnnaccaatctttgacaat

SEQ ID NO: 10 - DNA, 83 bp, synthetic construct
  source 1..83: mol_type = other DNA; note = AKMmono11
gtcattaacataatgcctccttatgttcnnnncttcagcttcccgatatccgacggtagtgtnnnngtcttgcacatgtaccc

SEQ ID NO: 11 - DNA, 83 bp, synthetic construct
  source 1..83: mol_type = other DNA; note = AKMmono12
cagtgtaatttgaaaactccagctgtccannnncttcagcttcccgatatccgacggtagtgtnnnnctgtttatgcagcaag

SEQ ID NO: 12 - DNA, 83 bp, synthetic construct
  source 1..83: mol_type = other DNA; note = AKMmono13
ttctcttatggatgttgtgactttaatcnnnncttcagcttcccgatatccgacggtagtgtnnnncagtttccaccctcagt

SEQ ID NO: 13 - DNA, 83 bp, synthetic construct
  source 1..83: mol_type = other DNA; note = AKMmono14
gctaaaggaggaggtggattttttctctnnnncttcagcttcccgatatccgacggtagtgtnnnnctaaaatagacttggaa

SEQ ID NO: 14 - DNA, 83 bp, synthetic construct
  source 1..83: mol_type = other DNA; note = AKMmono16
cctcagtgagaccaatagagcttgttcagnnnncttcagcttcccgatatccgacggtagtgtnnnngctccttcacacttgt

SEQ ID NO: 15 - DNA, 81 bp, synthetic construct
  source 1..81: mol_type = other DNA; note = AKMmono17v2
gcctctacgtggatgttttatgnnnncttcagcttcccgatatccgacggtagtgtnnnntcaacttttaaatcaaattag

SEQ ID NO: 16 - DNA, 83 bp, synthetic construct
  source 1..83: mol_type = other DNA; note = AKMmono22
ttcctattcatgtgatgagatgactacannnncttcagcttcccgatatccgacggtagtgtnnnngttggactttgccccag

SEQ ID NO: 17 - DNA, 83 bp, synthetic construct
  source 1..83: mol_type = other DNA; note = EJmono01
cagtaatttcagagatatatagagaaaacnnnncttcagcttcccgatatccgacggtagtgtnnnnagacatcaatagcttt

SEQ ID NO: 18 - DNA, 83 bp, synthetic construct
  source 1..83: mol_type = other DNA; note = EJmono02
accccatcaccaaagacaccnnnncttcagcttcccgatatccgacggtagtgtnnnnggctagcctggctttgagctttacc

SEQ ID NO: 19 - DNA, 83 bp, synthetic construct
  source 1..83: mol_type = other DNA; note = EJmono03
gcatttgctgcctgattctcctcatgtggnnnncttcagcttcccgatatccgacggtagtgtnnnngcctaggcactgtctc

SEQ ID NO: 20 - DNA, 83 bp, synthetic construct
  source 1..83: mol_type = other DNA; note = EJmono04
ccattcacaaataaaattacccacacagnnnncttcagcttcccgatatccgacggtagtgtnnnngctctaaccaaatccat

SEQ ID NO: 21 - DNA, 83 bp, synthetic construct
  source 1..83: mol_type = other DNA; note = EJmono05
gagtcacattcaggccttaaaataactgtnnnncttcagcttcccgatatccgacggtagtgtnnnnggtttcactgctgctt

SEQ ID NO: 22 - DNA, 83 bp, synthetic construct
  source 1..83: mol_type = other DNA; note = EJmono06v2
cactaatgaaagggcaatcgctgtaggnnnncttcagcttcccgatatccgacggtagtgtnnnngcctcaagggttaggctg

SEQ ID NO: 23 - DNA, 83 bp, synthetic construct
  source 1..83: mol_type = other DNA; note = EJmono12
gctagtaaggtgggaatttgtaaacctgtnnnncttcagcttcccgatatccgacggtagtgtnnnnaagatgtggtcaactt

SEQ ID NO: 24 - DNA, 81 bp, synthetic construct
  source 1..81: mol_type = other DNA; note = EJmono13v2
cggtctttccacataggataattggggnnnncttcagcttcccgatatccgacggtagtgtnnnntgaacgcaagtggcaa

SEQ ID NO: 25 - DNA, 83 bp, synthetic construct
  source 1..83: mol_type = other DNA; note = EJmono14v2
aaagtgcaagtaaatattaaacaactggnnnncttcagcttcccgatatccgacggtagtgtnnnncctcagttccttcttta

SEQ ID NO: 26 - DNA, 83 bp, synthetic construct
  source 1..83: mol_type = other DNA; note = EJmono16
accaaccaacaaacaaaaagctgaaaccnnnncttcagcttcccgatatccgacggtagtgtnnnngtgggtgattgcaattg

SEQ ID NO: 27 - DNA, 83 bp, synthetic construct
  source 1..83: mol_type = other DNA; note = EJmono21v2
gcttataaaaacctcagctaggtctcagannnncttcagcttcccgatatccgacggtagtgtnnnntatagtgcagtttggt

SEQ ID NO: 28 - DNA, 83 bp, synthetic construct
  source 1..83: mol_type = other DNA; note = HGtetra23ms2
ctttgtacttgtatctctggatgccnnnncttcagcttcccgatatccgacggtagtgtnnnnggacaagagtgaagcttcat

SEQ ID NO: 29 - DNA, 83 bp, synthetic construct
  source 1..83: mol_type = other DNA; note = LMmono01
ctatgggattatgtagaaagactgaaccnnnncttcagcttcccgatatccgacggtagtgtnnnnctgaaataagatacact

SEQ ID NO: 30 - DNA, 83 bp, synthetic construct
  source 1..83: mol_type = other DNA; note = LMmono03
taatagagttgaccatcacaacgaatggnnnncttcagcttcccgatatccgacggtagtgtnnnngttagtatcactagggc

SEQ ID NO: 31 - DNA, 82 bp, synthetic construct
  source 1..82: mol_type = other DNA; note = LMmono04v2
tcttcattccacgtaaccnnnncttcagcttcccgatatccgacggtagtgtnnnntgccatgttccaatcaattctaaatc

SEQ ID NO: 32 - DNA, 80 bp, synthetic construct
  source 1..80: mol_type = other DNA; note = LMmono05v2
gagtacttacgatgtgccaaatacnnnncttcagcttcccgatatccgacggtagtgtnnnnaggcacaaaaagataaaa

SEQ ID NO: 33 - DNA, 83 bp, synthetic construct
  source 1..83: mol_type = other DNA; note = LMmono07
cccagtctcaactattatgtaatagcagnnnncttcagcttcccgatatccgacggtagtgtnnnngctagctccttgatctt

SEQ ID NO: 34 - DNA, 83 bp, synthetic construct
  source 1..83: mol_type = other DNA; note = LMmono08
tggctggaaattttccaaacttgatgnnnncttcagcttcccgatatccgacggtagtgtnnnnaagaagcccaatacacagg

SEQ ID NO: 35 - DNA, 83 bp, synthetic construct
  source 1..83: mol_type = other DNA; note = LMmono09
gttgacctcgaactccagtccnnnncttcagcttcccgatatccgacggtagtgtnnnngcgtcctccctttatgttttgttg

SEQ ID NO: 36 - DNA, 82 bp, synthetic construct
  source 1..82: mol_type = other DNA; note = LMmono10v2
gaagtgtgggaaaactggcactctggcnnnncttcagcttcccgatatccgacggtagtgtnnnngaaagcaggagactacg

SEQ ID NO: 37 - DNA, 83 bp, synthetic construct
  source 1..83: mol_type = other DNA; note = LMmono12
tccacagtgacacaaaccaattccnnnncttcagcttcccgatatccgacggtagtgtnnnngatgcactttgaaacagcctt

SEQ ID NO: 38 - DNA, 83 bp, synthetic construct
  source 1..83: mol_type = other DNA; note = LMmono16
catccttaagagagacaaacctctgnnnncttcagcttcccgatatccgacggtagtgtnnnngggactttccaggatttgcc

SEQ ID NO: 39 - DNA, 83 bp, synthetic construct
  source 1..83: mol_type = other DNA; note = MSJcom06ms1 + ms2
tgggtgcggtggcccacacctgtaattnnnncttcagcttcccgatatccgacggtagtgtnnnncctcactgagtagttttt

SEQ ID NO: 40 - DNA, 83 bp, synthetic construct
  source 1..83: mol_type = other DNA; note = MSJmono10
gtacatcaatttggggagaatttgcatccnnnncttcagcttcccgatatccgacggtagtgtnnnnaaacaaccttgtctgt

SEQ ID NO: 41 - DNA, 83 bp, synthetic construct
  source 1..83: mol_type = other DNA; note = MSJmono11
tcccccttctctctctttctcctgctannnncttcagcttcccgatatccgacggtagtgtnnnnagccgagatcacacctgg

SEQ ID NO: 42 - DNA, 83 bp, synthetic construct
  source 1..83: mol_type = other DNA; note = MSJmono15
gtgaagcctgaccaatgaagacatcnnnncttcagcttcccgatatccgacggtagtgtnnnngggcaacagagagagatgcc

SEQ ID NO: 43 - DNA, 83 bp, synthetic construct
  source 1..83: mol_type = other DNA; note = MSJmono17
gtgggtgtactaaacatatttgatacctnnnncttcagcttcccgatatccgacggtagtgtnnnntagcttgggtgacggag

SEQ ID NO: 44 - DNA, 83 bp, synthetic construct
  source 1..83: mol_type = other DNA; note = MSJmono19ms1 + ms2
cacaaattggtaacactgatccatctnnnncttcagcttcccgatatccgacggtagtgtnnnngagagattctgtctctacc

SEQ ID NO: 45 - DNA, 83 bp, synthetic construct
  source 1..83: mol_type = other DNA; note = MSJmono20
gacttgaggatatcctccaggaaaatgnnnncttcagcttcccgatatccgacggtagtgtnnnntagcactgcagtgagctg

SEQ ID NO: 46 - DNA, 83 bp, synthetic construct
  source 1..83: mol_type = other DNA; note = MSJmono22
gtgtttcagatacgtcggtaacnnnncttcagcttcccgatatccgacggtagtgtnnnncgtgccattgcactctatcctgg

SEQ ID NO: 47 - DNA, 83 bp, synthetic construct
  source 1..83: mol_type = other DNA; note = MSJmono23v2
ttcccacctcagcctcctgagtagctagcnnnncttcagcttcccgatatccgacggtagtgtnnnncacatcctacactcca

SEQ ID NO: 48 - DNA, 83 bp, synthetic construct
  source 1..83: mol_type = other DNA; note = MSJmono26
gcggagtctcgctctgtcgcccatgctggnnnncttcagcttcccgatatccgacggtagtgtnnnnatacccacatgatcat

SEQ ID NO: 49 - DNA, 83 bp, synthetic construct
  source 1..83: mol_type = other DNA; note = MSJmono27
tggtgggattgttcacacctgtaatcccnnnncttcagcttcccgatatccgacggtagtgtnnnnactgtaacctggccaac

SEQ ID NO: 50 - DNA, 83 bp, synthetic construct
  source 1..83: mol_type = other DNA; note = MSJmono30v2
tcctttataaattacccagtctcggccnnnncttcagcttcccgatatccgacggtagtgtnnnnaaataaagtggttaagaa

SEQ ID NO: 51 - DNA, 83 bp, synthetic construct
  source 1..83: mol_type = other DNA; note = MSJmono32
ggagttgcaaccctggttttagatnnnncttcagcttcccgatatccgacggtagtgtnnnnaatcggtgtgctgtattcagg

SEQ ID NO: 52 - DNA, 83 bp, synthetic construct
  source 1..83: mol_type = other DNA; note = MSJmono36
tggcagaagtccttccttatcagtannnncttcagcttcccgatatccgacggtagtgtnnnngtctgggtgagacagtgacc

SEQ ID NO: 53 - DNA, 83 bp, synthetic construct
  source 1..83: mol_type = other DNA; note = MSJmono37
gcctgtagtgtagtggcgtggtcatagnnnncttcagcttcccgatatccgacggtagtgtnnnnattgggagtgaagagaaa

SEQ ID NO: 54 - DNA, 83 bp, synthetic construct
  source 1..83: mol_type = other DNA; note = MSJmono38
gctgggccaggacactcagagttctctctnnnncttcagcttcccgatatccgacggtagtgtnnnntcatcatattctggtg

SEQ ID NO: 55 - DNA, 83 bp, synthetic construct
  source 1..83: mol_type = other DNA; note = MSJmono39
ccagtaaatacgatacatggtttgtaggnnnncttcagcttcccgatatccgacggtagtgtnnnnagctagataaaacacac

SEQ ID NO: 56 - DNA, 83 bp, synthetic construct
  source 1..83: mol_type = other DNA; note = MSJmono40
cagattgaggtactgaatagttttgnnnncttcagcttcccgatatccgacggtagtgtnnnntcagcctgggcaacagaccg

SEQ ID NO: 57 - DNA, 83 bp, synthetic construct
  source 1..83: mol_type = other DNA; note = MSJmono41
gttcatccccatttcccannnncttcagcttcccgatatccgacggtagtgtnnnnagcctgggtgacagagtgtgagaccct

SEQ ID NO: 58 - DNA, 83 bp, synthetic construct
  source 1..83: mol_type = other DNA; note = MSJmono44
ttcacaggacccatgctcagcccnnnncttcagcttcccgatatccgacggtagtgtnnnntgtactccagcctggacgagtg

SEQ ID NO: 59 - DNA, 83 bp, synthetic construct
  source 1..83: mol_type = other DNA; note = MSJmono45
ttcctggcgtgtcctgtgctgctccagcnnnncttcagcttcccgatatccgacggtagtgtnnnngcctggtgacataggaa

SEQ ID NO: 60 - DNA, 82 bp, synthetic construct
  source 1..82: mol_type = other DNA; note = MSJmono46
gataaatggagcttcaggtatggcatgnnnncttcagcttcccgatatccgacggtagtgtnnnnacataaatctaaaacca

SEQ ID NO: 61 - DNA, 80 bp, synthetic construct
  source 1..80: mol_type = other DNA; note = AKMmono02_SR_SIxxxF
caagcagaagacggcatacgagatnnnnnnnnacacgcacgatccgacggtagtgtaactcctctaaggaatatgctctc

SEQ ID NO: 62 - DNA, 81 bp, synthetic construct
  source 1..81: mol_type = other DNA; note = AKMmono02_SR_UR
aatgatacggcgaccaccgagatctacacatacgagatccgtaatcgggaagctgaagacacactagttgcatacatgctt

SEQ ID NO: 63 - DNA, 80 bp, synthetic construct
  source 1..80: mol_type = other DNA; note = AKMmono03_SR_SIxxxF
caagcagaagacggcatacgagatnnnnnnnnacacgcacgatccgacggtagtgtggcattcaagtctaacagagctta

SEQ ID NO: 64 - DNA, 79 bp, synthetic construct
  source 1..79: mol_type = other DNA; note = AKMmono03_SR_UR
aatgatacggcgaccaccgagatctacacatacgagatccgtaatcgggaagctgaagtgatgcccatctttcgtactt

SEQ ID NO: 65 - DNA, 80 bp, synthetic construct
  source 1..80: mol_type = other DNA; note = AKMmono04_SR_SIxxxF
caagcagaagacggcatacgagatnnnnnnnnacacgcacgatccgacggtagtgttggacactgaccaaattgtcaaac

SEQ ID NO: 66 - DNA, 82 bp, synthetic construct
  source 1..82: mol_type = other DNA; note = AKMmono04_SR_UR
aatgatacggcgaccaccgagatctacacatacgagatccgtaatcgggaagctgaagaagcaattaaacacacttttggtg

SEQ ID NO: 67 - DNA, 81 bp, synthetic construct
  source 1..81: mol_type = other DNA; note = AKMmono12_SR_UR
aatgatacggcgaccaccgagatctacacatacgagatccgtaatcgggaagctgaaggctggagttttcaaattacactg

SEQ ID NO: 68 - DNA, 79 bp, synthetic construct
  source 1..79: mol_type = other DNA; note = AKMmono13_SR_SIxxxF
caagcagaagacggcatacgagatnnnnnnnnacacgcacgatccgacggtagtgtaataaaaaccaccacacagaagg

SEQ ID NO: 69 - DNA, 82 bp, synthetic construct
  source 1..82: mol_type = other DNA; note = AKMmono13_SR_UR
aatgatacggcgaccaccgagatctacacatacgagatccgtaatcgggaagctgaaggagcttgattaaagtcacaacatc

SEQ ID NO: 70 - DNA, 79 bp, synthetic construct
  source 1..79: mol_type = other DNA; note = AKMmono16_SR_SIxxxF
caagcagaagacggcatacgagatnnnnnnnnacacgcacgatccgacggtagtgtatgcatttgctccttcacacttg

SEQ ID NO: 71 - DNA, 81 bp, synthetic construct
  source 1..81: mol_type = other DNA; note = AKMmono16_SR_UR
aatgatacggcgaccaccgagatctacacatacgagatccgtaatcgggaagctgaagcactgaggcagtatctctgtcat

SEQ ID NO: 72 - DNA, 79 bp, synthetic construct
  source 1..79: mol_type = other DNA; note = EJmono12_SR_SIxxxF
caagcagaagacggcatacgagatnnnnnnnnacacgcacgatccgacggtagtgtacttgagaaagatgtggtcaact

SEQ ID NO: 73 - DNA, 80 bp, synthetic construct
  source 1..80: mol_type = other DNA; note = EJmono12_SR_UR
aatgatacggcgaccaccgagatctacacatacgagatccgtaatcgggaagctgaagacaggtttacaaattcccacct

SEQ ID NO: 74 - DNA, 83 bp, synthetic construct
  source 1..83: mol_type = other DNA; note = MSJmono20_SR_UR
aatgatacggcgaccaccgagatctacacatacgagatccgtaatcgggaagctgaagattttacttcctcattttcctggag

SEQ ID NO: 75 - DNA, 81 bp, synthetic construct
  source 1..81: mol_type = other DNA; note = MSJmono39_SR_SIxxxF
caagcagaagacggcatacgagatnnnnnnnnacacgcacgatccgacggtagtgtacttagctagataaaacacacattt

SEQ ID NO: 76 - DNA, 82 bp, synthetic construct
  source 1..82: mol_type = other DNA; note = MSJmono39_SR_UR
aatgatacggcgaccaccgagatctacacatacgagatccgtaatcgggaagctgaagccatgtatcgtatttactggtggt

SEQ ID NO: 77 - DNA, 76 bp, synthetic construct
  source 1..76: mol_type = other DNA; note = MSJmono45_SR_SIxxxF
caagcagaagacggcatacgagatnnnnnnnnacacgcacgatccgacggtagtgttcatgctactgcactccagc

SEQ ID NO: 78 - DNA, 77 bp, synthetic construct
  source 1..77: mol_type = other DNA; note = MSJmono45_SR_UR
aatgatacggcgaccaccgagatctacacatacgagatccgtaatcgggaagctgaagggaacagacgggccaaact

SEQ ID NO: 79 - DNA, 76 bp, synthetic construct
  source 1..76: mol_type = other DNA; note = GM07_SR_SIxxxF
caagcagaagacggcatacgagatnnnnnnnnacacgcacgatccgacggtagtgtgggcccttttaggcatatag

SEQ ID NO: 80 - DNA, 77 bp, synthetic construct
  source 1..77: mol_type = other DNA; note = GM07_SR_UR
aatgatacggcgaccaccgagatctacacatacgagatccgtaatcgggaagctgaagcatatggggtttggtcaca

SEQ ID NO: 81 - DNA, 74 bp, synthetic construct
  source 1..74: mol_type = other DNA; note = LR36_SR_SIxxxF
caagcagaagacggcatacgagatnnnnnnnnacacgcacgatccgacggtagtgtgctggcacttgtggtgac

SEQ ID NO: 82 - DNA, 77 bp, synthetic construct
  source 1..77: mol_type = other DNA; note = LR36_SR_UR
aatgatacggcgaccaccgagatctacacatacgagatccgtaatcgggaagctgaagtttgcactctttgcacacc

SEQ ID NO: 83 - DNA, 75 bp, synthetic construct
  source 1..75: mol_type = other DNA; note = LR44_SR_SIxxxF
caagcagaagacggcatacgagatnnnnnnnnacacgcacgatccgacggtagtgtaggctcatttgaggccaag

SEQ ID NO: 84 - DNA, 82 bp, synthetic construct
  source 1..82: mol_type = other DNA; note = LR44_SR_UR
aatgatacggcgaccaccgagatctacacatacgagatccgtaatcgggaagctgaagaaaaacagtcaaggaacaaaagtg

SEQ ID NO: 85 - DNA, 76 bp, synthetic construct
  source 1..76: mol_type = other DNA; note = BRAF_V600_SR_SIxxxF
caagcagaagacggcatacgagatnnnnnnnnacacgcacgatccgacggtagtgtctgttcaaactgatgggacc

SEQ ID NO: 86 - DNA, 81 bp, synthetic construct
  source 1..81: mol_type = other DNA; note = BRAF_V600_SR_UR
aatgatacggcgaccaccgagatctacacatacgagatccgtaatcgggaagctgaagcttcatgaagacctcacagtaaa

SEQ ID NO: 87 - DNA, 81 bp, synthetic construct
  source 1..81: mol_type = other DNA; note = KRAS_G12_SR_SIxxxF2
caagcagaagacggcatacgagatnnnnnnnnacacgcacgatccgacggtagtgtattataaggcctgctgaaaatgact

SEQ ID NO: 88 - DNA, 82 bp, synthetic construct
  source 1..82: mol_type = other DNA; note = KRAS_G12_SR_UR2
aatgatacggcgaccaccgagatctacacatacgagatccgtaatcgggaagctgaagtagctgtatcgtcaaggcactctt

SEQ ID NO: 89 - DNA, 78 bp, synthetic construct
  source 1..78: mol_type = other DNA; note = KRAS_Q61_SR_SIxxxF2
caagcagaagacggcatacgagatnnnnnnnnacacgcacgatccgacggtagtgtagtcctcatgtactggtccctc

SEQ ID NO: 90 - DNA, 82 bp, synthetic construct
  source 1..82: mol_type = other DNA; note = KRAS_Q61_SR_UR2
aatgatacggcgaccaccgagatctacacatacgagatccgtaatcgggaagctgaagacctgtctcttggatattctcgac

SEQ ID NO: 91 - DNA, 81 bp, synthetic construct
  source 1..81: mol_type = other DNA; note = KRAS_S145_SR_SIxxxF2
caagcagaagacggcatacgagatnnnnnnnnacacgcacgatccgacggtagtgtatttcagtgttacttacctgtcttg

SEQ ID NO: 92 - DNA, 78 bp, synthetic construct
  source 1..78: mol_type = other DNA; note = KRAS_S145_SR_UR2
aatgatacggcgaccaccgagatctacacatacgagatccgtaatcgggaagctgaagacaggctcaggacttagcaa

SEQ ID NO: 93 - DNA, 75 bp, synthetic construct
  source 1..75: mol_type = other DNA; note = NRAS_G12_SR_SIxxxF
caagcagaagacggcatacgagatnnnnnnnnacacgcacgatccgacggtagtgtacaggttcttgctggtgtg

SEQ ID NO: 94 - DNA, 77 bp, synthetic construct
  source 1..77: mol_type = other DNA; note = NRAS_G12_SR_UR
aatgatacggcgaccaccgagatctacacatacgagatccgtaatcgggaagctgaagactgggcctcacctctatg

SEQ ID NO: 95 - DNA, 78 bp, synthetic construct
  source 1..78: mol_type = other DNA; note = NRAS_Q61_SR_SIxxxF2
caagcagaagacggcatacgagatnnnnnnnnacacgcacgatccgacggtagtgttgtattggtctctcatggcact

SEQ ID NO: 96 - DNA, 82 bp, synthetic construct
  source 1..82: mol_type = other DNA; note = NRAS_Q61_SR_UR2
aatgatacggcgaccaccgagatctacacatacgagatccgtaatcgggaagctgaagagatggtgaaacctgtttgttgga

SEQ ID NO: 97 - DNA, 79 bp, synthetic construct
  source 1..79: mol_type = other DNA; note = NRAS_S145_SR_SIxxxF2
caagcagaagacggcatacgagatnnnnnnnnacacgcacgatccgacggtagtgtatgctgaaagctgtaccatacct

SEQ ID NO: 98 - DNA, 79 bp, synthetic construct
  source 1..79: mol_type = other DNA; note = NRAS_S145_SR_UR2
aatgatacggcgaccaccgagatctacacatacgagatccgtaatcgggaagctgaagacgaactggccaagagttacg

SEQ ID NO: 99 - DNA, 41 bp, synthetic construct
  source 1..41: mol_type = other DNA; note = EJ-M12_1F
tccgacggtagtgtnnnnacttgagaaagatgtggtcaact

SEQ ID NO: 100 - DNA, 40 bp, synthetic construct
  source 1..40: mol_type = other DNA; note = EJ-M12_1R
tcgggaagctgaagnnnnacaggtttacaaattcccacct

SEQ ID NO: 101 - DNA, 37 bp, synthetic construct
  source 1..37: mol_type = other DNA; note = MSJ-M20_1F
tccgacggtagtgtnnnnattgtactccagcacgggt

SEQ ID NO: 102 - DNA, 43 bp, synthetic construct
  source 1..43: mol_type = other DNA; note = MSJ-M20_1R
tcgggaagctgaagnnnnattttacttcctcattttcctggag

SEQ ID NO: 103 - DNA, 38 bp, synthetic construct
  source 1..38: mol_type = other DNA; note = AKM-M7_2F
tccgacggtagtgtnnnnaaggccttttccctaaggtg

SEQ ID NO: 104 - DNA, 40 bp, synthetic construct
  source 1..40: mol_type = other DNA; note = AKM-M7_2R
tcgggaagctgaagnnnntggggcacttagattttacagc

SEQ ID NO: 105 - DNA, 38 bp, synthetic construct
  source 1..38: mol_type = other DNA; note = MSJ_M45_2F
tccgacggtagtgtnnnntcatgctactgcactccagc

SEQ ID NO: 106 - DNA, 37 bp, synthetic construct
  source 1..37: mol_type = other DNA; note = MSJ_M45_2R
tcgggaagctgaagnnnnggaacagacgggccaaact

SEQ ID NO: 107 - DNA, 41 bp, synthetic construct
  source 1..41: mol_type = other DNA; note = AKM-M5_2F
tccgacggtagtgtnnnntgctttctgattggttcccaaaa

SEQ ID NO: 108 - DNA, 43 bp, synthetic construct
  source 1..43: mol_type = other DNA; note = AKM-M5_2R
tcgggaagctgaagnnnnatgacaggaatcctcatatatcttt

SEQ ID NO: 109 - DNA, 42 bp, synthetic construct
  source 1..42: mol_type = other DNA; note = AKM-M2_1F
tccgacggtagtgtnnnnaactcctctaaggaatatgctctc

SEQ ID NO: 110 - DNA, 41 bp, synthetic construct
  source 1..41: mol_type = other DNA; note = AKM-M2_1R
tcgggaagctgaagnnnnacacactagttgcatacatgctt

SEQ ID NO: 111 - DNA, 41 bp, synthetic construct
  source 1..41: mol_type = other DNA; note = AKM13_1F
tccgacggtagtgtnnnnaataaaaaccaccacacagaagg

SEQ ID NO: 112 - DNA, 42 bp, synthetic construct
  source 1..42: mol_type = other DNA; note = AKM13_1R
tcgggaagctgaagnnnngagcttgattaaagtcacaacatc

SEQ ID NO: 113 - DNA, 38 bp, synthetic construct
  source 1..38: mol_type = other DNA; note = LM-M8_1F
tccgacggtagtgtnnnntgtgcttgagtttgttgagc

SEQ ID NO: 114 - DNA, 38 bp, synthetic construct
  source 1..38: mol_type = other DNA; note = LM-M8_1R
tcgggaagctgaagnnnngctcccaaaggaggagaaga

SEQ ID NO: 115 - DNA, 43 bp, synthetic construct
  source 1..43: mol_type = other DNA; note = MSJ-M39_1F
tccgacggtagtgtnnnnacttagctagataaaacacacattt

SEQ ID NO: 116 - DNA, 42 bp, synthetic construct
  source 1..42: mol_type = other DNA; note = MSJ-M39_1R
tcgggaagctgaagnnnnccatgtatcgtatttactggtggt

SEQ ID NO: 117 - DNA, 42 bp, synthetic construct
  source 1..42: mol_type = other DNA; note = LM-M3_1F
tccgacggtagtgtnnnntaacacctcagtggtaactatttt

SEQ ID NO: 118 - DNA, 43 bp, synthetic construct
  source 1..43: mol_type = other DNA; note = LM-M3_1R
tcgggaagctgaagnnnnctttttcagttagtatcactagggc

SEQ ID NO: 119 - DNA, 42 bp, synthetic construct
  source 1..42: mol_type = other DNA; note = AKM-M3_1F
tccgacggtagtgtnnnnggcattcaagtctaacagagctta

SEQ ID NO: 120 - DNA, 39 bp, synthetic construct
  source 1..39: mol_type = other DNA; note = AKM-M3_1R
tcgggaagctgaagnnnntgatgcccatctttcgtactt

SEQ ID NO: 121 - DNA, 41 bp, synthetic construct
  source 1..41: mol_type = other DNA; note = MSJ-M46-1F
tccgacggtagtgtnnnntgaagctccatttatctttggac

SEQ ID NO: 122 - DNA, 44 bp, synthetic construct
  source 1..44: mol_type = other DNA; note = MSJ-M46-1R
tcgggaagctgaagnnnntgtcttacataaatctaaaaccattg

SEQ ID NO: 123 - DNA, 40 bp, synthetic construct
  source 1..40: mol_type = other DNA; note = AKM-M12-F1
tccgacggtagtgtnnnnccactactgtttatgcagcaag

SEQ ID NO: 124 - DNA, 41 bp, synthetic construct
  source 1..41: mol_type = other DNA; note = AKM-M12-R1
tcgggaagctgaagnnnngctggagttttcaaattacactg

SEQ ID NO: 125 - DNA, 41 bp, synthetic construct
  source 1..41: mol_type = other DNA; note = AKM-M16_1F
tccgacggtagtgtnnnnatgcatttgctccttcacacttg

SEQ ID NO: 126 - DNA, 41 bp, synthetic construct
  source 1..41: mol_type = other DNA; note = AKM-M16_1R
tcgggaagctgaagnnnncactgaggcagtatctctgtcat

SEQ ID NO: 127 - DNA, 42 bp, synthetic construct
  source 1..42: mol_type = other DNA; note = AKM_M4_1F
tccgacggtagtgtnnnntggacactgaccaaattgtcaaac

SEQ ID NO: 128 - DNA, 42 bp, synthetic construct
  source 1..42: mol_type = other DNA; note = AKM_M4_1R
tcgggaagctgaagnnnnaagcaattaaacacacttttggtg

SEQ ID NO: 129 - DNA, 41 bp, synthetic construct
  source 1..41: mol_type = other DNA; note = MSJ-M10-F1
tccgacggtagtgtnnnnttcctttccacataaacaacctt

SEQ ID NO: 130 - DNA, 42 bp, synthetic construct
  source 1..42: mol_type = other DNA; note = MSJ-M10-R1
tcgggaagctgaagnnnnctccccaaattgatgtacaggttt

SEQ ID NO: 131 - DNA, 40 bp, synthetic construct
  source 1..40: mol_type = other DNA; note = EJ-M5-F1
tccgacggtagtgtnnnngaggtttcactgctgctttagt

SEQ ID NO: 132 - DNA, 40 bp, synthetic construct
  source 1..40: mol_type = other DNA; note = EJ-M5-R1
tcgggaagctgaagnnnnatgtgactcaagctaccatcct

SEQ ID NO: 133 - DNA, 43 bp, synthetic construct
  source 1..43: mol_type = other DNA; note = LM-M12_1F
tccgacggtagtgtnnnngtgtcactgtggataaagactaaga

SEQ ID NO: 134 - DNA, 40 bp, synthetic construct
  source 1..40: mol_type = other DNA; note = LM-M12_1R
tcgggaagctgaagnnnnagatgcactttgaaacagcctt

SEQ ID NO: 135 - DNA, 42 bp, synthetic construct
  source 1..42: mol_type = other DNA; note = EJ-M1-F3
tccgacggtagtgtnnnntatgtttctgtgcctatattctgc

SEQ ID NO: 136 - DNA, 44 bp, synthetic construct
  source 1..44: mol_type = other DNA; note = EJ-M1-R3
tcgggaagctgaagnnnntgtcatttttgtcttatttcctcaaa

SEQ ID NO: 137 - DNA, 41 bp, synthetic construct
  source 1..41: mol_type = other DNA; note = EJ-M6_2F
tccgacggtagtgtnnnnttgccctttcattagtgtggaaa

SEQ ID NO: 138 - DNA, 36 bp, synthetic construct
  source 1..36: mol_type = other DNA; note = EJ-M6_2R
tcgggaagctgaagnnnngcctcaagggttaggctg

SEQ ID NO: 139 - DNA, 40 bp, synthetic construct
  source 1..40: mol_type = other DNA; note = EJ-M2_1F
tccgacggtagtgtnnnnaccaagagacagaattccacat

SEQ ID NO: 140 - DNA, 38 bp, synthetic construct
  source 1..38: mol_type = other DNA; note = EJ-M2_1R
tcgggaagctgaagnnnngcacaggtgtctttggtgat

SEQ ID NO: 141 - DNA, 43 bp, synthetic construct
  source 1..43: mol_type = other DNA; note = LM-M4-1F
tccgacggtagtgtnnnnatgccatgttccaatcaattctaaa

SEQ ID NO: 142 - DNA, 40 bp, synthetic construct
  source 1..40: mol_type = other DNA; note = LM-M4-1R
tcgggaagctgaagnnnngtggaatgaagaagcgatgtca

SEQ ID NO: 143 - DNA, 39 bp, synthetic construct
  source 1..39: mol_type = other DNA; note = LM-M7-1F
tccgacggtagtgtnnnnatcttggacttccaaaccgct

SEQ ID NO: 144 - DNA, 43 bp, synthetic construct
  source 1..43: mol_type = other DNA; note = LM-M7-1R
tcgggaagctgaagnnnnagttgagactgggtacttaagaaag

SEQ ID NO: 145 - DNA, 39 bp, synthetic construct
  source 1..39: mol_type = other DNA; note = EJ_M4_1F
tccgacggtagtgtnnnnggtgagctctaaccaaatcca

SEQ ID NO: 146 - DNA, 42 bp, synthetic construct
  source 1..42: mol_type = other DNA; note = EJ_M4_1R
tcgggaagctgaagnnnntgggtaattttatttgtgaatggt

SEQ ID NO: 147 - DNA, 43 bp, synthetic construct
  source 1..43: mol_type = other DNA; note = AKM-M6_1F
tccgacggtagtgtnnnnacatggtctttgaagattatccaat

SEQ ID NO: 148 - DNA, 42 bp, synthetic construct
  source 1..42: mol_type = other DNA; note = AKM-M6_1R
tcgggaagctgaagnnnngcaaccaagtagttgaagttaggt

SEQ ID NO: 149 - DNA, 39 bp, synthetic construct
  source 1..39: mol_type = other DNA; note = AKM-M22_1F
tccgacggtagtgtnnnnctttgccccagggtaaaattg

SEQ ID NO: 150 - DNA, 42 bp, synthetic construct
  source 1..42: mol_type = other DNA; note = AKM-M22_1R
tcgggaagctgaagnnnngtcatctcatcacatgaataggaa

SEQ ID NO: 151 - DNA, 76 bp, synthetic construct
  source 1..76: mol_type = other DNA; note = AKMmono07_SR_SIxxxF
caagcagaagacggcatacgagatnnnnnnnnacacgcacgatccgacggtagtgtaaggccttttccctaaggtg

SEQ ID NO: 152 - DNA, 80 bp, synthetic construct
  source 1..80: mol_type = other DNA; note = AKMmono07_SR_UR
aatgatacggcgaccaccgagatctacacatacgagatccgtaatcgggaagctgaagtggggcacttagattttacagc

SEQ ID NO: 153 - DNA, 78 bp, synthetic construct
  source 1..78: mol_type = other DNA; note = AKMmono12_SR_SIxxxF
caagcagaagacggcatacgagatnnnnnnnnacacgcacgatccgacggtagtgtccactactgtttatgcagcaag

SEQ ID NO: 154 - DNA, 75 bp, synthetic construct
  source 1..75: mol_type = other DNA; note = MSJmono20_SR_SIxxxF
caagcagaagacggcatacgagatnnnnnnnnacacgcacgatccgacggtagtgtattgtactccagcacgggt

SEQ ID NO: 155 - DNA, 14 bp, synthetic construct
  source 1..14: mol_type = other DNA
tccgacggtagtgt

SEQ ID NO: 156 - DNA, 14 bp, synthetic construct
  source 1..14: mol_type = other DNA
tcgggaagctgaag

(19) United States
(12) Patent Application Publication
(10) Pub. No.: US 2025/0305054 A1
(43) Pub. Date: Oct. 2, 2025

(54) MICROSATELLITE MARKERS

(71) Applicant: CANCER RESEARCH TECHNOLOGY LIMITED, London (GB)

(72) Inventors: John BURN, Newcastle upon Tyne (GB); Michael Stewart JACKSON, Newcastle upon Tyne (GB); Francisco Mauro SANTIBANEZ-KOREF, Newcastle upon Tyne (GB); Richard GALLON, Newcastle upon Tyne (GB)

(21) Appl. No.: 18/696,028

(22) PCT Filed: Oct. 3, 2022
(86) PCT No.: PCT/GB2022/052500

§ 371 Date: Mar. 27, 2024

(30) Foreign Application Priority Data
Oct. 1, 2021 (GB) 2114136.1

(51) Int. Cl. C12Q 1/6886; C12Q 1/6869

(52) U.S. Cl. CPC C12Q 1/6886; C12Q 1/6869; C12Q 2600/156

(57) ABSTRACT

The invention provides novel methods for evaluating levels of microsatellite instability in a sample and evaluating the biological significance of sequence variations identified in a sample during sequencing. The invention further relates to the use of novel microsatellite instability markers for evaluating levels of microsatellite instability in a sample and evaluating the biological significance of sequence variations identified in a sample during sequencing. Corresponding kits are also provided.

### FIELD OF THE INVENTION

The invention provides novel methods for evaluating levels of microsatellite instability in a sample and evaluating the biological significance of sequence variations identified in a sample during sequencing. The invention further relates to the use of novel microsatellite instability markers for evaluating levels of microsatellite instability in a sample and evaluating the biological significance of sequence variations identified in a sample during sequencing. Corresponding kits are also provided.

### SEQUENCE LISTING

The instant application contains a Sequence Listing which has been submitted electronically in XML format and is hereby incorporated by reference in its entirety. Said XML file, created on Nov. 18, 2022, is named P320245WO_ST26_Sequence_listing_12655892.xml and is 271 kilobytes in size.

### BACKGROUND

The DNA mismatch repair (MMR) system maintains the sequence of the human genome by correcting errors made during DNA replication prior to cell division. MMR deficiency can occur in cancers and results in an increased mutation rate, a high tumor mutation burden, and distinct mutational signatures. Microsatellite instability (MSI), i.e. the increased frequency of insertion and deletion mutations (indels) in short tandem repeats found throughout the human genome, is a well-known and long-used hallmark feature of the mutator phenotype associated with MMR deficiency.

Whilst typically observed in neoplastic cells, MMR deficiency has also been described as a very rare constitutional condition associated with childhood cancer predisposition. This Constitutional MMR deficiency (CMMRD) is caused by germline bi-allelic pathogenic variants affecting one of four MMR genes, and results in a high risk of developing a broad spectrum of malignant tumors within the first three decades of life. Non-malignant clinical features, of which skin pigmentation alterations are the most prevalent, are found in nearly all CMMRD patients and are important diagnostic markers.

Timely diagnosis of CMMRD is critical as it allows patients to benefit from personalized treatment, cancer surveillance, and cancer prevention. Families of CMMRD patients may benefit from identification of affected relatives, and provision of genetic counselling. Due to these important implications, a clinical diagnosis of suspected CMMRD needs confirmation by a molecular diagnosis. However, a definitive genetic diagnosis may be precluded by limitations inherent to any mutational analysis method, specific limitations due to pseudogenes of the PMS2 MMR gene, and variants of uncertain significance (VUS). Hence, complementary functional assays are needed to confirm or refute the diagnosis when genetic analysis fails to render a definite diagnosis.

MSI analysis has been used to detect MMR deficiency in cancers since the discovery of this tumour phenotype in the early 1990s. This test informs the prognosis of the cancer patient, can be used to screen for Lynch syndrome, and may inform use of immunotherapy, such as the immune checkpoint blockade inhibitor pembrolizumab. A wide variety of highly sensitive and specific MSI assays have been developed for tumour diagnostics. Widespread assays include fragment length analysis and software to determine MSI status from high throughput sequencing reads. An example of a commercial kit based on fragment length analysis is the Promega MSI Analysis System, which uses PCR to amplify 5 mononucleotide repeat microsatellite markers, followed by analysis of fluorescently tagged amplicons using capillary electrophoresis to identify microsatellite indels. MSI status is determined by the proportion of microsatellite markers that contain indels. Sequencing-based MSI analysis software use a variety of classification methods and a variety of microsatellites captured by targeted though to whole genome sequencing.

In 2019, the inventors were the first to show that sequencing-based MSI analysis, using single molecule molecular inversion probe (smMIP) amplification of 24 mononucleotide repeats and amplicon sequencing, was able to detect MSI in the non-neoplastic tissues of CMMRD patients (Gallon et al. Hum Mutat. 2019 May; 40(5):649-655, DOI: 10.1002/humu.23721, PMID: 30740824). Prior to this, the weak MSI signal in non-neoplastic CMMRD tissues was only detectable by laborious techniques such as small pool PCR and culturing of lymphoblastoid cell lines, or by fragment length analysis of dinucleotide repeat markers, which are insensitive to MSH6 deficiency and, therefore, ˜25% of CMMRD cases. Other MSI analysis methods used routinely for tumours could not detect this signal.

The inventors' smMIP and sequencing-based MSI assay was initially developed for cancer diagnostics, and hence its 24 mononucleotide repeat markers (herein referred to as the “original markers” which are described in WO2021019197) had been selected from MMR deficient tumour data. Whilst the assay was 98% sensitive and 100% specific for CMMRD detection, there was poor separation of some CMMRD samples from controls (Gallon et al. 2019). A more recent sequencing-based MSI assay has been developed that has a much greater separation of CMMRD from control samples (Gonzalez-Acosta et al. J Med Genet. 2020 April; 57(4):269-273, DOI: 10.1136/jmedgenet-2019-106272; PMID: 31494577). It also uses microsatellite markers selected from MMR deficient tumour data, and improves detection of CMMRD by using exceptionally high read depths per marker (20,000×), and a very large number of microsatellite markers (186 mononucleotide repeats). This second MSI assay for CMMRD detection is therefore limited by a high cost and reliance on high capacity sequencing platforms.

Accordingly, there remains a need for further improved methods for identifying microsatellite instability in a sample.

### SUMMARY OF THE INVENTION

The present invention is based on the inventors' development of a novel panel of MSI markers (listed in Table A below). These markers have been tested and validated in CMMRD samples and surprisingly were found to differentiate between CMMRD and control samples with 100% sensitivity and 100% specificity as shown in the Examples section of the present application. The present inventors have also found that this novel panel of markers is very useful in the context of evaluating MSI in tumours, and therefore can be used to differentiate microsatellite stable (MSS) and MSI cancers. As shown in the Examples section of the present application, the inventors have found that MSI classification of colorectal cancers (CRCs) using the top 24 markers of the new microsatellite marker panel had 100% sensitivity and 100% specificity and provided a very clear separation between microsatellite instability-high (MSI-H) and MSS samples.

The present inventors have found that even just one marker from the novel panel of markers described herein may be sufficient to identify microsatellite instability in a sample. This is because the markers described herein individually have a very high sensitivity and specificity as shown by the markers high AUC ROC scores. Most markers described herein have an AUC ROC score greater than 0.9 (for example 0.91, 0.92, 0.93, 0.94, 0.95, 0.96, 0.97, 0.98, 0.99, or even 1). Merely by way of example, FIG. 8A shows that the marker AKMmono10v2, when analysed on its own, allows separation between CMMRD and control samples. However, it will be appreciated that similar separation of the two types of samples may be expected when analysing any of the markers of the present invention.

Thus, the markers disclosed herein can be used in low cost and scalable MSI assays with improved accuracy for detecting microsatellite instability.

Furthermore, the inventors have surprisingly found that the markers described herein can identify microsatellite instability in a blood sample or part thereof (such as peripheral blood leukocytes). Microsatellite markers that are particularly useful in this context are provided in Table H of the present disclosure.

Moreover, the inventors have developed a set of microsatellite markers which may be particularly useful in a diagnostic context, as the set is optimised for use in a single-round multiplex PCR reaction. The inventors also developed primers that may be used in such a single-round multiplex PCR reaction. These markers and primers are provided in Table I.

Accordingly, in one aspect the present invention provides a method for evaluating levels of microsatellite instability in a sample, the method comprising the steps of:
a) analyzing the sample's DNA to determine the nucleotide sequence of one or more microsatellite marker, wherein the one or more microsatellite marker is selected from Table A;
b) comparing the nucleotide sequence to a predetermined sequence, and determining any deviation, indicative of instability, from the predetermined sequence.

Suitably, the one or more microsatellite markers may be 1, 2, 3, 4, 5, 6, 7, 8, 9, 10, 11, 12, 13, 14, 15, 16, 17, 18, 19, 20, 21, 22, 23, 24, or more, microsatellite markers selected from Table A.

Suitably, at least one of the microsatellite markers may be selected from Table B or Table D.

Suitably, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or more microsatellite markers may be selected from Table B or Table D.

Suitably, at least one of the markers may be selected from the top 21 markers listed in Table B. Suitably, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or 21 of the markers are selected from the top 21 markers listed in Table B.

Suitably, the one or more microsatellite markers selected from Table A may be selected from Table C, optionally wherein at least one of the microsatellite markers may be selected from Table D, further optionally wherein 2, 3, 4, 5, 6, 7, 8, 9, 10, 11, 12, 13, 14, 15, 16, 17, 18, 19, 20, 21, 22, 23 or 24 microsatellite markers may be selected from Table D.

Suitably, at least one of the markers may be selected from the group consisting of AKMmono10v2, LMmono05v2, AKMmono05, and EJmono12_SNP1.

Suitably, the method may comprise the step of amplifying from the sample one or more microsatellite marker selected from Table A to generate microsatellite markers amplicons prior to step a).

In one aspect the present invention provides a method for evaluating the biological significance of sequence variation identified during sequencing, comprising:
a) amplifying from the sample one or more microsatellite marker selected from Table E to generate microsatellite markers amplicons, wherein each microsatellite loci has a single nucleotide polymorphism (SNP) within a short distance of the microsatellite marker and said amplifying step amplifies both the microsatellite marker and associated SNP in a single amplicon;
b) sequencing the amplicons; and
c) comparing the sequences from the amplicons to predetermined sequences and determining any deviation, indicative of instability, from the predetermined sequences; and
d) for heterozygous SNPs, determining whether there is a bias between indel frequencies for the two alleles.

Suitably, the one or more markers may be 1, 2, 3, 4, 5, 6, 7, 8, 9, 10, 11, 12, 13, or 14 markers selected from Table E.

Suitably, at least one of the one or more markers selected from Table E may be AKMmono10v2 or LMmono05v2.

Suitably, the sample may be a fluid sample or a solid sample.

Suitably, the subject may have, be at risk of having, or be predisposed to a condition associated with microsatellite instability.

Suitably, the condition associated with microsatellite instability may be selected from cancer, CMMRD, Lynch syndrome, and Muir-Torre syndrome; preferably cancer or CMMRD.

Suitably, the cancer may be selected from the group consisting of colon cancer, endometrium cancer, gastric cancer, ovarian cancer, hepatobiliary tract cancer, urinary tract cancer, stomach cancer, small intestine cancer, brain cancer, skin cancer, and haematological cancer.

In one aspect, the present invention provides a kit for amplifying one or more microsatellite marker listed in Table A, wherein the kit comprises primers and/or probes for specifically amplifying the one or more microsatellite marker.

Suitably, the microsatellite marker may be associated with a SNP (i.e. is a marker selected from Table E) and wherein the primers and/or probes are for specifically amplifying the one or more microsatellite marker and associated SNP.

In one aspect, the present invention provides use of one or more microsatellite markers selected from Table A for evaluating levels of microsatellite instability in a sample.

In one aspect, the present invention provides use of one or more microsatellite markers selected from Table E for evaluating the biological significance of sequence variation identified during sequencing of a sample.

Throughout the description and claims of this specification, the words “comprise” and “contain” and variations of them mean “including but not limited to”, and they are not intended to (and do not) exclude other moieties, additives, components, integers or steps.

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 2nd ed., Cold Spring Harbor Press, Plainsview, N.Y. (1989); and Ausubel et al., Current Protocols in Molecular Biology (Supplement 47), John Wiley & Sons, New York (1999), for definitions and terms of the art. As a further example, Singleton and Sainsbury, Dictionary of Microbiology and Molecular Biology, 2d Ed., John Wiley and Sons, NY (1994); and Hale and Marham. The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein.

Throughout the description and claims of this specification, the singular encompasses the plural unless the context otherwise requires. In particular, where the indefinite article is used, the specification is to be understood as contemplating plurality as well as singularity, unless the context requires otherwise. Accordingly, as used herein, the singular terms “a”, “an,” and “the” include the plural reference unless the context clearly indicates otherwise.

Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Features, integers, characteristics, compounds, chemical moieties or groups described in conjunction with a particular aspect, embodiment or example of the invention are to be understood to be applicable to any other aspect, embodiment or example described herein unless incompatible therewith.

The entire disclosures of the issued patents, published patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### BRIEF DESCRIPTION OF THE DRAWINGS

In order to provide a better understanding of the present invention, embodiments will be described by way of example only with reference to the following figures, in which:

FIG. 1 shows the ROC AUC for each mononucleotide repeat marker using Reference Allele Frequency (RAF) to classify MMR deficiency in CRC samples. The results are based on samples from the pilot cohort, which contained 8 CMMRD peripheral blood leukocyte genomic DNA samples, 38 control peripheral blood leukocyte genomic DNA samples, 8 MMR deficient CRC genomic DNA samples and 8 MMR proficient CRC genomic DNA.

FIG. 2 show the difference in the median RAF of MMR proficient and MMR deficient CRC samples for each mononucleotide repeat marker. The results are based on samples from the pilot cohort, which contained 8 CMMRD peripheral blood leukocyte genomic DNA samples, 38 control peripheral blood leukocyte genomic DNA samples, 8 MMR deficient CRC genomic DNA samples and 8 MMR proficient CRC genomic DNA.

FIG. 3 shows the ROC AUC for each mononucleotide repeat marker using RAF to classify CMMRD versus control samples. The results are based on samples from the pilot cohort, which contained 8 CMMRD peripheral blood leukocyte genomic DNA samples, 38 control peripheral blood leukocyte genomic DNA samples, 8 MMR deficient CRC genomic DNA samples and 8 MMR proficient CRC genomic DNA.

FIG. 4 shows the difference in the minimum control RAF and the maximum CMMRD RAF for each mononucleotide repeat marker. A more negative difference represents increasing overlap between CMMRD and control RAFs. A more positive difference represents increasing separation between CMMRD and control RAFs. The results are based on samples from the pilot cohort, which contained 8 CMMRD peripheral blood leukocyte genomic DNA samples, 38 control peripheral blood leukocyte genomic DNA samples, 8 MMR deficient CRC genomic DNA samples and 8 MMR proficient CRC genomic DNA.

FIG. 5 shows the MSI assay score of the blinded cohort and known controls using 32 of the new mononucleotide repeat markers, and the scoring method described by Gallon et al. 2019 and Perez-Valencia et al. 2020. The results are based on samples from the large blinded cohort, which contained 30 CMMRD peripheral blood leukocyte genomic DNA samples, 73 control peripheral blood leukocyte genomic DNA samples (43 blinded and 30 known controls).

FIG. 6 shows a comparison of MSI assay score from the blinded cohort and known controls using either the original 24 mononucleotide repeat markers or new 32 mononucleotide repeat markers, and the scoring method described by Gallon et al. 2019 and Perez-Valencia et al. 2020. The dotted lines represent the minimum CMMRD score, and the solid lines represent the maximum control or LS score. The results are based on samples from the large blinded cohort, which contained 30 CMMRD peripheral blood leukocyte genomic DNA samples, 73 control peripheral blood leukocyte genomic DNA samples (43 blinded and 30 known controls).

FIG. 7 shows a comparison of microsatellite marker length (in nucleotides) and ROC AUC (using single molecule sequence [smSequence)] RAF as a measure of MSI) to detect CMMRD in the blinded cohort and known controls. The results are based on samples from the large blinded cohort, which contained 30 CMMRD peripheral blood leukocyte genomic DNA samples, 73 control peripheral blood leukocyte genomic DNA samples (43 blinded and 30 known controls).

FIG. 8(A) shows a summary of MSI assay scores from the blinded cohort and known controls using different numbers of markers from ranking both new and original marker sets. It can be seen that use of a single marker provided separating between all CMMRD and control samples. (B) shows the same results as FIG. 8A, but the y axis has been limited to show the separation of CMMRD and control scores at low marker numbers from ranking both new and original marker sets. (C) shows summary of MSI assay scores from the blinded cohort and known controls using different numbers of markers from ranking the original marker set only. A persistent overlap between CMMRD and control samples can be seen. (D) shows the same data as FIG. 8C, but the y axis has been limited to show the overlap of CMMRD and control scores with any marker combination from ranking the original marker set only. The results are based on samples from the large blinded cohort, which contained 30 CMMRD peripheral blood leukocyte genomic DNA samples, 73 control peripheral blood leukocyte genomic DNA samples (43 blinded and 30 known controls).

FIG. 9(A) shows the range of MSI assay scores in control and CMMRD samples, as well as the margin difference (minimum CMMRD score−maximum control score) and median difference (median CMMRD score−median control score) in CMMRD and control scores, in the blinded cohort and known controls using different numbers of markers from ranking both new and original marker sets. (B) shows the range of MSI assay scores in control and CMMRD samples, as well as the margin difference (minimum CMMRD score−maximum control score) and median difference (median CMMRD score−median control score) in CMMRD and control scores, in the blinded cohort and known controls using different numbers of markers from ranking the original marker set only. The results are based on samples from the large blinded cohort, which contained 30 CMMRD peripheral blood leukocyte genomic DNA samples, 73 control peripheral blood leukocyte genomic DNA samples (43 blinded and 30 known controls).

FIG. 10(A) shows the normalised margin difference ((minimum CMMRD score−maximum control score)/range control score) and the normalised median difference ((median CMMRD score−median control score)/range control score) in MSI assay score in control and CMMRD samples from the blinded cohort and known controls using different numbers of markers from ranking both new and original marker sets. (B) shows normalised margin difference ((minimum CMMRD score−maximum control score)/range control score) and the normalised median difference ((median CMMRD score−median control score)/range control score) in MSI assay score in control and CMMRD samples from the blinded cohort and known controls using different numbers of markers from ranking the original marker set only. The results are based on samples from the expanded cohort, which contained 30 CMMRD peripheral blood leukocyte genomic DNA samples, 73 control peripheral blood leukocyte genomic DNA samples (43 blinded and 30 known controls).

FIG. 11 shows ROC AUC for each mononucleotide repeat marker from both new and original microsatellite marker sets, calculated from read RAFs of 50 MSI-H and 52 MSS CRCs.

FIG. 12 shows a comparison of MSI assay score from 50 MSI-H and 52 MSS CRCs using either the original 24 mononucleotide repeat markers or top 24 new mononucleotide repeat markers, and the classification method described by Redford et al. 2018 and used by Gallon et al. 2020. The dotted lines represent the minimum MSI-H CRC score, and the solid lines represent the maximum MSS CRC score.

FIG. 13 shows a comparison of microsatellite marker length (in nucleotides) and ROC AUC, calculated from read RAF of 50 MSI-H and 52 MSS CRCs, of both new and original microsatellite marker sets.

FIG. 14 shows summary of MSI assay scores from 50 MSI-H and 52 MSS CRCs from ranking the new microsatellite marker set and classification using different numbers of the top ranked markers (A) and using the original microsatellite marker set (B).

FIG. 15 shows summary of margin difference (minimum MSI-H CRC score−maximum MSS CRC score), median difference (median MSI-H CRC score−median MSS CRC score), and range in MSI assay scores from 50 MSI-H and 52 MSS CRCs from ranking the new microsatellite marker set and classification using different numbers of the top ranked markers (A) and using the original microsatellite marker set (B).

FIG. 16 shows the normalised margin difference ((minimum MSI-H CRC score−maximum MSS CRC score)/range MSS CRC scores) and the normalised median difference ((median MSI-H CRC score−median MSS CRC score)/range MSS CRC scores) in MSI assay score of 50 MSI-H and 52 MSS CRCs from ranking the new microsatellite marker set and classification using different numbers of the top ranked markers (A) and using the original microsatellite marker set (B).

FIG. 17 Whole genome sequencing and pilot amplicon sequencing to select MSI markers. The frequency of variant microsatellites by motif size in whole genome sequence data from blood, including the raw count of microsatellites containing a variant for each sample (A), and the relative frequency of non-germline microsatellite variants for each sample (B). Candidate MSI marker performance in amplicon sequence data from a pilot cohort of peripheral blood leukocyte (PBL) and colorectal cancers (CRC) samples, quantified by the receiver operator characteristic area under curve (ROC AUC) of microsatellite reference allele frequency (RAF) to discriminate between MMR-deficient and -proficient samples (C), and by the difference in median RAF between MMR-deficient and MMR-proficient samples (D).

FIG. 18 shows sample MSI scores. The MSI scores of a blinded cohort of 56 CMMRD, 8 CMMRD-negative, and 43 control peripheral blood leukocyte (PBL) gDNAs, 80 reference control PBL gDNAs, and 40 Lynch syndrome PBL gDNAs using 32 new MSI markers (A). CMMRD-negative refers to patients with a CMMRD-like phenotype but no MMR variants at germline analysis. A comparison of initial and repeat MSI scores of 26 CMMRD and 33 control PBL gDNAs (B).

FIG. 19 MSI scores of blood samples by sequencing batch. Data for repeat amplification and sequencing of samples are shown.

FIG. 20 Receiver operator characteristic area under curve (ROC AUC) values calculated from the ability of each MSI marker to separate CMMRD blood from control samples using microsatellite reference allele frequency (RAF), comparing new and original marker sets.

FIG. 21 MSI marker characteristics and performance. A comparison of the length of each MSI marker and its receiver operator characteristic area under curve (ROC AUC) to discriminate between CMMRD and control PBL samples (A). A comparison of MSI score of 50 CMMRD and 75 control PBL samples using either the original 24 tumour-derived MSI markers or an equivalent number of the most discriminatory of the new blood-derived MSI markers (B).

FIG. 22 MSI scores of blood samples using reduced panels of the most discriminatory N of the original MSI markers (left panel) and most discriminatory N of the new MSI markers (right panel).

FIG. 23 shows MSI scores. As a further test of diagnostic utility, a larger panel of 54 new MSI markers was smMIP-amplified and sequenced in 192 colorectal cancers (CRCs) of known MSI status (MSI Analysis System v1.2, Promega) as a biomarker of MMR function. A larger panel of MSI markers could be used as, previously, the inventors have shown that smSequences provide no benefit to CRC MSI classification. Therefore, lower read depths of 3000× can be used, and hence more MSI markers assessed for equivalent cost. Custom R scripts were used to extract microsatellite variants from reads. The microsatellite deletion frequencies and allelic bias (if a heterozygous neighbouring SNP was available to discriminate between paternal and maternal alleles) in sequence reads generated from a training cohort of 50 MSI-H and 52 MSS CRCs were used to train a naïve Bayesian classifier according to Redford et al. The remaining 90 CRCs (46 MSI-H, 44 MSS) formed the validation cohort. A tumour-MSI score was generated for each sample using the trained classifier. Tumour-MSI scores >0 indicate a higher probability the sample is MMR deficient than MMR proficient, and the inverse for scores <0.

Tumour-MSI scoring achieved 100% sensitivity (50/50; 95% CI: 92.9-100.0%) and 100% specificity (52/52; 95% CI: 93.2-100.0%) in the training cohort and 100% sensitivity (46/46; 95% CI: 92.3-100.0%) and 100% specificity (44/44; 95% CI: 92.0-100.0%) in the validation cohort (A). Training cohort samples were also analysed by the original MSI markers. Each marker's ability to separate MMR deficient and MMR proficient CRCs by microsatellite reference allele frequency (RAF) in the training cohort data was assessed. RAF ROC AUCs of the new MSI markers were greater than the RAF ROC AUCs of the originals (p=8.31×10⁻⁵) (B). To compare tumour-MSI classification by marker set with an equivalent number of MSI markers, the new MSI markers were ranked by ROC AUC and the most discriminatory 24 were used to re-score the training cohort samples, achieving 100% accuracy as for the full 54 marker panel (C). Scoring of the training cohort by the original MSI markers misclassified two CRCs, one MMR deficient (49/50; 98% sensitivity, 95% CI: 89.4-99.9%) and one MMR proficient (51/52; 98% sensitivity, 95% CI: 89.7-99.9%) (C). MMR deficient CRCs had more positive tumour-MSI scores when using new versus original MSI markers (p=3.16×10⁻⁴) and MMR proficient CRCs had more negative scores when using new versus original MSI markers (p=2.23×10⁻¹⁴), demonstrating a greater score-separation with the new MSI markers. The most discriminatory 24 new MSI markers also classified the validation cohort with 100% accuracy as for the full 54 marker panel (D).

FIG. 24 shows sample MSI scores by patient genotype. The MSI scores of CMMRD patients by whether they have at least one MMR missense variant in their germline (A). A pair-wise comparison of the MSI scores of CMMRD patients who share the same MMR genotype (B).

FIG. 25 shows associations of disease phenotype with MSI score or MMR genotype. The MSI score and age of first tumour of 50 CMMRD patients (A). The age of first tumour of 50 CMMRD patients by whether they have at least one MMR missense variant in their germline (B).

FIG. 26 shows sample MSI scores compared to patient age and presence of tumour at sample collection. The MSI score and age of sample collection of 30 CMMRD patients (A). The MSI score by whether the patient had a tumour at sample collection for 27 CMMRD patients (B).

FIG. 27 MSI scores of a training and validation cohort of FFPE CRCs, NEQAS standards, and cancer cell lines (A). The microsatellite allele length and allele frequency distribution of the 24 original and 32 new MSI markers in 75 control blood samples, 50 CMMRD blood samples, 52 microsatellite stable (MSS) colorectal cancers (CRCs), and 50 MSI-high (MSI-H) CRCs for which sequence data from both marker sets were available (B).

### DETAILED DESCRIPTION

The present invention is based on the inventors' identification of new, highly accurate markers for evaluating microsatellite instability (MSI). The identification of these new markers allows the design and implementation of new MSI screening methods using a smaller number of microsatellite markers than previously thought possible. For example, prior to the identification of the markers disclosed herein, differentiation between CMMRD and control samples required analyzing 186 MSI markers (Gonzalez-Acosta et al. 2020). Surprisingly, using the markers disclosed herein, this may be achieved by analyzing just one of the microsatellite markers listed in Table A (for example using marker AKMmono10v2, LMmono05v2, AKMmono05 or EJmono12_SNP1). Furthermore, the inventors found that these markers are not only highly accurate in the context of detecting MSI associated with CMMRD, but may also be superior than previously disclosed microsatellite markers differentiating between MSS and MSI cancers. Additionally, the inventors have surprisingly found that these microsatellite markers enable the evaluation of microsatellite instability not only in a solid sample (such as solid tumour sample), but also in a fluid sample (such as a blood sample or urine sample).

Accordingly, in one aspect, provided herein is a method for evaluating levels of microsatellite instability in a sample, comprising:

a) analyzing the sample's DNA to determine the nucleotide sequence of one or more microsatellite marker, wherein the one or more microsatellite marker is selected from Table A;

b) comparing the nucleotide sequence to a predetermined sequence, and determining any deviation, indicative of instability, from the predetermined sequences.

In addition, some of the 62 markers are associated with a single nucleotide polymorphism (SNP) located within a short distance of the marker. Using markers associated with such SNPs can differentiate between amplification and/or sequencing errors, and MSI induced indels/mutations. Such SNPs are typically within 80 base pairs of the associated microsatellite marker, for example 50 base pairs, 40 base pairs, or 30 base pairs. Suitably, the single SNP has a minor allele frequency of above 0.05. Suitably, the SNP has a high heterozygosity. Accordingly, the invention also provides novel methods for evaluating the biological significance of sequence variation identified in a microsatellite marker listed in Table E.

In general, microsatellites are mono-, di-, tri-, tetra-, penta-, or hexanucleotide repeats found in DNA, consisting of at least two units and with a minimal length of 6 bases. Homopolymers are a particular subclass of microsatellites, which are mononucleotide repeats of at least 6 bases; in other words, a stretch of at least 6 consecutive A, C, T or G residues if looking at the DNA level. The microsatellite markers disclosed herein are homopolymers. The terms “microsatellite marker, “microsatellite instability marker”, and “marker” are used herein interchangeably and have the same meaning.

Microsatellite instability (MSI) as used herein refers to a unique molecular alteration and hyper-mutable phenotype, which is the result of a defective DNA mismatch repair (MMR) system, and can be defined as the presence of alternate sized repetitive DNA sequences as compared to a predetermined (for example reference) sequence. Suitably, in the context of the present disclosure, DNA may refer to genomic DNA. Suitably, the DNA may be cell free DNA. Alternate sized repetitive DNA sequence may be due to “an indel”. An “indel” as used herein refers to a mutation class that includes insertions, deletions, or a combination thereof. An indel in a microsatellite region results in a net gain or loss of nucleotides. The presence of an indel can be established by comparing it to DNA in which the indel is not present (e.g. comparing DNA from a tumour sample to germline DNA from the subject with the tumour), or, by comparing it to a reference (predetermined) length of the microsatellite (e.g. Human reference genomes). Comparison may involve counting the number of repeated units. In the context of the present disclosure, a deviation indicative of instability is an alternate sized repetitive DNA sequences, for example due to an indel.

The term “evaluating levels” as used herein refers to determining the presence or absence of microsatellite instability in a subject or sample obtained from the subject. Suitably, when the presence of microsatellite instability has been determined in a sample, the MSI status may be then determined by calculating the percentage of microsatellite markers that were found to have a deviation indicative of instability. MSI status can be one of two discrete classes: MSI-H (also referred to as MSI-high, MSI positive or MSI) or MSI-L (also referred to as MSI-low). Typically, to be classified as MSI-H, at least 30% of the markers used to classify MSI status need to score positive (i.e. have a deviation indicative of instability). If an intermediate number of markers scores positive (that is less than 30% but more than 0%), then the MSI status is classified as MSI-L. An absence of microsatellite instability may also be referred to as microsatellites stability (MSS).

As used herein, the noun “subject” refers to an individual vertebrate, more particularly an individual mammal, most particularly an individual human being. Suitably, the subject may be a human, but can also be a different mammal, particularly a domestic animal such as cat, dog, rabbit, guinea pig, ferret, rat, mouse, and the like, or a farm animal like horse, cows, pig, goat, sheep, llama, and the like. A subject can also be a non-mammalian vertebrate, like a fish, reptile, amphibian or bird; in essence any animal which can develop cancer fulfils the definition. Suitably, the subject has, is suspected of having, is at risk of having or is predisposed to a condition associated with microsatellite instability. Conditions associated with microsatellite instability can include one or more of: cancer conditions (e.g., colon cancer, gastric cancer, endometrium cancer, ovarian cancer, hepatobiliary tract cancer, urinary tract cancer, stomach cancer, small intestine cancer, brain cancer, skin cancer, haematological cancer, or any other solid or liquid malignant neoplasia); CMMRD, Lynch syndrome; Muir-Torre syndrome; and/or any other suitable conditions associated with mismatch repair deficiency. Haematological cancers can acquire MMR deficiency in therapy-resistant clones and therefore MSI analysis may be relevant to relapsed tumours even though MSI/MMR deficiency is rare in primary tumours. Lynch syndrome as used herein refers to an autosomal dominant genetic condition which has a high risk of colon cancer as well as other cancers including endometrium, ovary, stomach, small intestine, hepatobiliary tract, upper urinary tract, brain, and skin cancer. The increased risk for these cancers is due to inherited mutations that impair DNA mismatch repair. The old name for the condition is Hereditary Non-Polyposis Colorectal Cancer (HNPCC).

The term “sample” as used herein refers to samples comprising biological material and, in particular, DNA of the subject (or subject's cancer). Suitably, the sample may be a fluid sample (such as blood, plasma, serum, saliva or urine, or part thereof), or a solid sample (such as a tissue biopsy for example of a tumour). Suitably, the solid sample may be formalin-fixed paraffin-embedded. Techniques for obtaining and preparing the aforementioned types of biological samples are well known in the art. In the context of the present disclosure, a part of a fluid sample includes cells that are present within the fluid sample. By way of example, when the fluid sample is a blood sample, a part of the blood sample may be peripheral blood leukocytes and/or cell free DNA present with the blood sample. Thus, in a suitable embodiment, the sample may be a peripheral blood leukocyte sample. Such a sample may be particularly suitable in a method of the invention where the microsatellite marker is selected from Table H.

Testing biological samples using the methods described herein may be particularly useful e.g. for early cancer detection in those at high risk of cancer (for example diagnosed with CMMRD) or monitoring for disease recurrence (by assessing circulating tumour or cell free DNA). The term “cancer” as used herein, refers a disease involving unregulated cell growth, also referred to as malignant neoplasm. The term “tumour” is used as a synonym in the application. It is envisaged that this term covers all solid tumour types (carcinoma, sarcoma, blastoma), but it also explicitly encompasses non-solid cancer types such as leukemia. Thus, a “tumour sample” encompasses both solid tumour samples (e.g. tissue biopsies) as well as biological fluid samples (e.g. those that have been obtained or isolated from a bodily fluid such as urine, blood, plasma, serum etc). As would be clearly understood by a person of skill in the art, the sample can be described as a “sample of tumour DNA”. The tumour DNA may be present within a bodily fluid such as urine, blood, plasma, serum etc and may be isolated from the bodily fluid prior to performing the methods described herein. Any appropriate method for obtaining or isolating the tumour DNA may be used. Several appropriate methods are well known in the art. Typically, a sample of tumour DNA has at one point been isolated from a subject, particularly a subject with cancer. Optionally, it has undergone one or more forms of pre-treatment (e.g. lysis, fractionation, separation, purification) in order for the DNA to be sequenced, although it is also envisaged that DNA from an untreated sample may be sequenced.

In the context of the present disclosure, the nucleotide sequence may be determined by sequencing (for example genomic DNA sequencing or amplicon sequencing). As used herein, “sequencing” refers to biochemical methods for determining the order of the nucleotide bases, adenine, guanine, cytosine, and thymine, in a DNA oligonucleotide. Methods of sequencing will be well known to those skilled in the art. Merely by way of example, sequencing may be by a method selected from group consisting of high throughput sequencing, next generation sequencing, sequencing-by-synthesis, ion semiconductor sequencing and/or pyrosequencing.

Suitably, prior to determining the nucleotide sequence of the one or more microsatellite marker (for example by sequencing), the microsatellite marker may be amplified. In such embodiments, the methods provided herein may compare the sequences from the microsatellite amplicons to predetermined sequences and determine any deviation, indicative of instability, from the predetermined sequences. Methods for detecting an insertion or deletion are well known in the art.

Accordingly, the method for evaluating levels of microsatellite instability in a sample, may comprise:
a) amplifying from the sample one or more microsatellite marker selected from Table A to generate microsatellite markers amplicons,
b) analyzing the amplicons to determine the nucleotide sequence of one or more microsatellite marker;
c) comparing the nucleotide sequence to a predetermined sequence, and determining any deviation, indicative of instability, from the predetermined sequences.

Although the invention is exemplified herein using molecular inversion probes (MIPs; e.g. single-molecule molecular inversion probes (smMIPs)) to amplify the selected markers, any other appropriate technique for amplifying the selected loci may be used. Alternative appropriate methods are well known in the art and include conventional PCR. In other words, the methods may use any appropriate nucleic acid sequence (e.g. primer and/or probe) that enables amplification of the selected markers. The amplification step may amplify each selected microsatellite marker individually (in a separate reaction), or may comprise co-amplifying some or all of the selected markers in a multiplex amplification reaction. Suitable primers and/or probes may be selected for the chosen method using standard techniques. Suitably, in methods wherein a single nucleotide polymorphism (SNP) within a short distance of the selected microsatellite marker is to be amplified together with the marker in order to generate a single amplicon encompassing the maker and the SNP, primers and/or probes that amplify both the microsatellite marker and the SNP within a short distance of the microsatellite marker need to be used.

The primers and/or probes may contain a sequence of sufficient length and complementarity to a corresponding DNA region to specifically hybridize with that region under suitable hybridization conditions. The corresponding DNA region may be the region of the microsatellite marker itself, or a region up or downstream of the microsatellite marker (or marker and SNP). Sequences of exemplary probes are provided in Table F. These probes give rise to the kits of the present disclosure, which are described in more detail elsewhere in the present specification.

In the context of the present disclosure, multiplex amplification and sequencing techniques may be particularly advantageous because they allow for automated sequence analysis and high throughput diagnostics. However, as would be clear to a person of skill in the art, any other suitable means for amplifying and sequencing the informative MSI markers described herein may also be used (e.g. conventional PCR may be used).

Upon determination of the nucleotide sequence of one or more microsatellite marker, the nucleotide sequence is compared to a predetermined sequence in order to determine any deviation indicative of instability from the predetermined sequences. The deviation may be an indel when compared to the predetermined sequences. The predetermined sequence (also referred to as a reference sequence) may be a sequence of said microsatellite marker in a healthy control, for example a subject or group of subjects believed or known to not have, not be at risk of, or not be predisposed to a microsatellite instability associated condition. However, one of the advantages of the methods described herein is that accurate MSI classification using the methods provided herein does not require control normal DNA and MSI may be determined by simply counting the number of repeats.

The methods of the present invention comprise determining the nucleotide sequence of one or more microsatellite marker, wherein the one or more microsatellite marker is listed in Table A.

Suitably, the one or more microsatellite markers is 1, 2, 3, 4, 5, 6, 7, 8, 9, 10, 11, 12, 13, 14, 15, 16, 17, 18, 19, 20, 21, 22, 23, 24, 28, 32 or more, microsatellite markers listed in Table A.

Suitably at least one of the microsatellite markers is selected from Table B, Table D, Table H, or Table I; or at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or more microsatellite markers are selected from Table B, Table D, Table H or Table I.

More suitably, at least one of the markers is selected from the top 21 markers listed in Table B. Suitably, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or 21 are selected from the top 21 markers listed in Table B. Suitably, the one or more markers selected from the top 21 markers listed in Table B may be in combination with one or more other markers listed in Tables A, B, C, D, H or I.

Suitably, the one or more microsatellite markers listed in Table A is selected from the group of microsatellite markers listed in Table C, optionally at least one of the microsatellite markers is selected from Table D, or at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, or 24 microsatellite markers are selected from Table D.

Suitably, when the method comprises the step of determining the nucleotide sequence of one microsatellite marker, the microsatellite marker may be any one of the markers listed in Table A, B, C, D, H or I.

Suitably, the methods of the present invention comprise determining the nucleotide sequence of one or more microsatellite marker, wherein the one or more microsatellite marker is listed in Table H. Suitably, the one or more microsatellite markers is 1, 2, 3, 4, 5, 6, 7, 8, 9, 10, 11, 12, 13, 14, 15, 16, 17, 18, 19, 20, 21, 22, 23, 24, 28, or more microsatellite markers listed in Table H. More suitably, the one or more microsatellite markers is 24 or more, microsatellite markers listed in Table H. More suitably, the one or more microsatellite markers is 32 markers listed in Table H. In an embodiment where the method comprises determining the nucleotide sequence of one or more microsatellite marker selected from Table H (for example 24 or more, or all 32 markers listed in Table H), the sample may be a fluid sample (such a blood sample, or part thereof, for example peripheral blood leukocytes). Suitably, when the one or more microsatellite marker is listed in Table H, the sample is a blood sample or part thereof, for example PBLs.

Suitably, the methods of the present invention comprise determining the nucleotide sequence of one or more microsatellite marker, wherein the one or more microsatellite marker is listed in Table I. Suitably, the one or more microsatellite markers is 1, 2, 3, 4, 5, 6, 7, 8, 9, 10, 11, 12, 13, or 14 microsatellite markers listed in Table I. More suitably, the one or more microsatellite markers is 10, 11, 12, 13 or 14 microsatellite markers listed in Table I. More suitably, the one or more microsatellite markers is the 14 microsatellite markers listed in Table I.

Suitably, the microsatellite markers disclosed herein may be amplified in a multiplex PCR round reaction. Suitably, the multiplex PCR method may be a single-round or two-round multiplex PCR method, more suitably single-round multiplex PCR method. Suitably, the single-round multiplex PCR may involve amplifying form a sample one or more marker (for example 2, 3, 4, 5, 6, 7, 8, 9, 10, 11, 12, 13, or 14 microsatellite markers) listed in Table I.

Suitably, the markers may be amplified using the primers comprising or consisting of the sequences as shown in Table I, prior to determining the nucleotide sequences of the markers.

Suitably, the method comprises determining the nucleotide sequence of one or more microsatellite marker selected from the group consisting of AKMmono10v2, LMmono05v2, AKMmono05 and EJmono12_SNP1.

Suitably, the method comprises determining the nucleotide sequence of one or more microsatellite marker selected from the group consisting of EJmono12_SNP1, LMmono05v2_SNP1, AKMmono14_SNP1 and MSJmono22_SNP1.

Suitably, the method comprises determining the nucleotide sequence of one or more microsatellite marker selected from the group consisting of EJmono12_SNP1, LMmono05v2_SNP1, AKMmono14_SNP1, MSJmono22_SNP1 and EJmono14v2_SNP1.

Suitably, the method comprises determining the nucleotide sequence of one or more microsatellite marker selected from the group consisting of EJmono12_SNP1, LMmono05v2_SNP1, AKMmono14_SNP1, MSJmono22_SNP1, EJmono14v2_SNP1 and MSJmono20_SNP1.

Suitably, the method comprises determining the nucleotide sequence of one or more microsatellite marker group consisting of EJmono12_SNP1, LMmono05v2_SNP1, AKMmono14_SNP1, MSJmono22_SNP1, EJmono14v2_SNP1, MSJmono20_SNP1 and AKMmono07_SNP1.

Suitably, the method comprises determining the nucleotide sequence of one or more microsatellite marker selected from the group consisting of EJmono12_SNP1, LMmono05v2_SNP1, AKMmono14_SNP1, MSJmono22_SNP1, EJmono14v2_SNP1, MSJmono20_SNP1, AKMmono07_SNP1 and AKMmono05_SNP1.

Suitably, the method comprises determining the nucleotide sequence of one or more microsatellite marker selected from the group consisting of EJmono12_SNP1, LMmono05v2_SNP1, AKMmono14_SNP1, MSJmono22_SNP1, EJmono14v2_SNP1, MSJmono20_SNP1, AKMmono07_SNP1, AKMmono05_SNP1 and LMmono09_SNP1.

Suitably, the method comprises determining the nucleotide sequence of one or more microsatellite marker selected from the group consisting of EJmono12_SNP1, LMmono05v2_SNP1, AKMmono14_SNP1, MSJmono22_SNP1, EJmono14v2_SNP1, MSJmono20_SNP1, AKMmono07_SNP1, AKMmono05_SNP1, LMmono09_SNP1 and AKMmono02_SNP1.

Suitably, the method comprises determining the nucleotide sequence of one or more microsatellite marker selected from the group consisting of EJmono12_SNP1, LMmono05v2_SNP1, AKMmono14_SNP1, MSJmono22_SNP1, EJmono14v2_SNP1, MSJmono20_SNP1, AKMmono07_SNP1, AKMmono05_SNP1, LMmono09_SNP1, AKMmono02_SNP1 and AKMmono13_SNP1.

Suitably, the method comprises determining the nucleotide sequence of one or more microsatellite marker selected from the group consisting of EJmono12_SNP1, LMmono05v2_SNP1, AKMmono14_SNP1, MSJmono22_SNP1, EJmono14v2_SNP1, MSJmono20_SNP1, AKMmono07_SNP1, AKMmono05_SNP1, LMmono09_SNP1, AKMmono02_SNP1, AKMmono13_SNP1 and LMmono08_SNP1.

Suitably, the method comprises determining the nucleotide sequence of one or more microsatellite marker selected from the group consisting of EJmono12_SNP1, LMmono05v2_SNP1, AKMmono14_SNP1, MSJmono22_SNP1, EJmono14v2_SNP1, MSJmono20_SNP1, AKMmono07_SNP1, AKMmono05_SNP1, LMmono09_SNP1, AKMmono02_SNP1, AKMmono13_SNP1, LMmono08_SNP1 and MSJmono39_SNP1.

Suitably, the method comprises determining the nucleotide sequence of one or more microsatellite marker selected from the group consisting of EJmono12_SNP1, LMmono05v2_SNP1, AKMmono14_SNP1, MSJmono22_SNP1, EJmono14v2_SNP1, MSJmono20_SNP1, AKMmono07_SNP1, AKMmono05_SNP1, LMmono09_SNP1, AKMmono02_SNP1, AKMmono13_SNP1, LMmono08_SNP1, MSJmono39_SNP1 and LMmono03_SNP1.

Suitably, the method comprises determining the nucleotide sequence of one or more microsatellite marker selected from the group consisting of EJmono12_SNP1, LMmono05v2_SNP1, AKMmono14_SNP1, MSJmono22_SNP1, EJmono14v2_SNP1, MSJmono20_SNP1, AKMmono07_SNP1, AKMmono05_SNP1, LMmono09_SNP1, AKMmono02_SNP1, AKMmono13_SNP1, LMmono08_SNP1, MSJmono39_SNP1, LMmono03_SNP1 and AKMmono03_SNP1.

Suitably, the method comprises determining the nucleotide sequence of one or more microsatellite marker selected from the group consisting of EJmono12_SNP1, LMmono05v2_SNP1, AKMmono14_SNP1, MSJmono22_SNP1, EJmono14v2_SNP1, MSJmono20_SNP1, AKMmono07_SNP1, AKMmono05_SNP1, LMmono09_SNP1, AKMmono02_SNP1, AKMmono13_SNP1, LMmono08_SNP1, MSJmono39_SNP1, LMmono03_SNP1, AKMmono03_SNP1, and MSJmono27_SNP1.

Suitably, the method comprises determining the nucleotide sequence of one or more microsatellite marker selected from the group consisting of EJmono12_SNP1, LMmono05v2_SNP1, AKMmono14_SNP1, MSJmono22_SNP1, EJmono14v2_SNP1, MSJmono20_SNP1, AKMmono07_SNP1, AKMmono05_SNP1, LMmono09_SNP1, AKMmono02_SNP1, AKMmono13_SNP1, LMmono08_SNP1, MSJmono39_SNP1, LMmono03_SNP1, AKMmono03_SNP1, MSJmono27_SNP1 and MSJmono46_SNP1.

Suitably, the method comprises determining the nucleotide sequence of one or more microsatellite marker selected from the group consisting of EJmono12_SNP1, LMmono05v2_SNP1, AKMmono14_SNP1, MSJmono22_SNP1, EJmono14v2_SNP1, MSJmono20_SNP1, AKMmono07_SNP1, AKMmono05_SNP1, LMmono09_SNP1, AKMmono02_SNP1, AKMmono13_SNP1, LMmono08_SNP1, MSJmono39_SNP1, LMmono03_SNP1, AKMmono03_SNP1, MSJmono27_SNP1, MSJmono46_SNP1 and MSJmono11_SNP1.

Suitably, the method comprises determining the nucleotide sequence of one or more microsatellite marker selected from the group consisting of EJmono12_SNP1, LMmono05v2_SNP1, AKMmono14_SNP1, MSJmono22_SNP1, EJmono14v2_SNP1, MSJmono20_SNP1, AKMmono07_SNP1, AKMmono05_SNP1, LMmono09_SNP1, AKMmono02_SNP1, AKMmono13_SNP1, LMmono08_SNP1, MSJmono39_SNP1, LMmono03_SNP1, AKMmono03_SNP1, MSJmono27_SNP1, MSJmono46_SNP1, MSJmono11_SNP1 and AKMmono12_SNP1.

Suitably, the method comprises determining the nucleotide sequence of one or more microsatellite marker selected from the group consisting of EJmono12_SNP1, LMmono05v2_SNP1, AKMmono14_SNP1, MSJmono22_SNP1, EJmono14v2_SNP1, MSJmono20_SNP1, AKMmono07_SNP1, AKMmono05_SNP1, LMmono09_SNP1, AKMmono02_SNP1, AKMmono13_SNP1, LMmono08_SNP1, MSJmono39_SNP1, LMmono03_SNP1, AKMmono03_SNP1, MSJmono27_SNP1, MSJmono46_SNP1, MSJmono11_SNP1, AKMmono12_SNP1 and MSJmono40_SNP1.

Suitably, the method comprises determining the nucleotide sequence of one or more microsatellite marker selected from the group consisting of EJmono12_SNP1, LMmono05v2_SNP1, AKMmono14_SNP1, MSJmono22_SNP1, EJmono14v2_SNP1, MSJmono20_SNP1, AKMmono07_SNP1, AKMmono05_SNP1, LMmono09_SNP1, AKMmono02_SNP1, AKMmono13_SNP1, LMmono08_SNP1, MSJmono39_SNP1, LMmono03_SNP1, AKMmono03_SNP1, MSJmono27_SNP1, MSJmono46_SNP1, MSJmono11_SNP1, AKMmono12_SNP1, MSJmono40_SNP1 and EJmono03_SNP1.

Suitably, the method comprises determining the nucleotide sequence of one or more microsatellite marker selected from the group consisting of EJmono12_SNP1, LMmono05v2_SNP1, AKMmono14_SNP1, MSJmono22_SNP1, EJmono14v2_SNP1, MSJmono20_SNP1, AKMmono07_SNP1, AKMmono05_SNP1, LMmono09_SNP1, AKMmono02_SNP1, AKMmono13_SNP1, LMmono08_SNP1, MSJmono39_SNP1, LMmono03_SNP1, AKMmono03_SNP1, MSJmono27_SNP1, MSJmono46_SNP1, MSJmono11_SNP1, AKMmono12_SNP1, MSJmono40_SNP1, EJmono03_SNP1 and AKMmono17v2_SNP1.

Suitably, when the method comprises the step of determining the nucleotide sequence of or more microsatellite markers, the one or more microsatellite markers may be selected from the group consisting of: EJmono12_SNP1, LMmono05v2_SNP1, AKMmono14_SNP1, MSJmono22_SNP1, EJmono14v2_SNP1, MSJmono20_SNP1, AKMmono07_SNP1, AKMmono05_SNP1, LMmono09_SNP1, AKMmono02_SNP1, AKMmono13_SNP1, LMmono08_SNP1, MSJmono39_SNP1, LMmono03_SNP1, AKMmono03_SNP1, MSJmono27_SNP1, MSJmono46_SNP1, MSJmono11_SNP1, AKMmono12_SNP1, MSJmono40_SNP1, EJmono03_SNP1, AKMmono17v2_SNP1 and AKMmono16_SNP1.

Suitably, the method comprises determining the nucleotide sequence of one or more microsatellite marker selected from the group consisting of EJmono12_SNP1, LMmono05v2_SNP1, AKMmono14_SNP1, MSJmono22_SNP1, EJmono14v2_SNP1, MSJmono20_SNP1, AKMmono07_SNP1, AKMmono05_SNP1, LMmono09_SNP1, AKMmono02_SNP1, AKMmono13_SNP1, LMmono08_SNP1, MSJmono39_SNP1, LMmono03_SNP1, AKMmono03_SNP1, MSJmono27_SNP1, MSJmono46_SNP1, MSJmono11_SNP1, AKMmono12_SNP1, MSJmono40_SNP1, EJmono03_SNP1, AKMmono17v2_SNP1, AKMmono16_SNP1 and LMmono10v2_SNP1.

Suitably, the method comprises determining the nucleotide sequence of one or more microsatellite marker selected from the group consisting AKMmono02_SNP1, AKMmono03_SNP1, AKMmono04_SNP1, AKMmono07_SNP1, AKMmono12_SNP1, AKMmono13_SNP1, AKMmono16_SNP1, EJmono12_SNP1, MSJmono20_SNP1, MSJmono39_SNP1, and MSJmono45_SNP1. Optionally the method may further comprise determining the nucleotide sequence of one or more microsatellite marker selected from the group consisting LR36, GM07, and LR44.

Suitably, the methods of the invention may comprise determining and comparing the nucleotide sequence of one or more microsatellite marker (for example 2, 3, 4, 5, 6, 7, 8, 9, 10, 11, 12 or more) selected from Table A, B, C, D, E, H, or I in combination with one or more marker described in WO2021019197, which is incorporated herein by reference. More suitably, the one or more marker selected from WO2021019197 may be selected from the group consisting of LR36, GM07, LR48, LR44, and LR52 (the details of which are provided in Table G hereinbelow), more suitably LR36, GM07, and LR44. An example of such a suitable combination of markers is shown in Table I. Additionally or alternatively, the methods of the invention may comprise determining and comparing the nucleotide sequence of one or more microsatellite marker (for example 2, 3, 4, 5, 6, 7, 8, 9, 10, 11, 12 or more) selected from Table A, B, C, D, E, H, or I in combination with the nucleotide sequence of one or more tumour mutation hotspots. Exemplary tumour mutation hotspots are provided in the Examples section of the present application. These hotspots may be particularly relevant in the context of CRC. Other suitable more tumour mutation hotspots will be known to those skilled in the art. Suitable tumour mutation hotspots are, for example, described in Modest et al., 2016 (doi: 10.1093/annonc/mdw261), which is incorporated herein by reference.

Suitably, the methods of the present invention may involve determining the nucleotide sequence of less than 63, less than 62, less than 61, less than 60, less than 59, less than 58, less than 57, less than 56, less than 55, less than 54, less than 53, less than 52, less than 51, less than 50, less than 49, less than 48, less than 47, less than 46, less than 45, less than 44, less than 43, less than 42, less than 41, less than 40, less than 39, less than 38, less than 37, less than 36, less than 35, less than 34, less than 33, less than 32, less than 31, less than 30, less than 29, less than 28, less than 27, less than 26, less than 25, less than 24, less than 23, less than 22, less than 21, less than 20, less than 19, less than 18, less than 17, less than 16, less than 15, less than 14, less than 13, less than 12, less than 11, less than 10, less than 9, less than 8, less than 7, less than 6, less than 5, less than 4, or less than 3 microsatellite markers. For avoidance of doubt, when the method of the invention comprises determining the nucleotide sequence of, for example, less than 6 microsatellite markers, it may involve one or more, but less than 6 microsatellite markers (so for example 1, 2, 3, 4, or 5 microsatellite markers).

Although the markers disclosed herein may provide accurate differentiation between MSI and MSS when analysed individually, it shall be appreciated by a person of skill in the art, the addition of further microsatellite markers may further improve accuracy and/or robustness of the methods of the invention. It will also be appreciated by a person of skill in the art, that some microsatellite markers and/or microsatellite marker combinations may be more informative than others. Tables B, C and D provide lists of markers that have been ranked from the most to least informative. It will be therefore understood by a person skilled in the art, that markers more highly ranked and/or combinations of more highly ranked markers may be more informative than markers or combinations with lower rankings.

Advantageously, the markers and/or marker combinations provided herein allow for an MSI classification accuracy of at least 0.9, preferably at least 0.95, more preferably at least 0.999 or 1. The marker combinations provided herein can therefore achieve a clinically acceptable MSI classification accuracy with significantly fewer markers than was previously understood to be necessary, meaning that the associated methods and kits can be significantly cheaper and more efficient. The marker combinations provided herein are therefore particularly advantageous in achieving a clinically acceptable MSI classification accuracy.

As mentioned herein above, in some embodiments, the methods described herein may be performed using a multiplex PCR method (for example a single-round or two-round multiplex PCR method). Such a multiplex PCR may be utilised in the amplification step of one or more markers (such as those listed in Table I) from the sample to generate microsatellite markers amplicons prior to step a).

The term “about” as used herein, for example with reference to the thermocycler programme, means plus or minus 10% or less. For example, plus or minus 9%, plus or minus 8%, plus or minus 7%, plus or minus 6%, or less. For example, plus or minus 5%, plus or minus 4%, plus or minus 3%, plus or minus 2%, or plus or minus 1%, or less.

The methods described herein may include the step of determining allelic imbalance. Assessing whether length variants are concentrated in sequence reads from one SNP allele offers an additional criterion to differentiate between PCR artefacts and mutations that occur in vivo, and can provide additional discrimination between MSI and MSS samples. This is because PCR artefacts are likely to affect both alleles equally, whereas microsatellite instability is a stochastic event affecting a single allele at a time. This can lead to bias in the levels of instability observed between the alleles at a single microsatellite marker, even if both are unstable. As mentioned elsewhere in the present specification, some of the novel markers identified by the inventors and listed in Table A are associated with SNPs. These markers may be useful in the context of a method for evaluating the biological significance of any microsatellite instability in the sample, the method comprising amplifying both the microsatellite marker and an SNP within a short distance of it in a single amplicon (by e.g. using primers and/or probes), and for heterozygous SNPs, determining whether there is a bias between indel frequencies for the two alleles of the sample.

Accordingly, in a further aspect, provided herein is a method for evaluating the biological significance of sequence variation identified during sequencing, comprising:
a) amplifying from the sample one or more microsatellite marker listed in Table E to generate microsatellite markers amplicons, wherein each microsatellite loci has a single nucleotide polymorphism (SNP) within a short distance of the microsatellite marker and said amplifying step amplifies both the microsatellite marker and associated SNP in a single amplicon;
b) sequencing the amplicons; and
c) comparing the sequences from the amplicons to predetermined sequences and determining any deviation, indicative of instability, from the predetermined sequences; and
d) for heterozygous SNPs, determining whether there is a bias between indel frequencies for the two alleles.

Suitably, the one or more microsatellite marker may be any 1, 2, 3, 4, 5, 6, 7, 8, 9, 10, 11, 12, 13 or all 14 markers in Table E. Suitably, at least one of the markers selected from Table E may be AKMmono10v2 or LMmono05v2.

Suitably, the SNP is within 100 base pairs, more suitably within 50 base pairs, most suitably within 30 base pairs from the microsatellite marker.

It will be appreciated that the embodiments described herein in the context of a method for evaluating microsatellite instability may equally apply to the method for evaluating the biological significance of sequence variation.

A method as above may be useful for identifying mismatch repair defects, wherein deviation from the predetermined sequences for one or more (for example 2, 3, 4, 5, 6 or more) microsatellite markers is indicative of a mismatch repair defect.

A method as above may be useful for identifying MSI, wherein deviation from the predetermined sequences for one or more (for example 2, 3, 4, 5, 6 or more) microsatellite markers is indicative of the sample having MSI.

In a further aspect, the invention provides a kit for use in the methods of the invention. The kit may comprise primers and/or probes for amplifying microsatellite markers and/or microsatellite markers with their associated SNPs in accordance with the above.

The kit may also comprise a thermostable polymerase and/or labelled dNTPs or analogs thereof. The labelled dNTPs or analogs thereof may be fluorescently labelled. Suitably, the kit may comprise, as well as the primers and/or probes for amplifying the microsatellite markers and/or microsatellite markers with their associated SNPs, reagents necessary for carrying out the methods of the invention, for example enzymes, dNTP mixes, buffers, PCR reaction mixes, chelating agents and/or nuclease-free water. The kit may comprise instructions for carrying out a method of the invention.

The primers and/or probes for amplifying microsatellite markers and/or microsatellite markers with their associated SNPs in accordance with the above may have sequences as provided in Table F and/or I. The kit may comprise primers and/or probes for amplifying one or more microsatellite markers and/or microsatellite markers with their associated SNPs listed in Table A. Suitably, the kit may comprise primers and/or probes for amplifying 2, 3, 4, 5, 6, 7, 8, 9, 10, 11, 12, 13, 14, 15, 16, 17, 18, 19, 20, 21, 22, 23, 24 or microsatellite markers and/or microsatellite markers with their associated SNPs listed in Table A. Suitably, the kit may comprise primers and/or probes for amplifying combinations of microsatellite markers provided elsewhere in the present specification (for example Table F). Suitably, the kit may comprise primers and/or probes for amplifying combinations of markers provided elsewhere in the present specification (for example Table I and/or Table 4).

Suitably, the kit may be kit comprising reagents necessary for carrying out a single-round multiplex PCR reaction. Suitably, such a kit may comprise a buffer (for example 5×HS VeriFi Buffer), a polymerase (for example HS VeriFi DNA Polymerase), and optionally a multiplex primer mix and/or molecular grade H₂O. Suitably the primer mix may comprise or consist of one or more primers listed in Table I and/or Table 4.

It will be appreciated that depending on the location of the microsatellite markers, it may be that more than one microsatellite marker (optionally with an associated SNP) may be amplified in a single amplicon. Typically this may be the case for markers that found within close vicinity of one another. Thus more than one marker (optionally with an associated SNP) may be amplified using one or a pair of the same probes/primers.

Throughout the present disclosure marker names such as “EJmono12_SNP1” and “EJmono12” refer to the same marker. In the tables below “rsXXXXXXX” indicates that there is no SNP associated with the marker.

TABLE A
62 Microsatellite Instability Markers

| Microsatellite marker | | Amplicon | Amplicon | MS | MS | | | | SNP |
|---|---|---|---|---|---|---|---|---|---|
| (MS) name | Chr | Start | End | Length | Base | MS Start | MS End | SNP | Position |
| EJmono12_SNP1 | 3 | 65336077 | 65336176 | 12 | A | 65336135 | 65336146 | rsxxxxxx | |
| LMmono05v2_SNP1 | 20 | 33745025 | 33745178 | 13 | A | 33745137 | 33745149 | rs6088734 | 33745046 |
| AKMmono14_SNP1 | 2 | 115319177 | 115319276 | 11 | T | 115319229 | 115319239 | rsxxxxxx | |
| MSJmono22_SNP1 | 2 | 70463295 | 70463394 | 13 | T | 70463335 | 70463347 | rsxxxxxx | |
| EJmono14v2_SNP1 | 5 | 103200164 | 103200323 | 11 | T | 103200293 | 103200303 | rs2562279 | 103200197 |
| MSJmono20_SNP1 | 8 | 142928032 | 142928131 | 13 | A | 142928089 | 142928101 | rsxxxxxx | |
| AKMmono07_SNP1 | 5 | 14855923 | 14856022 | 13 | A | 14855958 | 14855970 | rsxxxxxx | |
| AKMmono05_SNP1 | 3 | 47153191 | 47153290 | 13 | A | 47153240 | 47153252 | rsxxxxxx | |
| LMmono09_SNP1 | 19 | 49540345 | 49540444 | 14 | A | 49540384 | 49540397 | rsxxxxxx | |
| AKMmono02_SNP1 | 1 | 231388403 | 231388502 | 12 | T | 231388450 | 231388461 | rsxxxxxx | |
| AKMmono13_SNP1 | 2 | 95829866 | 95829965 | 12 | A | 95829922 | 95829933 | rsxxxxxx | |
| LMmono08_SNP1 | 15 | 73175032 | 73175131 | 11 | T | 73175070 | 73175080 | rsxxxxxx | |
| MSJmono39_SNP1 | 15 | 68400111 | 68400210 | 11 | A | 68400135 | 68400145 | rsxxxxxx | |
| LMmono03_SNP1 | 17 | 58399165 | 58399264 | 14 | T | 58399233 | 58399246 | rsxxxxxx | |
| AKMmono03_SNP1 | 2 | 20514692 | 20514791 | 13 | A | 20514738 | 20514750 | rsxxxxxx | |
| MSJmono27_SNP1 | 10 | 21613127 | 21613226 | 32 | T(C) | 21613158 | 21613189 | rsxxxxxx | |
| MSJmono46_SNP1 | 19 | 38154487 | 38154606 | 13 | A | 38154539 | 38154551 | rsxxxxxx | |
| MSJmono11_SNP1 | 1 | 235744774 | 235744873 | 19 | A(G) | 235744815 | 235744833 | rsxxxxxx | |
| AKMmono12_SNP1 | 1 | 220008858 | 220008957 | 12 | A | 220008901 | 220008912 | rsxxxxxx | |
| MSJmono40_SNP1 | 12 | 29802947 | 29803066 | 31 | T(C) | 29803003 | 29803033 | rsxxxxxx | |
| EJmono03_SNP1 | 8 | 53511183 | 53511282 | 11 | A | 53511204 | 53511214 | rsxxxxxx | |
| AKMmono17v2_SNP1 | 8 | 59843771 | 59843924 | 11 | A | 59843890 | 59843900 | rs7834158 | 59843813 |
| AKMmono16_SNP1 | 2 | 146400045 | 146400144 | 12 | A | 146400083 | 146400094 | rsxxxxxx | |
| LMmono10v2_SNP1 | 20 | 13814213 | 13814366 | 13 | T | 13814245 | 13814257 | rs10485769 | 13814346 |
| AKMmono01v2_SNP1 | 1 | 163944612 | 163944731 | 12 | T | 163944658 | 163944669 | rs12034420 | 163944712 |
| AKMmono04_SNP1 | 2 | 48427427 | 48427526 | 13 | A | 48427470 | 48427482 | rsxxxxxx | |
| AKMmono06_SNP1 | 4 | 31266091 | 31266190 | 15 | T | 31266124 | 31266138 | rsxxxxxx | |
| AKMmono08v2_SNP1 | 6 | 119660737 | 119660901 | 11 | T | 119660766 | 119660776 | rs195082 | 119660876 |
| AKMmono10v2_SNP1 | 7 | 8110742 | 8110881 | 11 | T | 8110837 | 8110847 | rs10486207 | 8110780 |
| AKMmono11_SNP1 | 1 | 188783721 | 188783820 | 12 | A | 188783747 | 188783758 | rsxxxxxx | |
| AKMmono22_SNP1 | 9 | 104829924 | 104830023 | 14 | A | 104829958 | 104829971 | rsxxxxxx | |
| EJmono01_SNP1 | 7 | 118718289 | 118718388 | 11 | A | 118718316 | 118718326 | rsxxxxxx | |
| EJmono02_SNP1 | 8 | 28204414 | 28204513 | 13 | T | 28204461 | 28204473 | rsxxxxxx | |
| EJmono04_SNP1 | 9 | 8807552 | 8807651 | 15 | T | 8807599 | 8807613 | rsxxxxxx | |
| EJmono05_SNP1 | 9 | 28483519 | 28483618 | 11 | A | 28483545 | 28483555 | rsxxxxxx | |
| EJmono06v2_SNP1 | 9 | 84371890 | 84371989 | 16 | T | 84371946 | 84371961 | rs1007995 | 84371927 |
| EJmono13v2_SNP1 | 5 | 4294916 | 4295069 | 11 | T | 4294954 | 4294964 | rs16873198 | 4295050 |
| EJmono16_SNP1 | 10 | 57222808 | 57222907 | 13 | A | 57222859 | 57222871 | rsxxxxxx | |
| EJmono21v2_SNP1 | 18 | 7068682 | 7068835 | 11 | A | 7068797 | 7068807 | rs7234998 | 7068724 |
| HGtetra23ms2_SNP1 | 10 | 70692765 | 70692864 | 14 | T | 70692828 | 70692841 | rsxxxxxx | |
| LMmono01_SNP1 | 14 | 89137182 | 89137281 | 11 | A | 89137211 | 89137221 | rsxxxxxx | |
| LMmono04v2_SNP1 | 18 | 53192420 | 53192519 | 13 | A | 53192468 | 53192480 | rs10401120 | 53192498 |
| LMmono07_SNP1 | 15 | 55347955 | 55348054 | 13 | A | 55347997 | 55348009 | rsxxxxxx | |
| LMmono12_SNP1 | 16 | 80521224 | 80521323 | 13 | T | 80521284 | 80521296 | rsxxxxxx | |
| LMmono16_SNP1 | 1 | 150737519 | 150737618 | 15 | A | 150737578 | 150737592 | rsxxxxxx | |
| MSJcom06ms1_SNP1 | 10 | 105386332 | 105386431 | 13 | A | 105386364 | 105386376 | rsxxxxxx | |
| MSJcom06ms2_SNP1 | 10 | 105386332 | 105386431 | 13 | A | 105386384 | 105386396 | rsxxxxxx | |
| MSJmono10_SNP1 | 1 | 219386763 | 219386862 | 12 | A | 219386785 | 219386796 | rsxxxxxx | |
| MSJmono15_SNP1 | 16 | 50022742 | 50022841 | 13 | A | 50022767 | 50022779 | rsxxxxxx | |
| MSJmono17_SNP1 | 16 | 34332329 | 34332428 | 21 | T(G) | 34332376 | 34332396 | rsxxxxxx | |
| MSJmono19ms1_SNP1 | 12 | 80004517 | 80004616 | 25 | A(G) | 80004543 | 80004567 | rsxxxxxx | |
| MSJmono19ms2_SNP1 | 12 | 80004517 | 80004616 | 11 | A | 80004578 | 80004588 | rsxxxxxx | |
| MSJmono23v2_SNP1 | 6 | 126371727 | 126371886 | 13 | A | 126371845 | 126371857 | rs4897168 | 126371788 |
| MSJmono26_SNP1 | 14 | 104291000 | 104291099 | 14 | A | 104291032 | 104291045 | rsxxxxxx | |
| MSJmono30v2_SNP1 | 4 | 140348762 | 140348915 | 13 | T | 140348882 | 140348894 | rs13136124 | 140348815 |
| MSJmono32_SNP1 | 4 | 75787256 | 75787395 | 13 | A | 75787356 | 75787368 | rs7700246 | 75787289 |
| MSJmono36_SNP1 | 15 | 56017765 | 56017864 | 13 | A | 56017792 | 56017804 | rsxxxxxx | |
| MSJmono37_SNP1 | 14 | 64159139 | 64159238 | 12 | T | 64159174 | 64159185 | rsxxxxxx | |
| MSJmono38_SNP1 | 13 | 59791849 | 59791948 | 12 | A | 59791883 | 59791894 | rsxxxxxx | |
| MSJmono41_SNP1 | 12 | 50382548 | 50382647 | 20 | T(C) | 50382596 | 50382615 | rsxxxxxx | |
| MSJmono44_SNP1 | 4 | 1911304 | 1911423 | 16 | T(C) | 1911366 | 1911381 | rsxxxxxx | |
| MSJmono45_SNP1 | 6 | 14647951 | 14648050 | 25 | T(C) | 14647998 | 14648022 | rsxxxxxx | |

TABLE B
28 microsatellite instability markers

| | Microsatellite marker | | Amplicon | Amplicon | MS | MS | | | | SNP |
|---|---|---|---|---|---|---|---|---|---|---|
| Rank | (MS) name | Chr | Start | End | Length | Base | MS Start | MS End | SNP | Position |
| 1 | AKMmono10v2_SNP1 | 7 | 8110742 | 8110881 | 11 | T | 8110837 | 8110847 | rs10486207 | 8110780 |
| 2 | LMmono05v2_SNP1 | 20 | 33745025 | 33745178 | 13 | A | 33745137 | 33745149 | rs6088734 | 33745046 |
| 3 | AKMmono05_SNP1 | 3 | 47153191 | 47153290 | 13 | A | 47153240 | 47153252 | rsxxxxxx | |
| 4 | AKMmono17v2_SNP1 | 8 | 59843771 | 59843924 | 11 | A | 59843890 | 59843900 | rs7834158 | 59843813 |
| 5 | LMmono09_SNP1 | 19 | 49540345 | 49540444 | 14 | A | 49540384 | 49540397 | rsxxxxxx | |
| 6 | LMmono03_SNP1 | 17 | 58399165 | 58399264 | 14 | T | 58399233 | 58399246 | rsxxxxxx | |
| 7 | LMmono10v2_SNP1 | 20 | 13814213 | 13814366 | 13 | T | 13814245 | 13814257 | rs10485769 | 13814346 |
| 8 | LMmono04v2_SNP1 | 18 | 53192420 | 53192519 | 13 | A | 53192468 | 53192480 | rs10401120 | 53192498 |
| 9 | AKMmono13_SNP1 | 2 | 95829866 | 95829965 | 12 | A | 95829922 | 95829933 | rsxxxxxx | |
| 10 | AKMmono16_SNP1 | 2 | 146400045 | 146400144 | 12 | A | 146400083 | 146400094 | rsxxxxxx | |
| 11 | EJmono13v2_SNP1 | 5 | 4294916 | 4295069 | 11 | T | 4294954 | 4294964 | rs16873198 | 4295050 |
| 12 | AKMmono02_SNP1 | 1 | 231388403 | 231388502 | 12 | T | 231388450 | 231388461 | rsxxxxxx | |
| 13 | EJmono16_SNP1 | 10 | 57222808 | 57222907 | 13 | A | 57222859 | 57222871 | rsxxxxxx | |
| 14 | LMmono01_SNP1 | 14 | 89137182 | 89137281 | 11 | A | 89137211 | 89137221 | rsxxxxxx | |
| 15 | AKMmono03_SNP1 | 2 | 20514692 | 20514791 | 13 | A | 20514738 | 20514750 | rsxxxxxx | |
| 16 | EJmono21v2_SNP1 | 18 | 7068682 | 7068835 | 11 | A | 7068797 | 7068807 | rs7234998 | 7068724 |
| 17 | MSJmono30v2_SNP1 | 4 | 140348762 | 140348915 | 13 | T | 140348882 | 140348894 | rs13136124 | 140348815 |
| 18 | MSJmono46_SNP1 | 19 | 38154487 | 38154606 | 13 | A | 38154539 | 38154551 | rsxxxxxx | |
| 19 | MSJmono10_SNP1 | 1 | 219386763 | 219386862 | 12 | A | 219386785 | 219386796 | rsxxxxxx | |
| 20 | LMmono16_SNP1 | 1 | 150737519 | 150737618 | 15 | A | 150737578 | 150737592 | rsxxxxxx | |
| 21 | MSJmono44_SNP1 | 4 | 1911304 | 1911423 | 16 | T(C) | 1911366 | 1911381 | rsxxxxxx | |
| 22 | AKMmono08v2_SNP1 | 6 | 119660737 | 119660901 | 11 | T | 119660766 | 119660776 | rs195082 | 119660876 |
| 23 | EJmono14v2_SNP1 | 5 | 103200164 | 103200323 | 11 | T | 103200293 | 103200303 | rs2562279 | 103200197 |
| 24 | LMmono12_SNP1 | 16 | 80521224 | 80521323 | 13 | T | 80521284 | 80521296 | rsxxxxxx | |
| 25 | EJmono05_SNP1 | 9 | 28483519 | 28483618 | 11 | A | 28483545 | 28483555 | rsxxxxxx | |
| 26 | MSJmono19ms2_SNP1 | 12 | 80004517 | 80004616 | 11 | A | 80004578 | 80004588 | rsxxxxxx | |
| 27 | AKMmono12_SNP1 | 1 | 220008858 | 220008957 | 12 | A | 220008901 | 220008912 | rsxxxxxx | |
| 28 | MSJmono19ms1_SNP1 | 12 | 80004517 | 80004616 | 25 | A(G) | 80004543 | 80004567 | rsxxxxxx | |

TABLE C
54 Microsatellite Instability Markers

| | Microsatellite marker | | Amplicon | Amplicon | MS | MS | | | | SNP |
|---|---|---|---|---|---|---|---|---|---|---|
| Rank | (MS) name | Chr | Start | End | Length | Base | MS Start | MS End | SNP | Position |
| 1 | EJmono12_SNP1 | 3 | 65336077 | 65336176 | 12 | A | 65336135 | 65336146 | rsxxxxxx | |
| 2 | LMmono05v2_SNP1 | 20 | 33745025 | 33745178 | 13 | A | 33745137 | 33745149 | rs6088734 | 33745046 |
| 3 | AKMmono14_SNP1 | 2 | 115319177 | 115319276 | 11 | T | 115319229 | 115319239 | rsxxxxxx | |
| 4 | MSJmono22_SNP1 | 2 | 70463295 | 70463394 | 13 | T | 70463335 | 70463347 | rsxxxxxx | |
| 5 | EJmono14v2_SNP1 | 5 | 103200164 | 103200323 | 11 | T | 103200293 | 103200303 | rs2562279 | 103200197 |
| 6 | MSJmono20_SNP1 | 8 | 142928032 | 142928131 | 13 | A | 142928089 | 142928101 | rsxxxxxx | |
| 7 | AKMmono07_SNP1 | 5 | 14855923 | 14856022 | 13 | A | 14855958 | 14855970 | rsxxxxxx | |
| 8 | AKMmono05_SNP1 | 3 | 47153191 | 47153290 | 13 | A | 47153240 | 47153252 | rsxxxxxx | |
| 9 | LMmono09_SNP1 | 19 | 49540345 | 49540444 | 14 | A | 49540384 | 49540397 | rsxxxxxx | |
| 10 | AKMmono02_SNP1 | 1 | 231388403 | 231388502 | 12 | T | 231388450 | 231388461 | rsxxxxxx | |
| 11 | AKMmono13_SNP1 | 2 | 95829866 | 95829965 | 12 | A | 95829922 | 95829933 | rsxxxxxx | |
| 12 | LMmono08_SNP1 | 15 | 73175032 | 73175131 | 11 | T | 73175070 | 73175080 | rsxxxxxx | |
| 13 | MSJmono39_SNP1 | 15 | 68400111 | 68400210 | 11 | A | 68400135 | 68400145 | rsxxxxxx | |
| 14 | LMmono03_SNP1 | 17 | 58399165 | 58399264 | 14 | T | 58399233 | 58399246 | rsxxxxxx | |
| 15 | AKMmono03_SNP1 | 2 | 20514692 | 20514791 | 13 | A | 20514738 | 20514750 | rsxxxxxx | |
| 16 | MSJmono27_SNP1 | 10 | 21613127 | 21613226 | 32 | T(C) | 21613158 | 21613189 | rsxxxxxx | |
| 17 | MSJmono46_SNP1 | 19 | 38154487 | 38154606 | 13 | A | 38154539 | 38154551 | rsxxxxxx | |
| 18 | MSJmono11_SNP1 | 1 | 235744774 | 235744873 | 19 | A(G) | 235744815 | 235744833 | rsxxxxxx | |
| 19 | AKMmono12_SNP1 | 1 | 220008858 | 220008957 | 12 | A | 220008901 | 220008912 | rsxxxxxx | |
| 20 | MSJmono40_SNP1 | 12 | 29802947 | 29803066 | 31 | T(C) | 29803003 | 29803033 | rsxxxxxx | |
| 21 | EJmono03_SNP1 | 8 | 53511183 | 53511282 | 11 | A | 53511204 | 53511214 | rsxxxxxx | |
| 22 | AKMmono17v2_SNP1 | 8 | 59843771 | 59843924 | 11 | A | 59843890 | 59843900 | rs7834158 | 59843813 |
| 23 | AKMmono16_SNP1 | 2 | 146400045 | 146400144 | 12 | A | 146400083 | 146400094 | rsxxxxxx | |
| 24 | LMmono10v2_SNP1 | 20 | 13814213 | 13814366 | 13 | T | 13814245 | 13814257 | rs10485769 | 13814346 |
| 25 | AKMmono10v2_SNP1 | 7 | 8110742 | 8110881 | 11 | T | 8110837 | 8110847 | rs10486207 | 8110780 |
| 26 | MSJmono10_SNP1 | 1 | 219386763 | 219386862 | 12 | A | 219386785 | 219386796 | rsxxxxxx | |
| 27 | EJmono05_SNP1 | 9 | 28483519 | 28483618 | 11 | A | 28483545 | 28483555 | rsxxxxxx | |
| 28 | LMmono12_SNP1 | 16 | 80521224 | 80521323 | 13 | T | 80521284 | 80521296 | rsxxxxxx | |
| 29 | EJmono06v2_SNP1 | 9 | 84371890 | 84371989 | 16 | T | 84371946 | 84371961 | rs1007995 | 84371927 |
| 30 | EJmono01_SNP1 | 7 | 118718289 | 118718388 | 11 | A | 118718316 | 118718326 | rsxxxxxx | |
| 31 | MSJmono23v2_SNP1 | 6 | 126371727 | 126371886 | 13 | A | 126371845 | 126371857 | rs4897168 | 126371788 |
| 32 | AKMmono11_SNP1 | 1 | 188783721 | 188783820 | 12 | A | 188783747 | 188783758 | rsxxxxxx | |
| 33 | EJmono16_SNP1 | 10 | 57222808 | 57222907 | 13 | A | 57222859 | 57222871 | rsxxxxxx | |
| 34 | MSJmono44_SNP1 | 4 | 1911304 | 1911423 | 16 | T(C) | 1911366 | 1911381 | rsxxxxxx | |
| 35 | MSJmono38_SNP1 | 13 | 59791849 | 59791948 | 12 | A | 59791883 | 59791894 | rsxxxxxx | |
| 36 | MSJmono19ms2_SNP1 | 12 | 80004517 | 80004616 | 11 | A | 80004578 | 80004588 | rsxxxxxx | |
| 37 | MSJmono41_SNP1 | 12 | 50382548 | 50382647 | 20 | T(C) | 50382596 | 50382615 | rsxxxxxx | |
| 38 | MSJmono26_SNP1 | 14 | 104291000 | 104291099 | 14 | A | 104291032 | 104291045 | rsxxxxxx | |
| 39 | LMmono01_SNP1 | 14 | 89137182 | 89137281 | 11 | A | 89137211 | 89137221 | rsxxxxxx | |
| 40 | MSJmono30v2_SNP1 | 4 | 140348762 | 140348915 | 13 | T | 140348882 | 140348894 | rs13136124 | 140348815 |
| 41 | MSJmono37_SNP1 | 14 | 64159139 | 64159238 | 12 | T | 64159174 | 64159185 | rsxxxxxx | |
| 42 | LMmono16_SNP1 | 1 | 150737519 | 150737618 | 15 | A | 150737578 | 150737592 | rsxxxxxx | |
| 43 | EJmono02_SNP1 | 8 | 28204414 | 28204513 | 13 | T | 28204461 | 28204473 | rsxxxxxx | |
| 44 | AKMmono08v2_SNP1 | 6 | 119660737 | 119660901 | 11 | T | 119660766 | 119660776 | rs195082 | 119660876 |
| 45 | LMmono04v2_SNP1 | 18 | 53192420 | 53192519 | 13 | A | 53192468 | 53192480 | rs10401120 | 53192498 |
| 46 | EJmono13v2_SNP1 | 5 | 4294916 | 4295069 | 11 | T | 4294954 | 4294964 | rs16873198 | 4295050 |
| 47 | MSJmono15_SNP1 | 16 | 50022742 | 50022841 | 13 | A | 50022767 | 50022779 | rsxxxxxx | |
| 48 | LMmono07_SNP1 | 15 | 55347955 | 55348054 | 13 | A | 55347997 | 55348009 | rsxxxxxx | |
| 49 | AKMmono06_SNP1 | 4 | 31266091 | 31266190 | 15 | T | 31266124 | 31266138 | rsxxxxxx | |
| 50 | MSJmono36_SNP1 | 15 | 56017765 | 56017864 | 13 | A | 56017792 | 56017804 | rsxxxxxx | |
| 51 | MSJmono19ms1_SNP1 | 12 | 80004517 | 80004616 | 25 | A(G) | 80004543 | 80004567 | rsxxxxxx | |
| 52 | AKMmono22_SNP1 | 9 | 104829924 | 104830023 | 14 | A | 104829958 | 104829971 | rsxxxxxx | |
| 53 | AKMmono01v2_SNP1 | 1 | 163944612 | 163944731 | 12 | T | 163944658 | 163944669 | rs12034420 | 163944712 |
| 54 | EJmono21v2_SNP1 | 18 | 7068682 | 7068835 | 11 | A | 7068797 | 7068807 | rs7234998 | 7068724 |

TABLE D
24 Microsatellite Instability Markers

| | Microsatellite marker | | Amplicon | Amplicon | MS | MS | | | | SNP |
|---|---|---|---|---|---|---|---|---|---|---|
| Rank | (MS) name | Chr | Start | End | Length | Base | MS Start | MS End | SNP | Position |
| 1 | EJmono12_SNP1 | 3 | 65336077 | 65336176 | 12 | A | 65336135 | 65336146 | rsxxxxxx | |
| 2 | LMmono05v2_SNP1 | 20 | 33745025 | 33745178 | 13 | A | 33745137 | 33745149 | rs6088734 | 33745046 |
| 3 | AKMmono14_SNP1 | 2 | 115319177 | 115319276 | 11 | T | 115319229 | 115319239 | rsxxxxxx | |
| 4 | MSJmono22_SNP1 | 2 | 70463295 | 70463394 | 13 | T | 70463335 | 70463347 | rsxxxxxx | |
| 5 | EJmono14v2_SNP1 | 5 | 103200164 | 103200323 | 11 | T | 103200293 | 103200303 | rs2562279 | 103200197 |
| 6 | MSJmono20_SNP1 | 8 | 142928032 | 142928131 | 13 | A | 142928089 | 142928101 | rsxxxxxx | |
| 7 | AKMmono07_SNP1 | 5 | 14855923 | 14856022 | 13 | A | 14855958 | 14855970 | rsxxxxxx | |
| 8 | AKMmono05_SNP1 | 3 | 47153191 | 47153290 | 13 | A | 47153240 | 47153252 | rsxxxxxx | |
| 9 | LMmono09_SNP1 | 19 | 49540345 | 49540444 | 14 | A | 49540384 | 49540397 | rsxxxxxx | |
| 10 | AKMmono02_SNP1 | 1 | 231388403 | 231388502 | 12 | T | 231388450 | 231388461 | rsxxxxxx | |
| 11 | AKMmono13_SNP1 | 2 | 95829866 | 95829965 | 12 | A | 95829922 | 95829933 | rsxxxxxx | |
| 12 | LMmono08_SNP1 | 15 | 73175032 | 73175131 | 11 | T | 73175070 | 73175080 | rsxxxxxx | |
| 13 | MSJmono39_SNP1 | 15 | 68400111 | 68400210 | 11 | A | 68400135 | 68400145 | rsxxxxxx | |
| 14 | LMmono03_SNP1 | 17 | 58399165 | 58399264 | 14 | T | 58399233 | 58399246 | rsxxxxxx | |
| 15 | AKMmono03_SNP1 | 2 | 20514692 | 20514791 | 13 | A | 20514738 | 20514750 | rsxxxxxx | |
| 16 | MSJmono27_SNP1 | 10 | 21613127 | 21613226 | 32 | T(C) | 21613158 | 21613189 | rsxxxxxx | |
| 17 | MSJmono46_SNP1 | 19 | 38154487 | 38154606 | 13 | A | 38154539 | 38154551 | rsxxxxxx | |
| 18 | MSJmono11_SNP1 | 1 | 235744774 | 235744873 | 19 | A(G) | 235744815 | 235744833 | rsxxxxxx | |
| 19 | AKMmono12_SNP1 | 1 | 220008858 | 220008957 | 12 | A | 220008901 | 220008912 | rsxxxxxx | |
| 20 | MSJmono40_SNP1 | 12 | 29802947 | 29803066 | 31 | T(C) | 29803003 | 29803033 | rsxxxxxx | |
| 21 | EJmono03_SNP1 | 8 | 53511183 | 53511282 | 11 | A | 53511204 | 53511214 | rsxxxxxx | |
| 22 | AKMmono17v2_SNP1 | 8 | 59843771 | 59843924 | 11 | A | 59843890 | 59843900 | rs7834158 | 59843813 |
| 23 | AKMmono16_SNP1 | 2 | 146400045 | 146400144 | 12 | A | 146400083 | 146400094 | rsxxxxxx | |
| 24 | LMmono10v2_SNP1 | 20 | 13814213 | 13814366 | 13 | T | 13814245 | 13814257 | rs10485769 | 13814346 |

TABLE E
Microsatellite Markers associated with SNP

| Microsatellite | | Amplicon | Amplicon | MS | MS | | | | SNP |
|---|---|---|---|---|---|---|---|---|---|
| (MS) name | Chr | Start | End | Length | Base | MS Start | MS End | SNP | Position |
| LMmono05v2_SNP1 | 20 | 33745025 | 33745178 | 13 | A | 33745137 | 33745149 | rs6088734 | 33745046 |
| EJmono14v2_SNP1 | 5 | 103200164 | 103200323 | 11 | T | 103200293 | 103200303 | rs2562279 | 103200197 |
| AKMmono17v2_SNP1 | 8 | 59843771 | 59843924 | 11 | A | 59843890 | 59843900 | rs7834158 | 59843813 |
| LMmono10v2_SNP1 | 20 | 13814213 | 13814366 | 13 | T | 13814245 | 13814257 | rs10485769 | 13814346 |
| AKMmono01v2_SNP1 | 1 | 163944612 | 163944731 | 12 | T | 163944658 | 163944669 | rs12034420 | 163944712 |
| AKMmono08v2_SNP1 | 6 | 119660737 | 119660901 | 11 | T | 119660766 | 119660776 | rs195082 | 119660876 |
| AKMmono10v2_SNP1 | 7 | 8110742 | 8110881 | 11 | T | 8110837 | 8110847 | rs10486207 | 8110780 |
| EJmono06v2_SNP1 | 9 | 84371890 | 84371989 | 16 | T | 84371946 | 84371961 | rs1007995 | 84371927 |
| EJmono13v2_SNP1 | 5 | 4294916 | 4295069 | 11 | T | 4294954 | 4294964 | rs16873198 | 4295050 |
| EJmono21v2_SNP1 | 18 | 7068682 | 7068835 | 11 | A | 7068797 | 7068807 | rs7234998 | 7068724 |
| LMmono04v2_SNP1 | 18 | 53192420 | 53192519 | 13 | A | 53192468 | 53192480 | rs10401120 | 53192498 |
| MSJmono23v2_SNP1 | 6 | 126371727 | 126371886 | 13 | A | 126371845 | 126371857 | rs4897168 | 126371788 |
| MSJmono30v2_SNP1 | 4 | 140348762 | 140348915 | 13 | T | 140348882 | 140348894 | rs13136124 | 140348815 |
| MSJmono32_SNP1 | 4 | 75787256 | 75787395 | 13 | A | 75787356 | 75787368 | rs7700246 | 75787289 |

TABLE F
Microsatellite Instability Marker Probe Sequences

| Marker | Sequence identification | MIP Sequence |
|---|---|---|
| AKMmono01v2 | SEQ ID NO: 1 | CCCACTCCCTGGTTTTTTAAAACTCAGAGNNNNCTTCAGCTTCCCGATATCCGACGGTAGTGTNNNNACTGAGGTAGTGTCTG |
| AKMmono02 | SEQ ID NO: 2 | GAGGAGTTTCTTAAGAATATTTTCTCTTNNNNCTTCAGCTTCCCGATATCCGACGGTAGTGTNNNNCATGCTTGTTTTCCTTT |
| AKMmono03 | SEQ ID NO: 3 | TGGGCATCAATCCTTCCAAGTCTTCNNNNCTTCAGCTTCCCGATATCCGACGGTAGTGTNNNNGGCATTCAAGTCTAACAGAG |
| AKMmono04 | SEQ ID NO: 4 | GTGTGTTTAATTGCTTTACCTTAGGGTGNNNNCTTCAGCTTCCCGATATCCGACGGTAGTGTNNNNGACACTGACCAAATTGT |
| AKMmono05 | SEQ ID NO: 5 | GCTTATCAAAAGATATATGAGGATTCCTGNNNNCTTCAGCTTCCCGATATCCGACGGTAGTGTNNNNGTTCCCAAAAGGATAG |
| AKMmono06 | SEQ ID NO: 6 | TCTATCTCTATTGGATAATCTTCAAAGACNNNNCTTCAGCTTCCCGATATCCGACGGTAGTGTNNNNCCTTTCTACTACATGC |
| AKMmono07 | SEQ ID NO: 7 | TAAAATCTAAGTGCCCCAATCATCTGTCNNNNCTTCAGCTTCCCGATATCCGACGGTAGTGTNNNNGCCTTTTCCCTAAGGTG |
| AKMmono08v2 | SEQ ID NO: 8 | ATCCTTCATAGTITTATCAGTCACTANNNNCTTCAGCTTCCCGATATCCGACGGTAGTGTNNNNCTTTGAGATTTGTCTTATC |
| AKMmono10v2 | SEQ ID NO: 9 | CATTCTTACCTTTATCACGATCATAGAGCNNNNCTTCAGCTTCCCGATATCCGACGGTAGTGTNNNNACCAATCTTTGACAAT |
| AKMmono11 | SEQ ID NO: 10 | GTCATTAACATAATGCCTCCTTATGTTCNNNNCTTCAGCTTCCCGATATCCGACGGTAGTGTNNNNGTCTTGCACATGTACCC |
| AKMmono12 | SEQ ID NO: 11 | CAGTGTAATTTGAAAACTCCAGCTGTCCANNNNCTTCAGCTTCCCGATATCCGACGGTAGTGTNNNNCTGTTTATGCAGCAAG |
| AKMmono13 | SEQ ID NO: 12 | TTCTCTTATGGATGTTGTGACTTTAATCNNNNCTTCAGCTTCCCGATATCCGACGGTAGTGTNNNNCAGTTTCCACCCTCAGT |
| AKMmono14 | SEQ ID NO: 13 | GCTAAAGGAGGAGGTGGATTTTTTCTCTNNNNCTTCAGCTTCCCGATATCCGACGGTAGTGTNNNNCTAAAATAGACTTGGAA |
| AKMmono16 | SEQ ID NO: 14 | CCTCAGTGAGACCAATAGAGCTTGTTCAGNNNNCTTCAGCTTCCCGATATCCGACGGTAGTGTNNNNGCTCCTTCACACTTGT |
| AKMmono17v2 | SEQ ID NO: 15 | GCCTCTACGTGGATGTTTTATGNNNNCTTCAGCTTCCCGATATCCGACGGTAGTGTNNNNTCAACTTTTAAATCAAATTAG |
| AKMmono22 | SEQ ID NO: 16 | TTCCTATTCATGTGATGAGATGACTACANNNNCTTCAGCTTCCCGATATCCGACGGTAGTGTNNNNGTTGGACTTTGCCCCAG |
| EJmono01 | SEQ ID NO: 17 | CAGTAATTTCAGAGATATATAGAGAAAACNNNNCTTCAGCTTCCCGATATCCGACGGTAGTGTNNNNAGACATCAATAGCTTT |
| EJmono02 | SEQ ID NO: 18 | ACCCCATCACCAAAGACACCNNNNCTTCAGCTTCCCGATATCCGACGGTAGTGTNNNNGGCTAGCCTGGCTTTGAGCTTTACC |
| EJmono03 | SEQ ID NO: 19 | GCATTTGCTGCCTGATTCTCCTCATGTGGNNNNCTTCAGCTTCCCGATATCCGACGGTAGTGTNNNNGCCTAGGCACTGTCTC |
| EJmono04 | SEQ ID NO: 20 | CCATTCACAAATAAAATTACCCACACAGNNNNCTTCAGCTTCCCGATATCCGACGGTAGTGTNNNNGCTCTAACCAAATCCAT |
| EJmono05 | SEQ ID NO: 21 | GAGTCACATTCAGGCCTTAAAATAACTGTNNNNCTTCAGCTTCCCGATATCCGACGGTAGTGTNNNNGGTTTCACTGCTGCTT |
| EJmono06v2 | SEQ ID NO: 22 | CACTAATGAAAGGGCAATCGCTGTAGGNNNNCTTCAGCTTCCCGATATCCGACGGTAGTGTNNNNGCCTCAAGGGTTAGGCTG |
| EJmono12 | SEQ ID NO: 23 | GCTAGTAAGGTGGGAATTTGTAAACCTGTNNNNCTTCAGCTTCCCGATATCCGACGGTAGTGTNNNNAAGATGTGGTCAACTT |
| EJmono13v2 | SEQ ID NO: 24 | CGGTCTTTCCACATAGGATAATTGGGGNNNNCTTCAGCTTCCCGATATCCGACGGTAGTGTNNNNTGAACGCAAGTGGCAA |
| EJmono14v2 | SEQ ID NO: 25 | AAAGTGCAAGTAAATATTAAACAACTGGNNNNCTTCAGCTTCCCGATATCCGACGGTAGTGTNNNNCCTCAGTTCCTTCTTTA |
| EJmono16 | SEQ ID NO: 26 | ACCAACCAACAAACAAAAAGCTGAAACCNNNNCTTCAGCTTCCCGATATCCGACGGTAGTGTNNNNGTGGGTGATTGCAATTG |
| EJmono21v2 | SEQ ID NO: 27 | GCTTATAAAAACCTCAGCTAGGTCTCAGANNNNCTTCAGCTTCCCGATATCCGACGGTAGTGTNNNNTATAGTGCAGTTTGGT |
| HGtetra23ms2 | SEQ ID NO: 28 | CTTTGTACTTGTATCTCTGGATGCCNNNNCTTCAGCTTCCCGATATCCGACGGTAGTGTNNNNGGACAAGAGTGAAGCTTCAT |
| LMmono01 | SEQ ID NO: 29 | CTATGGGATTATGTAGAAAGACTGAACCNNNNCTTCAGCTTCCCGATATCCGACGGTAGTGTNNNNCTGAAATAAGATACACT |
| LMmono03 | SEQ ID NO: 30 | TAATAGAGTTGACCATCACAACGAATGGNNNNCTTCAGCTTCCCGATATCCGACGGTAGTGTNNNNGTTAGTATCACTAGGGC |
| LMmono04v2 | SEQ ID NO: 31 | TCTTCATTCCACGTAACCNNNNCTTCAGCTTCCCGATATCCGACGGTAGTGTNNNNTGCCATGTTCCAATCAATTCTAAATC |
| LMmono05v2 | SEQ ID NO: 32 | GAGTACTTACGATGTGCCAAATACNNNNCTTCAGCTTCCCGATATCCGACGGTAGTGTNNNNAGGCACAAAAAGATAAAA |
| LMmono07 | SEQ ID NO: 33 | CCCAGTCTCAACTATTATGTAATAGCAGNNNNCTTCAGCTTCCCGATATCCGACGGTAGTGTNNNNGCTAGCTCCTTGATCTT |
| LMmono08 | SEQ ID NO: 34 | TGGCTGGAAATTTTCCAAACTTGATGNNNNCTTCAGCTTCCCGATATCCGACGGTAGTGTNNNNAAGAAGCCCAATACACAGG |
| LMmono09 | SEQ ID NO: 35 | GTTGACCTCGAACTCCAGTCCNNNNCTTCAGCTTCCCGATATCCGACGGTAGTGTNNNNGCGTCCTCCCTTTATGTTTTGTTG |
| LMmono10v2 | SEQ ID NO: 36 | GAAGTGTGGGAAAACTGGCACTCTGGCNNNNCTTCAGCTTCCCGATATCCGACGGTAGTGTNNNNGAAAGCAGGAGACTACG |
| LMmono12 | SEQ ID NO: 37 | TCCACAGTGACACAAACCAATTCCNNNNCTTCAGCTTCCCGATATCCGACGGTAGTGTNNNNGATGCACTTTGAAACAGCCTT |
| LMmono16 | SEQ ID NO: 38 | CATCCTTAAGAGAGACAAACCTCTGNNNNCTTCAGCTTCCCGATATCCGACGGTAGTGTNNNNGGGACTTTCCAGGATTTGCC |
| MSJcom06ms1 | SEQ ID NO: 39 | TGGGTGCGGTGGCCCACACCTGTAATTNNNNCTTCAGCTTCCCGATATCCGACGGTAGTGTNNNNCCTCACTGAGTAGTTTTT |
| + ms2 | | |
| MSJmono10 | SEQ ID NO: 40 | GTACATCAATTTGGGGAGAATTTGCATCCNNNNCTTCAGCTTCCCGATATCCGACGGTAGTGTNNNNAAACAACCTTGTCTGT |
| MSJmono11 | SEQ ID NO: 41 | TCCCCCTTCTCTCTCTTTCTCCTGCTANNNNCTTCAGCTTCCCGATATCCGACGGTAGTGTNNNNAGCCGAGATCACACCTGG |
| MSJmono15 | SEQ ID NO: 42 | GTGAAGCCTGACCAATGAAGACATCNNNNCTTCAGCTTCCCGATATCCGACGGTAGTGTNNNNGGGCAACAGAGAGAGATGCC |
| MSJmono17 | SEQ ID NO: 43 | GTGGGTGTACTAAACATATTTGATACCTNNNNCTTCAGCTTCCCGATATCCGACGGTAGTGTNNNNTAGCTTGGGTGACGGAG |
| MSJmono19ms1 | SEQ ID NO: 44 | CACAAATTGGTAACACTGATCCATCTNNNNCTTCAGCTTCCCGATATCCGACGGTAGTGTNNNNGAGAGATTCTGTCTCTACC |
| + ms2 | | |
| MSJmono20 | SEQ ID NO: 45 | GACTTGAGGATATCCTCCAGGAAAATGNNNNCTTCAGCTTCCCGATATCCGACGGTAGTGTNNNNTAGCACTGCAGTGAGCTG |
| MSJmono22 | SEQ ID NO: 46 | GTGTTTCAGATACGTCGGTAACNNNNCTTCAGCTTCCCGATATCCGACGGTAGTGTNNNNCGTGCCATTGCACTCTATCCTGG |
| MSJmono23v2 | SEQ ID NO: 47 | TTCCCACCTCAGCCTCCTGAGTAGCTAGCNNNNCTTCAGCTTCCCGATATCCGACGGTAGTGTNNNNCACATCCTACACTCCA |
| MSJmono26 | SEQ ID NO: 48 | GCGGAGTCTCGCTCTGTCGCCCATGCTGGNNNNCTTCAGCTTCCCGATATCCGACGGTAGTGTNNNNATACCCACATGATCAT |
| MSJmono27 | SEQ ID NO: 49 | TGGTGGGATTGTTCACACCTGTAATCCCNNNNCTTCAGCTTCCCGATATCCGACGGTAGTGTNNNNACTGTAACCTGGCCAAC |
| MSJmono30v2 | SEQ ID NO: 50 | TCCTTTATAAATTACCCAGTCTCGGCCNNNNCTTCAGCTTCCCGATATCCGACGGTAGTGTNNNNAAATAAAGTGGTTAAGAA |
| MSJmono32 | SEQ ID NO: 51 | GGAGTTGCAACCCTGGTTTTAGATNNNNCTTCAGCTTCCCGATATCCGACGGTAGTGTNNNNAATCGGTGTGCTGTATTCAGG |
| MSJmono36 | SEQ ID NO: 52 | TGGCAGAAGTCCTTCCTTATCAGTANNNNCTTCAGCTTCCCGATATCCGACGGTAGTGTNNNNGTCTGGGTGAGACAGTGACC |
| MSJmono37 | SEQ ID NO: 53 | GCCTGTAGTGTAGTGGCGTGGTCATAGNNNNCTTCAGCTTCCCGATATCCGACGGTAGTGTNNNNATTGGGAGTGAAGAGAAA |
| MSJmono38 | SEQ ID NO: 54 | GCTGGGCCAGGACACTCAGAGTTCTCTCTNNNNCTTCAGCTTCCCGATATCCGACGGTAGTGTNNNNTCATCATATTCTGGTG |
| MSJmono39 | SEQ ID NO: 55 | CCAGTAAATACGATACATGGTTTGTAGGNNNNCTTCAGCTTCCCGATATCCGACGGTAGTGTNNNNAGCTAGATAAAACACAC |
| MSJmono40 | SEQ ID NO: 56 | CAGATTGAGGTACTGAATAGTTTTGNNNNCTTCAGCTTCCCGATATCCGACGGTAGTGTNNNNTCAGCCTGGGCAACAGACCG |
| MSJmono41 | SEQ ID NO: 57 | GTTCATCCCCATTTCCCANNNNCTTCAGCTTCCCGATATCCGACGGTAGTGTNNNNAGCCTGGGTGACAGAGTGTGAGACCCT |
| MSJmono44 | SEQ ID NO: 58 | TTCACAGGACCCATGCTCAGCCCNNNNCTTCAGCTTCCCGATATCCGACGGTAGTGTNNNNTGTACTCCAGCCTGGACGAGTG |
| MSJmono45 | SEQ ID NO: 59 | TTCCTGGCGTGTCCTGTGCTGCTCCAGCNNNNCTTCAGCTTCCCGATATCCGACGGTAGTGTNNNNGCCTGGTGACATAGGAA |
| MSJmono46 | SEQ ID NO: 60 | GATAAATGGAGCTTCAGGTATGGCATGNNNNCTTCAGCTTCCCGATATCCGACGGTAGTGTNNNNACATAAATCTAAAACCA |

TABLE G
Additional microsatellite markers that may be used in combination with the markers of the present disclosure

| | | Microsatellite | Microsatellite | Microsatellite | Microsatellite | | SNP |
|---|---|---|---|---|---|---|---|
| Marker | Chromosome | Base | Length | Start | End | SNP | Position |
| GM07 | chr7 | A | 11 | 93085748 | 93085758 | rs2283006 | 93085722 |
| LR36 | chr4 | A | 12 | 98999723 | 98999734 | rs17550217 | 98999699 |
| LR44 | chr10 | A | 12 | 99898286 | 99898297 | rs7905384 or | 99898268 or |
| | | | | | | rs7905388 | 99898281 |
| LR48 | chr12 | A | 11 | 77988097 | 77988107 | rs11105832 | 77988123 |
| LR52 | chr16 | A | 12 | 63861441 | 63861452 | rs2434849 | 63861437 |

TABLE H
32 Microsatellite Instability Markers

| | | Microsatellite | Microsatellite | Microsatellite | Microsatellite | | SNP |
|---|---|---|---|---|---|---|---|
| Marker | Chromosome | length | Base | Start | End | SNP | Position |
| AKMmono01v2 | 1 | 12 | T | 163944658 | 163944669 | rs12034420 | 163944712 |
| AKMmono02 | 1 | 12 | T | 231388450 | 231388461 | NA | NA |
| AKMmono03 | 2 | 13 | A | 20514738 | 20514750 | NA | NA |
| AKMmono05 | 3 | 13 | A | 47153240 | 47153252 | NA | NA |
| AKMmono08v2 | 6 | 11 | T | 119660766 | 119660776 | rs195082 | 119660876 |
| AKMmono10v2 | 7 | 11 | T | 8110837 | 8110847 | rs10486207 | 8110780 |
| AKMmono11 | 1 | 12 | A | 188783747 | 188783758 | NA | NA |
| AKMmono12 | 1 | 12 | A | 220008901 | 220008912 | NA | NA |
| AKMmono13 | 2 | 12 | A | 95829922 | 95829933 | NA | NA |
| AKMmono16 | 2 | 12 | A | 146400083 | 146400094 | NA | NA |
| AKMmono17v2 | 8 | 11 | A | 59843890 | 59843900 | rs7834158 | 59843813 |
| AKMmono22 | 9 | 14 | A | 104829958 | 104829971 | NA | NA |
| EJmono05 | 9 | 11 | A | 28483545 | 28483555 | NA | NA |
| EJmono13v2 | 5 | 11 | T | 4294954 | 4294964 | rs16873198 | 4295050 |
| EJmono14v2 | 5 | 11 | T | 103200293 | 103200303 | rs2562279 | 103200197 |
| EJmono16 | 10 | 13 | A | 57222859 | 57222871 | NA | NA |
| EJmono21 | 18 | 11 | A | 7068797 | 7068807 | rs7234998 | 7068724 |
| LMmono01 | 14 | 11 | A | 89137211 | 89137221 | NA | NA |
| LMmono03 | 17 | 14 | T | 58399233 | 58399246 | NA | NA |
| LMmono04v2 | 18 | 13 | A | 53192468 | 53192480 | rs10401120 | 53192498 |
| LMmono05v2 | 20 | 13 | A | 33745137 | 33745149 | rs6088734 | 33745046 |
| LMmono09 | 19 | 14 | A | 49540384 | 49540397 | NA | NA |
| LMmono10v2 | 20 | 13 | T | 13814245 | 13814257 | rs10485769 | 13814346 |
| LMmono12 | 16 | 13 | T | 80521284 | 80521296 | NA | NA |
| LMmono16 | 1 | 15 | A | 150737578 | 150737592 | NA | NA |
| MSJmono10 | 1 | 12 | A | 219386785 | 219386796 | NA | NA |
| MSJmono19ms1 | 12 | 14 | A | 80004543 | 80004556 | NA | NA |
| MSJmono19ms2 | 12 | 11 | A | 80004578 | 80004588 | NA | NA |
| MSJmono30v2 | 4 | 13 | T | 140348882 | 140348894 | rs13136124 | 140348815 |
| MSJmono36 | 15 | 13 | A | 56017792 | 56017804 | NA | NA |
| MSJmono44 | 4 | 11 | T | 1911366 | 1911376 | NA | NA |
| MSJmono46 | 19 | 13 | A | 38154539 | 38154551 | NA | NA |

TABLE I
Markers and primers selected for single-round multiplex PCR

| Set | MSI Marker | Primer Name | Primer Sequence (5′ to 3′) |
|---|---|---|---|
| New | AKMmono02_ | AKMmono02_SR_SIxxxF | CAAGCAGAAGACGGCATACGAGAT[Index8N]ACACGCACGATCCGACGGTAGTGTAACTCCTCTAAGGAATATGCTCTC |
| | SNP1 | | (SEQ ID NO: 61) |
| | | AKMmono02_SR_UR | AATGATACGGCGACCACCGAGATCTACACATACGAGATCCGTAATCGGGAAGCTGAAGACACACTAGTTGCATACATGC |
| | | | TT (SEQ ID NO: 62) |
| New | AKMmono03_ | AKMmono03_SR_SIxxxF | CAAGCAGAAGACGGCATACGAGAT[Index8N]ACACGCACGATCCGACGGTAGTGTGGCATTCAAGTCTAACAGAGCTT |
| | SNP1 | | A (SEQ ID NO: 63) |
| | | AKMmono03_SR_UR | AATGATACGGCGACCACCGAGATCTACACATACGAGATCCGTAATCGGGAAGCTGAAGTGATGCCCATCTTTCGTACTT |
| | | | (SEQ ID NO: 64) |
| New | AKMmono04_ | AKMmono04_SR_SIxxxF | CAAGCAGAAGACGGCATACGAGAT[Index8N]ACACGCACGATCCGACGGTAGTGTTGGACACTGACCAAATTGTCAAA |
| | SNP1 | | C (SEQ ID NO: 65) |
| | | AKMmono04_SR_UR | AATGATACGGCGACCACCGAGATCTACACATACGAGATCCGTAATCGGGAAGCTGAAGAAGCAATTAAACACACTTTTG |
| | | | GTG (SEQ ID NO: 66) |
| New | AKMmono07_ | AKMmono07_SR_SIxxxF | CAAGCAGAAGACGGCATACGAGAT[Index8N]ACACGCACGATCCGACGGTAGTGTAAGGCCTTTTCCCTAAGGTG |
| | SNP1 | | |
| | | AKMmono07_SR_UR | AATGATACGGCGACCACCGAGATCTACACATACGAGATCCGTAATCGGGAAGCTGAAGTGGGGCACTTAGATTTTACAG |
| | | | C (SEQ ID NO: 66) |
| New | AKMmono12_ | AKMmono12_SR_SIxxxF | CAAGCAGAAGACGGCATACGAGAT[Index8N]ACACGCACGATCCGACGGTAGTGTCCACTACTGTTTATGCAGCAAG |
| | SNP1 | | |
| | | AKMmono12_SR_UR | AATGATACGGCGACCACCGAGATCTACACATACGAGATCCGTAATCGGGAAGCTGAAGGCTGGAGTTTTCAAATTACAC |
| | | | TG (SEQ ID NO: 67) |
| New | AKMmono13_ | AKMmono13_SR_SIxxxF | CAAGCAGAAGACGGCATACGAGAT[Index8N]ACACGCACGATCCGACGGTAGTGTAATAAAAACCACCACACAGAAGG |
| | SNP1 | | (SEQ ID NO: 68) |
| | | AKMmono13_SR_UR | AATGATACGGCGACCACCGAGATCTACACATACGAGATCCGTAATCGGGAAGCTGAAGGAGCTTGATTAAAGTCACAAC |
| | | | ATC (SEQ ID NO: 69) |
| New | AKMmono16_ | AKMmono 16_SR_SIxxxF | CAAGCAGAAGACGGCATACGAGAT[Index8N]ACACGCACGATCCGACGGTAGTGTATGCATTTGCTCCTTCACACTTG |
| | SNP1 | | (SEQ ID NO: 70) |
| | | AKMmono 16_SR_UR | AATGATACGGCGACCACCGAGATCTACACATACGAGATCCGTAATCGGGAAGCTGAAGCACTGAGGCAGTATCTCTGTC |
| | | | AT (SEQ ID NO: 71) |
| New | EJmono12_ | EJmono12_SR_SIxxxF | CAAGCAGAAGACGGCATACGAGAT[Index8N]ACACGCACGATCCGACGGTAGTGTACTTGAGAAAGATGTGGTCAACT |
| | SNP1 | | (SEQ ID NO: 72) |
| | | EJmono12_SR_UR | AATGATACGGCGACCACCGAGATCTACACATACGAGATCCGTAATCGGGAAGCTGAAGACAGGTTTACAAATTCCCACC |
| | | | T (SEQ ID NO: 73) |
| New | MSJmono20_ | MSJmono20_SR_SIxxxF | CAAGCAGAAGACGGCATACGAGAT[Index8N]ACACGCACGATCCGACGGTAGTGTATTGTACTCCAGCACGGGT |
| | SNP1 | MSJmono20_SR_UR | AATGATACGGCGACCACCGAGATCTACACATACGAGATCCGTAATCGGGAAGCTGAAGATTTTACTTCCTCATTTTCCTG |
| | | | GAG (SEQ ID NO: 74) |
| New | MSJmono39_ | MSJmono39_SR_SIxxxF | CAAGCAGAAGACGGCATACGAGAT[Index8N]ACACGCACGATCCGACGGTAGTGTACTTAGCTAGATAAAACACACATT |
| | SNP1 | | T (SEQ ID NO: 75) |
| | | MSJmono39_SR_UR | AATGATACGGCGACCACCGAGATCTACACATACGAGATCCGTAATCGGGAAGCTGAAGCCATGTATCGTATTTACTGGT |
| | | | GGT (SEQ ID NO: 76) |
| New | MSJmono45_ | MSJmono45_SR_SIxxxF | CAAGCAGAAGACGGCATACGAGAT[Index8N]ACACGCACGATCCGACGGTAGTGTTCATGCTACTGCACTCCAGC (SEQ |
| | SNP1 | | ID NO: 77) |
| | | MSJmono45_SR_UR | AATGATACGGCGACCACCGAGATCTACACATACGAGATCCGTAATCGGGAAGCTGAAGGGAACAGACGGGCCAAACT |
| | | | (SEQ ID NO: 78) |
| Original | GM07_SNP1 | GM07_SR_SIxxxF | CAAGCAGAAGACGGCATACGAGAT[Index8N]ACACGCACGATCCGACGGTAGTGTGGGCCCTTTTAGGCATATAG (SEQ |
| | | | ID NO: 79) |
| | | GM07_SR_UR | AATGATACGGCGACCACCGAGATCTACACATACGAGATCCGTAATCGGGAAGCTGAAGCATATGGGGTTTGGTCACA |
| | | | (SEQ ID NO: 80) |
| Original | LR36_SNP1 | LR36_SR_SIxxxF | CAAGCAGAAGACGGCATACGAGAT[Index8N]ACACGCACGATCCGACGGTAGTGTGCTGGCACTTGTGGTGAC (SEQ |
| | | | ID NO: 81) |
| | | LR36_SR_UR | AATGATACGGCGACCACCGAGATCTACACATACGAGATCCGTAATCGGGAAGCTGAAGTTTGCACTCTTTGCACACC |
| | | | (SEQ ID NO: 82) |
| Original | LR44_SNP1 | LR44_SR_SIxxxF | CAAGCAGAAGACGGCATACGAGAT[Index8N]ACACGCACGATCCGACGGTAGTGTAGGCTCATTTGAGGCCAAG (SEQ |
| | | | ID NO: 83) |
| | | LR44_SR_UR | AATGATACGGCGACCACCGAGATCTACACATACGAGATCCGTAATCGGGAAGCTGAAGAAAAACAGTCAAGGAACAAA |
| | | | AGTG (SEQ ID NO: 84) |
| | | KRAS_Q61_SR_UR2 | AATGATACGGCGACCACCGAGATCTACACATACGAGATCCGTAATCGGGAAGCTGAAGACCTGTCTCTTGGATATTCTCG |
| | | | AC (SEQ ID NO: 90) |

### EXAMPLES

### Example 1

In the context of the present description, including the Examples provided below, when reference is made to original marker or original MSI assay, this refers to the markers and/or assay as described in WO2021019197.

1. Identification of Candidate Microsatellite Markers from Whole Genome Sequence Data

### 1.1. Identification of Microsatellite Loci Using Whole Genome Sequencing

### Samples:

3 CMMRD peripheral blood leukocyte genomic DNAs; 1 LS peripheral blood leukocyte genomic DNA and 2 control peripheral blood leukocyte genomic DNAs were used.

### Method and Analysis:

PCR amplification of samples using NEBNext® Ultra™ II DNA Library Prep Kit for Illumina (New England Biolabs) was performed, followed by high depth (120×) genome sequencing on a NovaSeq (Illumina). A custom bioinformatics pipeline was then used to detect microsatellite variants in the genome sequence and microsatellite loci with variant allele frequencies suggestive of somatic instability in the CMMRD and LS samples, but not in the control samples were selected.

### Results:

191 microsatellite loci, including mono-, di-, tri-, tetra- and pentanucleotide repeats, as well as complex microsatellites containing multiple motifs, were identified from the whole genome sequence data.

1.2. Design and Confirmation of smMIPs to Capture Candidate Microsatellite Markers

### Samples:

3 FFPE tumour genomic DNAs and 3 control peripheral blood leukocyte genomic DNAs were used.

### Method and Analysis:

smMIPs to capture the identified microsatellite loci were designed using MIPgen software (Boyle et al. Bioinformatics. 2014 Sep. 15; 30(18):2670-2, DOI: 10.1093/bioinformatics/btu353. PMID: 24867941). smMIP-based amplification of microsatellite loci from samples was then performed, followed by high depth (1000×) amplicon sequencing on a MiSeq (Illumina). Finally, read depths achieved for each smMIP as a check of smMIP performance was calculated.

### Results:

MIPgen failed to design a smMIP for 21 of the 191 microsatellite loci. Therefore, 170 microsatellite loci had a smMIP available to be checked by amplicon sequencing. Some loci contained multiple distinct microsatellites and, therefore, these 170 smMIPs capture a total of 213 candidate microsatellite markers.

smMIPs were analysed in batches and smMIPs generating read counts >10% of the median read depth passed the smMIP quality check, totalling 133 of the 170 smMIPs. These 133 smMIPs capture 155 candidate microsatellite markers, including mono-, di-, tri-, tetra- and pentanucleotide repeats, as well as complex microsatellites containing multiple motifs.

Mononucleotide repeat markers are of particular interest as they are sensitive to deficiency of any MMR protein, whereas longer motif microsatellite markers (such as dinucleotide repeats) are not sensitive to MSH6 deficiency. 91 of the 133 smMIPs that passed the quality check capture at least one mononucleotide repeat and, in total, capture 98 mononucleotide repeats between them.

All 91 smMIPs and the candidate 98 mononucleotide repeat markers they capture were taken forward for additional analysis using smMIP-based amplification and amplicon sequencing of a pilot cohort of samples.

### 2. Candidate Marker Selection Using a Pilot Cohort of Blood and Tumour DNA Samples

### Samples:

8 CMMRD peripheral blood leukocyte genomic DNAs; 38 control peripheral blood leukocyte genomic DNAs; 8 MMR deficient CRC genomic DNAs and 8 MMR proficient CRC genomic DNAs were used.

### Method and Analysis:

smMIP-based amplification of the candidate 98 mononucleotide repeat markers from samples, followed by high depth (5000×) amplicon sequencing on a MiSeq (Illumina) was performed.

A custom bioinformatics pipeline to extract microsatellite allele frequencies from the amplicon sequence data was used. Estimation of the frequency of germline length variants in each candidate marker using the blood samples was performed. Finally, assessment of microsatellite allele distribution by visual inspection of graphs of microsatellite allele frequencies was carried out. Many aspects of the distributions were considered to determine if the marker had unambiguous signals of increased MSI in the MMR deficient tumour or blood samples. Markers were then given assigned to groups, where group 1 contained markers with the clearest MSI signal, down to group 4 which contained markers with no clear MSI signal.

Assessment of microsatellite reference allele frequency (RAF) as a measure of MSI in different sample types, and generation of receiver operator characteristic area under curve (ROC AUC) statistics based on RAF as a measure of marker sensitivity and specificity for MMR deficiency was performed. Analyses of tumour DNA samples used read frequencies, whereas analyses of blood DNA samples used smSequence frequencies (“read” and “smSequence” are defined as per Gallon et al. 2019).

Candidate mononucleotide repeat markers for inclusion in a version 2 MSI assay to be assessed in larger sample cohorts were selected. For a version 2 tumour MSI assay we aimed for approximately 50 markers, and for a version 2 CMMRD MSI assay (described herein) we aimed for approximately 30 markers (MSI analysis to detect CMMRD requires much higher read depths, so fewer markers were used to reduce the total reads needed and, therefore, the cost of sequencing). In the context of the present disclosure, the term “version 2 MSI assay” refers to the assays and/or MSI markers that are described herein.

### Results:

All samples, except 7 control samples, had previously been analysed by the original 24 mononucleotide markers using the same smMIP-based method (disclosed in WO2021019197), allowing comparison of the new candidate mononucleotide repeat markers and original mononucleotide repeat markers. For these pilot comparisons, it should be noted that the original markers have already been through several rounds of selection to optimise the panel for the detection of MMR deficiency in CRCs and so also contain good markers for the detection of CMMRD in non-neoplastic tissues.

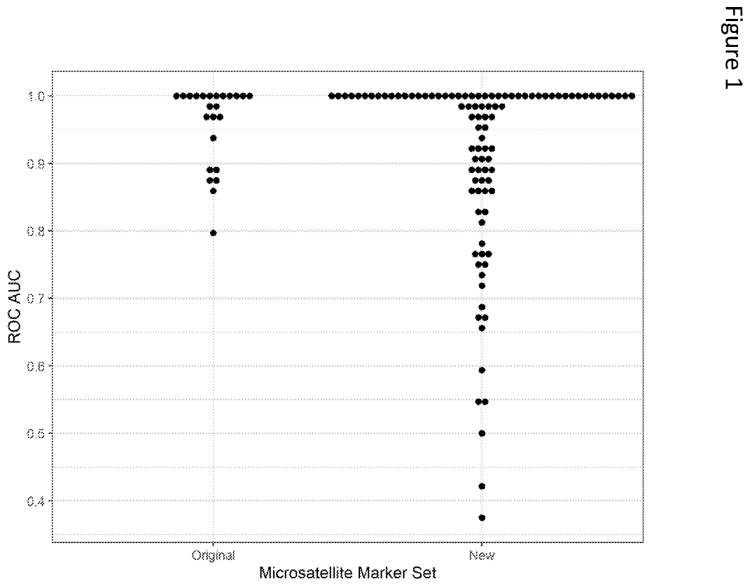

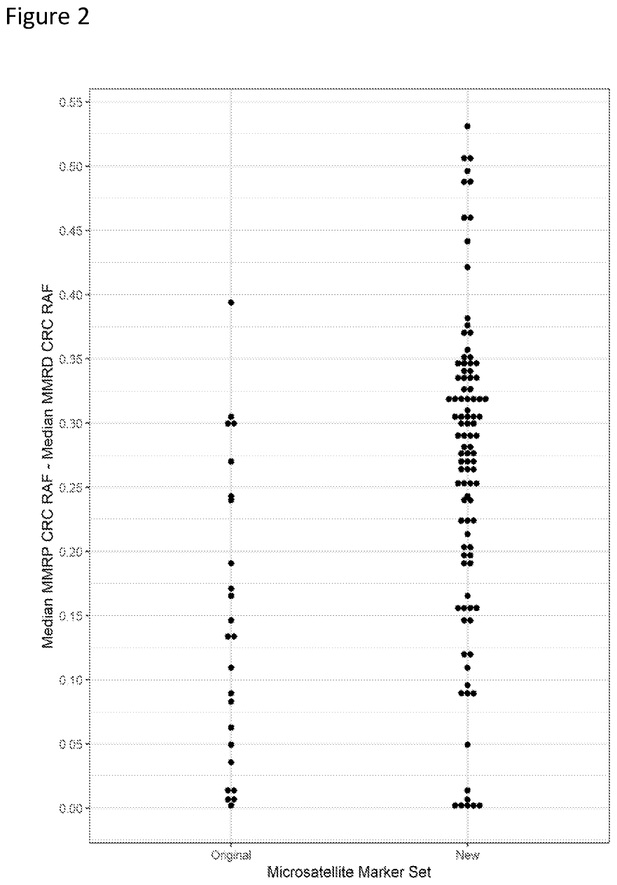

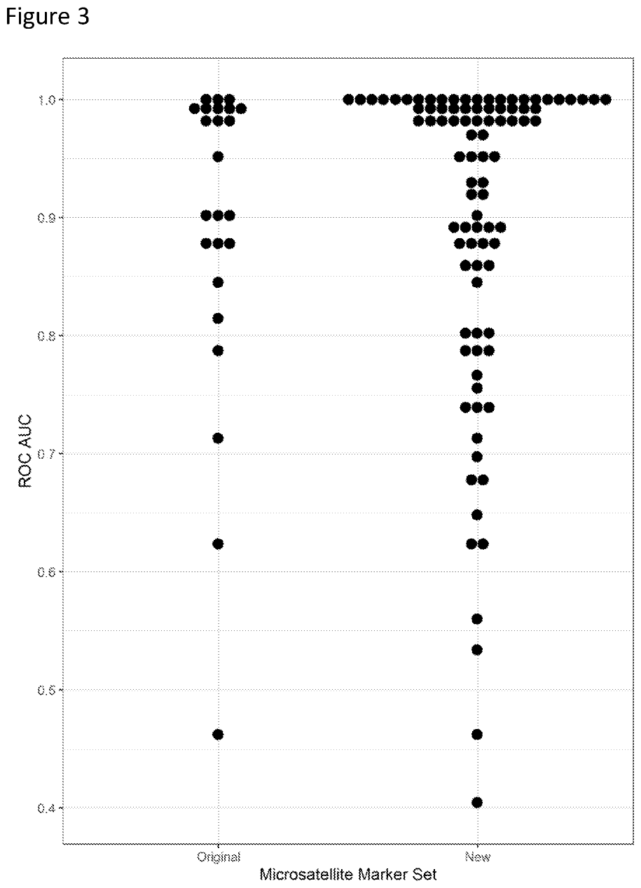

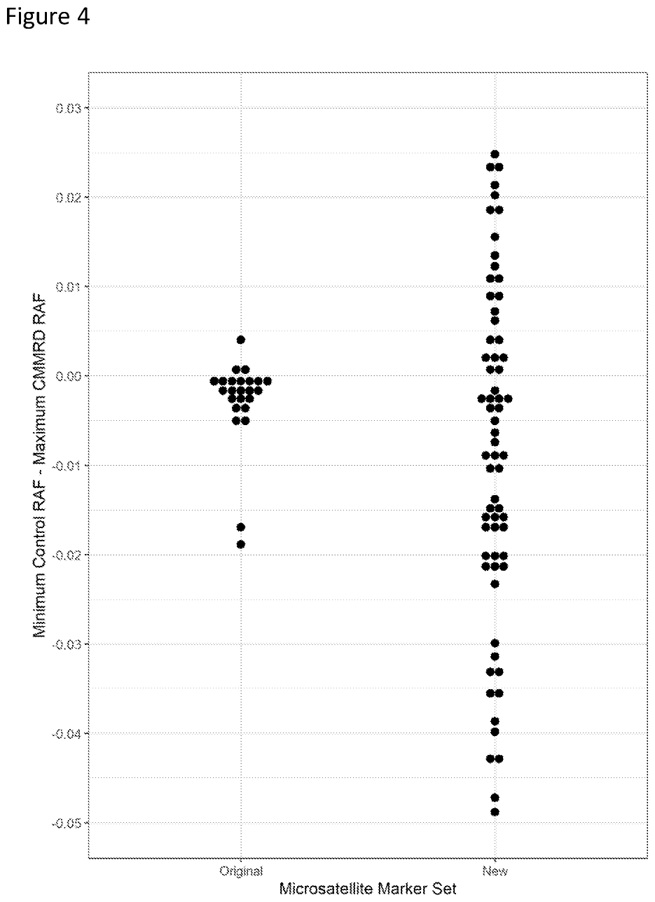

Many of the new candidate mononucleotide repeat markers were equivalent or superior to the original mononucleotide repeat markers for the detection of MMR deficiency in different sample types. For example:
using RAF as a measure of MSI to detect MMR deficiency in CRCs, 59/98 (60.2%) of the new candidate markers and 17/24 (70.8%) of the original markers had a ROC AUC >0.95 (FIG. 1).
The difference in the median RAF of the MMR proficient versus MMR deficient CRCs (median difference) was generally greater among the new candidate markers versus the original markers (FIG. 2, Mann Whitney U Test p=6.5×10⁻⁵).
using RAF as a measure of MSI to detect CMMRD, 49/98 (50.0%) of the new candidate markers and 12/24 (50.0%) of the original markers had a ROC AUC >0.95 (FIG. 3).
the difference in the minimum RAF of the controls and the maximum RAF of the CMMRDs (margin difference) ranged up to 0.025 among the new candidate markers, whereas the greatest margin difference observed with the original markers was 0.004 (FIG. 4).

The 62 best of the new candidate mononucleotide repeat markers (captured by 60 smMIPs) were selected from the smMIP amplicon sequence data, based on microsatellite allele distribution, RAF ROC AUC for the detection of MMR deficiency in both tumour and blood samples, and frequency of germline length variants (Table 1). All 62 mononucleotide repeat markers were taken forward for additional analysis using smMIP-based amplification and amplicon sequencing of a large colorectal cancer cohorts.

These 62 mononucleotide repeat markers were further ranked by RAF ROC AUC and margin difference for the detection of CMMRD versus control samples. The best 32 (captured by 31 smMIPs) were selected (Table 1) for further assessment using smMIP-based amplification and amplicon sequencing of a large blinded cohort of CMMRD and control sample.

TABLE 1
Selection of candidate mononucleotide repeat markers to create version 2 MSI assays. Selection criteria
for version 2 tumour MSI assay included: Germline variant frequency <0.10, ROC AUC >0.90 for detection
of MMR deficiency in either blood or tumour samples, and placement in allele distribution group 1 or
2 (see Methods and Analysis). Further selection criteria for the version 2 CMMRD assay included: Germline
variant frequency <0.05, ROC AUC >0.95 for detection of MMR deficiency in blood samples, minimum
control blood RAF >0.88, and margin difference (minimum control RAF − maximum CMMRD RAF) > −0.02.

| | | | | | Tumour | Blood | Min. | Min. Control |
|---|---|---|---|---|---|---|---|---|
| Candidate | Select for | Select for | Germline | Allele | RAF | RAF | Control | Blood RAF − |
| Mononucleotide | Tumour MSI | CMMRD MSI | Variant | Distribution | ROC | ROC | Blood | Max. CMMRD |
| Repeat Markers | Assay | Assay | Frequency | Group | AUC | AUC | RAF | RAF |
| AKMmono02 | TRUE | TRUE | 0.00 | 1 | 1.00 | 0.98 | 0.96 | −0.01 |
| AKMmono03 | TRUE | TRUE | 0.00 | 1 | 1.00 | 0.95 | 0.95 | −0.02 |
| AKMmono05 | TRUE | TRUE | 0.00 | 1 | 1.00 | 0.98 | 0.95 | −0.01 |
| AKMmono06 | TRUE | FALSE | 0.00 | 1 | 1.00 | 0.99 | 0.86 | −0.02 |
| AKMmono07 | TRUE | FALSE | 0.00 | 1 | 1.00 | 0.93 | 0.77 | −0.14 |
| AKMmono08 | TRUE | TRUE | 0.00 | 1 | 1.00 | 1.00 | 0.98 | 0.00 |
| AKMmono10 | TRUE | TRUE | 0.00 | 1 | 1.00 | 1.00 | 0.98 | 0.02 |
| AKMmono11 | TRUE | TRUE | 0.04 | 1 | 1.00 | 1.00 | 0.97 | 0.01 |
| AKMmono12 | TRUE | TRUE | 0.00 | 1 | 1.00 | 0.95 | 0.95 | −0.01 |
| AKMmono13 | TRUE | TRUE | 0.00 | 1 | 1.00 | 1.00 | 0.99 | 0.02 |
| AKMmono14 | TRUE | FALSE | 0.00 | 1 | 1.00 | 0.88 | 0.96 | −0.04 |
| AKMmono16 | TRUE | TRUE | 0.00 | 1 | 1.00 | 1.00 | 0.97 | 0.01 |
| AKMmono17 | TRUE | TRUE | 0.00 | 1 | 1.00 | 0.99 | 0.98 | 0.00 |
| EJmono01 | TRUE | FALSE | 0.02 | 1 | 1.00 | 0.53 | 0.81 | −0.17 |
| EJmono06 | TRUE | FALSE | 0.00 | 1 | 1.00 | 0.98 | 0.75 | −0.04 |
| EJmono12 | TRUE | FALSE | 0.02 | 1 | 1.00 | 0.95 | 0.79 | −0.13 |
| EJmono14 | TRUE | TRUE | 0.00 | 1 | 1.00 | 0.99 | 0.96 | −0.01 |
| EJmono21 | TRUE | TRUE | 0.04 | 1 | 1.00 | 1.00 | 0.98 | 0.00 |
| LMmono04 | TRUE | TRUE | 0.02 | 1 | 1.00 | 1.00 | 0.95 | 0.00 |
| LMmono05 | TRUE | TRUE | 0.02 | 1 | 1.00 | 1.00 | 0.97 | 0.01 |
| LMmono16 | TRUE | TRUE | 0.00 | 1 | 1.00 | 0.97 | 0.89 | −0.02 |
| AKMmono22 | TRUE | TRUE | 0.04 | 1 | 1.00 | 1.00 | 0.92 | −0.01 |
| EJmono03 | TRUE | FALSE | 0.00 | 1 | 1.00 | 0.98 | 0.94 | −0.02 |
| LMmono01 | TRUE | TRUE | 0.00 | 1 | 1.00 | 0.99 | 0.94 | −0.01 |
| LMmono03 | TRUE | TRUE | 0.02 | 1 | 1.00 | 1.00 | 0.92 | 0.02 |
| MSJmono19ms1 | TRUE | TRUE | 0.00 | 1 | 1.00 | 1.00 | 0.98 | 0.00 |
| MSJmono19ms2 | TRUE | TRUE | 0.02 | 1 | 1.00 | 0.89 | 0.98 | −0.02 |
| MSJmono20 | TRUE | FALSE | 0.00 | 1 | 1.00 | 0.89 | 0.92 | −0.04 |
| MSJmono22 | TRUE | FALSE | 0.00 | 1 | 1.00 | 0.81 | 0.95 | −0.05 |
| MSJmono23 | TRUE | FALSE | 0.02 | 1 | 1.00 | 0.97 | 0.95 | −0.02 |
| MSJmono26 | TRUE | FALSE | 0.00 | 1 | 1.00 | 0.88 | 0.83 | −0.10 |
| MSJmono27 | TRUE | FALSE | 0.02 | 1 | 1.00 | 0.68 | 0.83 | −0.13 |
| MSJmono37 | TRUE | FALSE | 0.02 | 1 | 1.00 | 0.87 | 0.95 | −0.03 |
| MSJmono38 | TRUE | FALSE | 0.00 | 1 | 1.00 | 0.89 | 0.97 | −0.02 |
| MSJmono39 | TRUE | FALSE | 0.00 | 1 | 1.00 | 0.84 | 0.98 | −0.02 |
| MSJmono41 | TRUE | FALSE | 0.00 | 1 | 1.00 | 0.78 | 0.96 | −0.02 |
| MSJmono44 | TRUE | TRUE | 0.00 | 1 | 1.00 | 1.00 | 0.98 | 0.02 |
| MSJmono46 | TRUE | TRUE | 0.00 | 1 | 1.00 | 0.99 | 0.92 | 0.00 |
| EJmono05 | TRUE | TRUE | 0.00 | 1 | 0.98 | 0.99 | 0.96 | 0.00 |
| EJmono16 | TRUE | TRUE | 0.02 | 1 | 0.98 | 0.98 | 0.96 | −0.01 |
| LMmono08 | TRUE | FALSE | 0.00 | 1 | 0.98 | 0.46 | 0.90 | −0.10 |
| LMmono10 | TRUE | TRUE | 0.00 | 1 | 0.98 | 1.00 | 0.92 | 0.02 |
| LMmono12 | TRUE | TRUE | 0.00 | 1 | 0.98 | 1.00 | 0.92 | 0.01 |
| MSJmono10 | TRUE | TRUE | 0.02 | 1 | 0.98 | 1.00 | 0.98 | 0.00 |
| MSJmono30 | TRUE | TRUE | 0.00 | 1 | 0.97 | 0.99 | 0.96 | −0.01 |
| MSJmono40 | TRUE | FALSE | 0.00 | 1 | 0.97 | 0.74 | 0.94 | −0.05 |
| LMmono09 | TRUE | TRUE | 0.00 | 1 | 0.95 | 1.00 | 0.88 | 0.01 |
| MSJmono15 | TRUE | FALSE | 0.00 | 1 | 0.94 | 0.88 | 0.98 | −0.02 |
| LMmono07 | TRUE | FALSE | 0.07 | 1 | 0.92 | 1.00 | 0.98 | 0.00 |
| MSJmono11 | TRUE | FALSE | 0.00 | 1 | 0.91 | 0.90 | 0.89 | −0.04 |
| EJmono02 | TRUE | FALSE | 0.04 | 1 | 0.86 | 0.98 | 0.94 | −0.03 |
| MSJmono36 | TRUE | TRUE | 0.02 | 1 | 0.86 | 1.00 | 0.97 | 0.00 |
| AKMmono01 | TRUE | TRUE | 0.02 | 1 | 0.78 | 1.00 | 0.98 | 0.01 |
| EJmono13 | TRUE | TRUE | 0.04 | 1 | 0.75 | 0.99 | 0.98 | 0.00 |
| AKMmono04 | TRUE | FALSE | 0.04 | 2 | 1.00 | 0.86 | 0.94 | −0.04 |
| HGtetra23ms2 | TRUE | FALSE | 0.02 | 2 | 1.00 | 0.92 | 0.96 | −0.02 |
| MSJcom06ms2 | TRUE | FALSE | 0.00 | 2 | 1.00 | 0.86 | 0.66 | −0.34 |
| MSJmono45 | TRUE | FALSE | 0.04 | 2 | 1.00 | 0.79 | 0.84 | −0.16 |
| MSJcom06ms1 | TRUE | FALSE | 0.02 | 2 | 0.98 | 0.80 | 0.67 | −0.31 |
| EJmono04 | TRUE | FALSE | 0.04 | 2 | 0.92 | 0.98 | 0.87 | −0.02 |
| MSJmono32_SNP1 | TRUE | FALSE | 0.04 | 2 | 0.91 | 0.92 | 0.67 | −0.27 |
| MSJmono17 | TRUE | FALSE | 0.02 | 2 | 0.88 | 0.95 | 0.87 | −0.10 |
| MSJmono33 | FALSE | FALSE | 0.00 | 3 | 0.89 | 0.78 | 0.73 | −0.20 |
| MSJmono01ms2 | FALSE | FALSE | 0.02 | 3 | 0.38 | 0.62 | 0.98 | −0.02 |
| MSJmono29_SNP1 | FALSE | FALSE | 0.07 | 3 | 1.00 | 1.00 | 0.97 | 0.01 |
| AKMmono21ms2 | FALSE | FALSE | 0.02 | 3 | 0.95 | 0.86 | 0.77 | −0.23 |
| LMmono06 | FALSE | FALSE | 0.02 | 3 | 0.89 | 0.67 | 0.74 | −0.15 |
| LMmono02 | FALSE | FALSE | 0.11 | 3 | 1.00 | 1.00 | 0.98 | 0.02 |
| MSJmono34ms1 | FALSE | FALSE | 0.30 | 3 | 0.97 | 0.76 | 0.66 | −0.34 |
| MSJmono24 | FALSE | FALSE | 0.13 | 3 | 0.91 | 1.00 | 0.96 | 0.00 |
| MSJmono35 | FALSE | FALSE | 0.00 | 3 | 0.89 | 0.70 | 0.65 | −0.33 |
| AKMmono18 | FALSE | FALSE | 0.15 | 3 | 0.88 | 0.99 | 0.97 | 0.00 |
| AKMmono15 | FALSE | FALSE | 0.11 | 3 | 0.86 | 1.00 | 0.89 | 0.02 |
| EJmono11 | FALSE | FALSE | 0.20 | 3 | 1.00 | 0.93 | 0.93 | −0.04 |
| EJmono10 | FALSE | FALSE | 0.28 | 3 | 0.97 | 1.00 | 0.97 | 0.01 |
| EJmono15 | FALSE | FALSE | 0.13 | 3 | 0.92 | 1.00 | 0.93 | 0.02 |
| EJmono07 | FALSE | FALSE | 0.15 | 3 | 0.88 | 0.98 | 0.90 | −0.03 |
| EJmono22 | FALSE | FALSE | 0.15 | 3 | 0.86 | 0.89 | 0.69 | −0.22 |
| EJmono08 | FALSE | FALSE | 0.20 | 3 | 0.77 | 0.99 | 0.94 | 0.00 |
| LMmono15ms2 | FALSE | FALSE | 0.00 | 4 | 0.73 | 0.65 | 0.83 | −0.17 |
| MSJmono34ms2 | FALSE | FALSE | 0.00 | 4 | 0.72 | 0.40 | 0.99 | −0.01 |
| HGtetra18ms2 | FALSE | FALSE | 0.00 | 4 | 0.67 | 0.62 | 0.98 | −0.02 |
| LMmono13 | FALSE | FALSE | 0.52 | 4 | 0.92 | 0.74 | 0.76 | −0.12 |
| MSJmono01ms1 | FALSE | FALSE | 0.26 | 4 | 0.89 | 0.77 | 0.66 | −0.29 |
| EJmono09 | FALSE | FALSE | 0.46 | 4 | 0.83 | 0.98 | 0.92 | −0.03 |
| MSJmono07 | FALSE | FALSE | 0.52 | 4 | 0.83 | 0.80 | 0.66 | −0.30 |
| HGtetra25ms1 | FALSE | FALSE | 0.83 | 4 | 0.81 | NA | NA | NA |
| MSJcom05ms1 | FALSE | FALSE | 0.59 | 4 | 0.77 | 0.72 | 0.70 | −0.07 |
| MSJcom05ms2 | FALSE | FALSE | 0.50 | 4 | 0.77 | 0.89 | 0.69 | −0.30 |
| MSJcom07ms1 | FALSE | FALSE | 0.74 | 4 | 0.75 | NA | NA | NA |
| MSJcom08ms2 | FALSE | FALSE | 0.57 | 4 | 0.69 | 0.56 | 0.67 | −0.26 |
| MSJmono12_SNP1 | FALSE | FALSE | 0.98 | 4 | 0.67 | NA | NA | NA |
| MSJmono03 | FALSE | FALSE | 0.54 | 4 | 0.66 | NA | NA | NA |
| MSJcom04ms1 | FALSE | FALSE | 0.76 | 4 | 0.59 | NA | NA | NA |
| LMmono15ms1 | FALSE | FALSE | 0.65 | 4 | 0.55 | NA | NA | NA |
| MSJmono08 | FALSE | FALSE | 0.78 | 4 | 0.55 | NA | NA | NA |
| AKMmono21ms1 | FALSE | FALSE | 0.65 | 4 | 0.50 | 0.74 | 0.89 | −0.08 |
| LMmono11 | FALSE | FALSE | 0.83 | 4 | 0.42 | NA | NA | NA |

### 3. Selected Markers Improve CMMRD Detection Using a Large Blinded Cohort

### Samples:

30 CMMRD peripheral blood leukocyte genomic DNAs (blinded); 43 control peripheral blood leukocyte genomic DNAs (blinded); and 30 control peripheral blood leukocyte genomic DNAs (known) were used.

### Method and Analysis:

smMIP-based amplification of 32 mononucleotide repeat markers from samples, followed by high depth (5000×) amplicon sequencing on a MiSeq (Illumina) was performed. A custom bioinformatics pipeline to extract microsatellite allele frequencies from the amplicon sequence data was used. Sample scoring using the method of our original MSI assay to detect CMMRD (Gallon et al. 2019; Perez-Valencia et al. Genet Med. 2020 December; 22(12):2081-2088, DOI:10.1038/s41436-020-0925-z, PMID: 32773772) was carried out. The 30 known controls were used as a reference set for sample scoring. A higher score indicates increased MSI and a higher probability the sample is from an individual with CMMRD.

### Results:

All samples, except 3 CMMRD and 1 control sample from the blinded cohort, had previously been analysed by the original 24 mononucleotide repeat markers (described in Gallon et al. 2019. Further information on these markers may also be found in WO2021019197 and WO2018037231) using the same method, allowing comparison of the new and original marker sets.

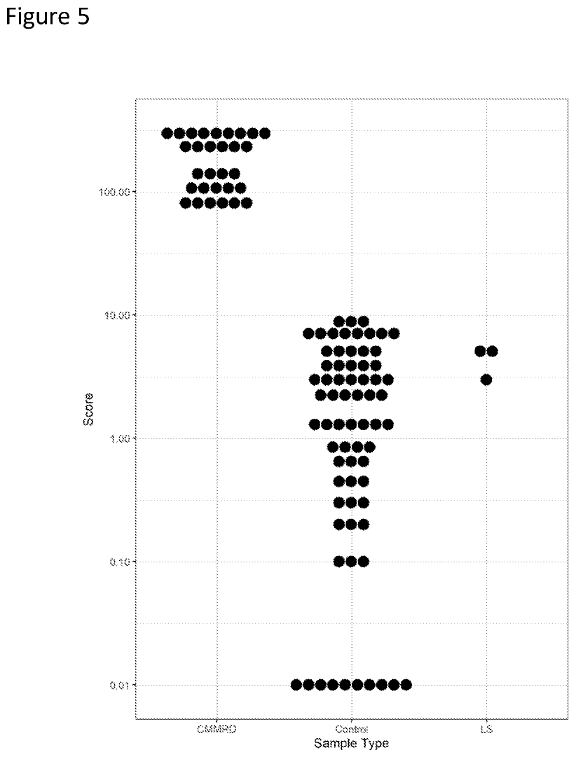

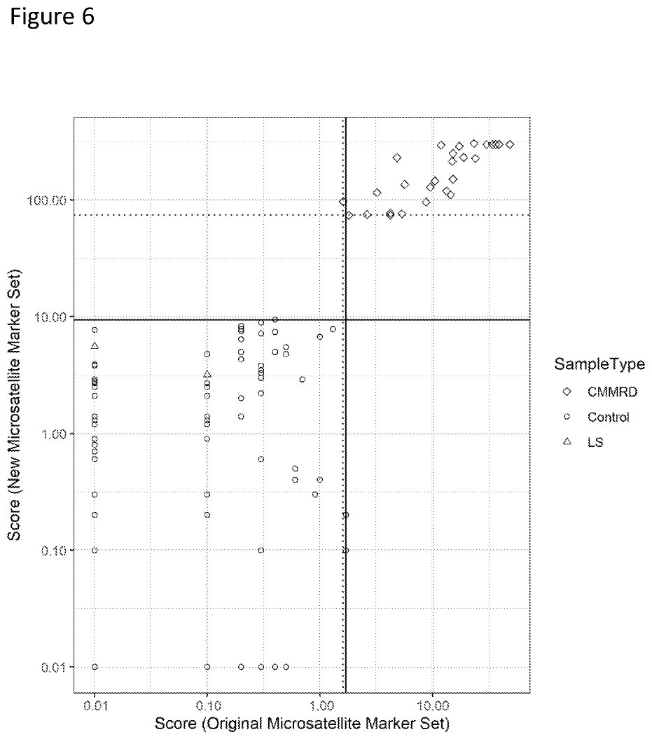

Sample un-blinding showed that the scoring method detected CMMRD samples with 100% sensitivity (95% CI: 88.4-100.0%) and 100% specificity (95% CIs: 95.1-100.0%), with a very large separation (score difference=64.7) between CMMRD and control samples (FIG. 5). This score separation was far greater than that of the original mononucleotide repeat marker set, which had overlapping scores from CMMRD and control samples (FIG. 6). This supports that the process of selection from genome sequence data of individuals with CMMRD has identified exceptional markers for MSI analysis.

Two CMMRD samples (ID210 and ID224) from the blinded cohort had smSequence counts <100 in 18 markers. Whilst the remaining 14 markers clearly showed these two samples to have increased MSI, the samples were excluded from further analyses due to the unreliability of data from them in some markers.

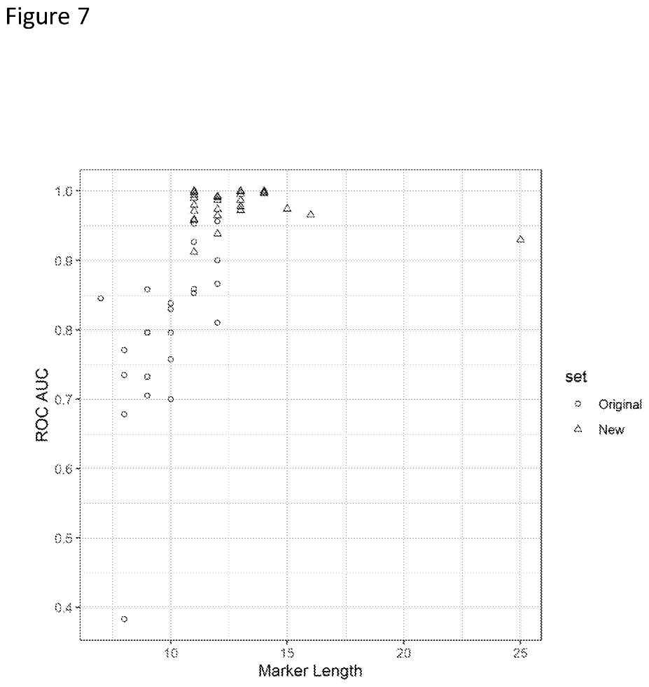

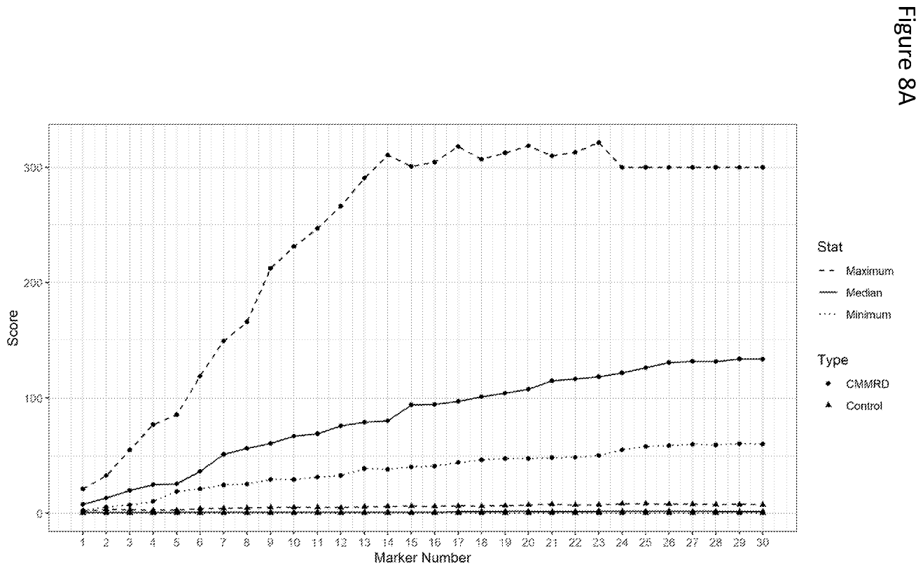

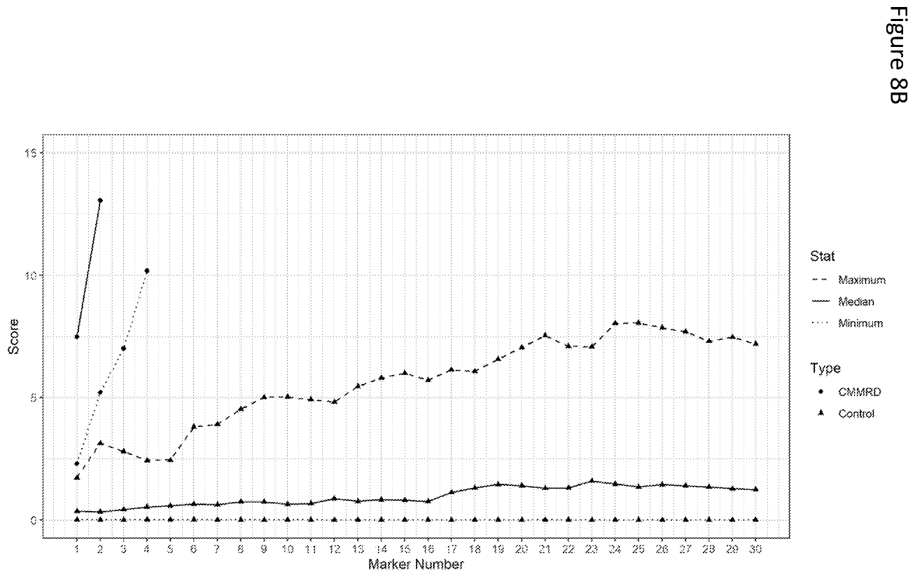

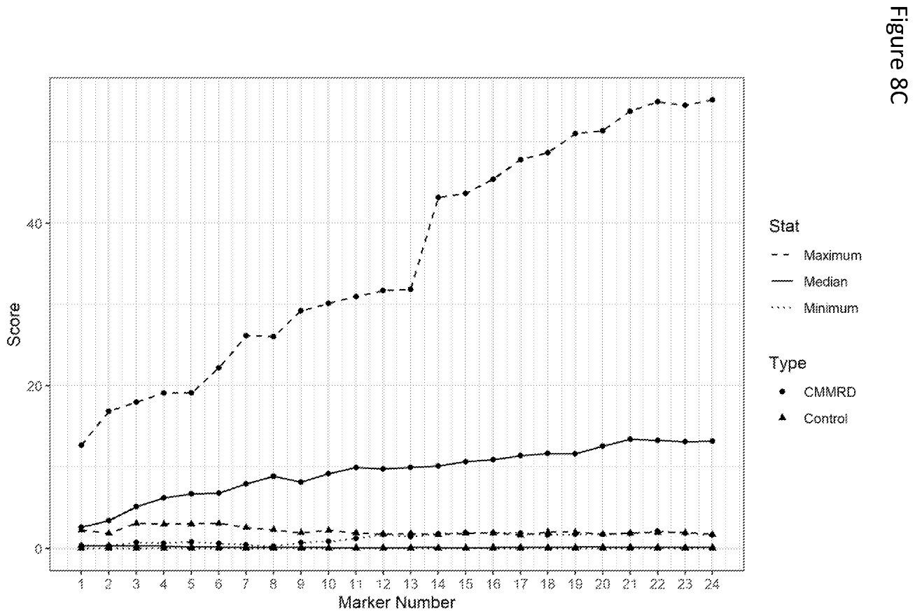

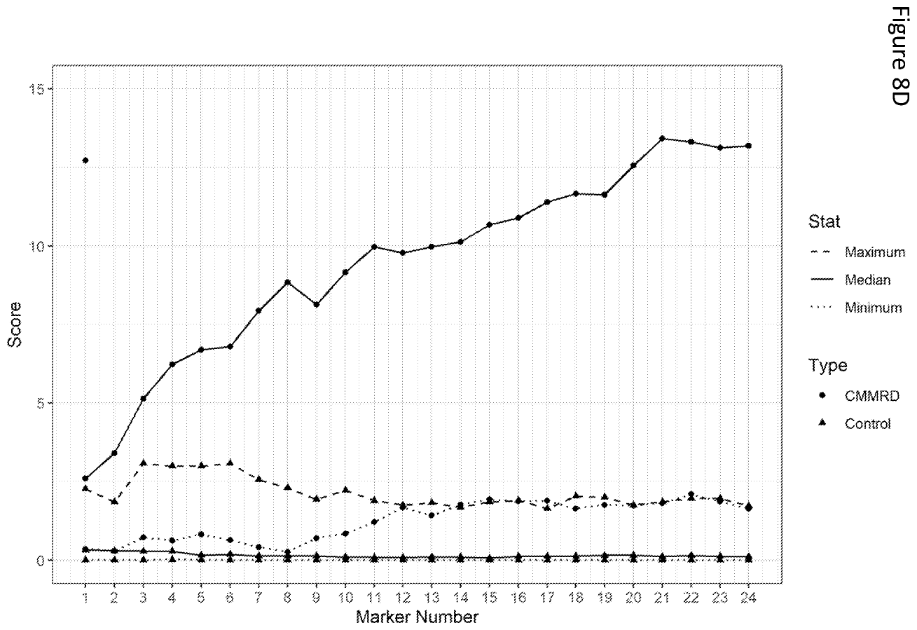

Exploration of microsatellite marker structure across both the original and new mononucleotide repeat markers showed a strong correlation of ROC AUC (using smSequence RAF as measure of MSI to detect CMMRD) and microsatellite length (FIG. 7, Spearman Rho=0.743, p=5.4×10⁻¹¹). New mononucleotide repeat markers were generally longer than the original mononucleotide repeat markers (FIG. 7, Mann Whitney U Test p=2.5×10⁻⁹), suggesting the improved performance of the new markers may be a function of microsatellite structure rather than the selection process. However, a comparison of the new markers of 11 to 12 nucleotides in length with the original markers of the same length showed significantly higher ROC AUCs in the new markers (FIG. 7, Mann Whitney U Test p=5.2×10⁻⁵). This further supports that the process of selection from genome sequence data of individuals with CMMRD has identified exceptional markers for MSI analysis.

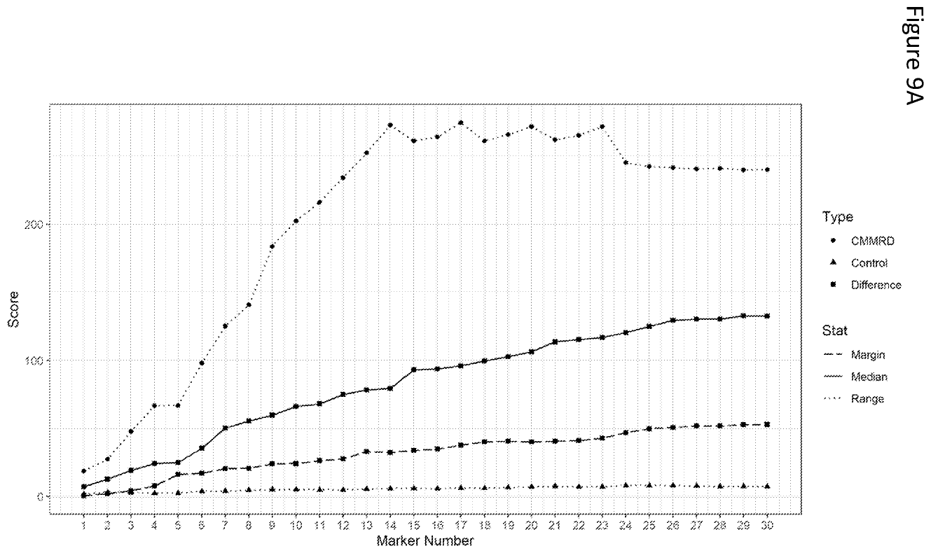

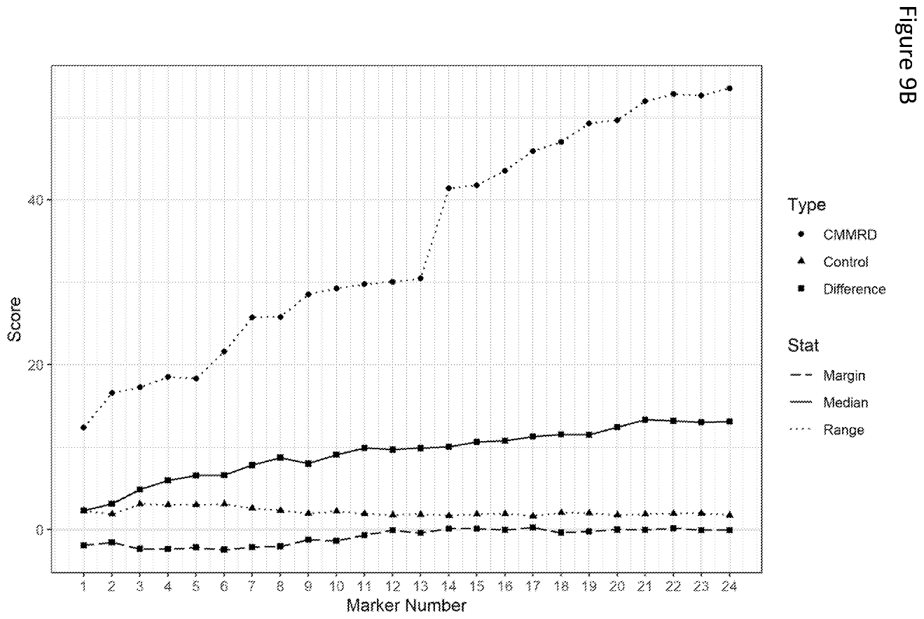

The effect of reducing marker number on assay score distributions was assessed by first ranking all microsatellite markers (from both the new 32 mononucleotide repeats and original 24 mononucleotide repeats) based on their ability to detect CMMRD using smSequence RAF in the blinded cohort, and then repeating scoring using the top n markers, from n=1 through to n=30. Only 2 of the original microsatellite markers were included in the top 30 markers at rank 22 and rank 28 (Table 2). Separation of all CMMRD from all control samples by MSI scoring was achieved with all marker sets, including scoring by the top, single microsatellite marker (FIGS. 8A and 8B). An equivalent analysis that included only the original mononucleotide repeat markers showed persistent overlap of CMMRD and control sample scores (FIGS. 8C and 8D), again supporting that the method of selection has identified exceptional markers for MSI analysis.

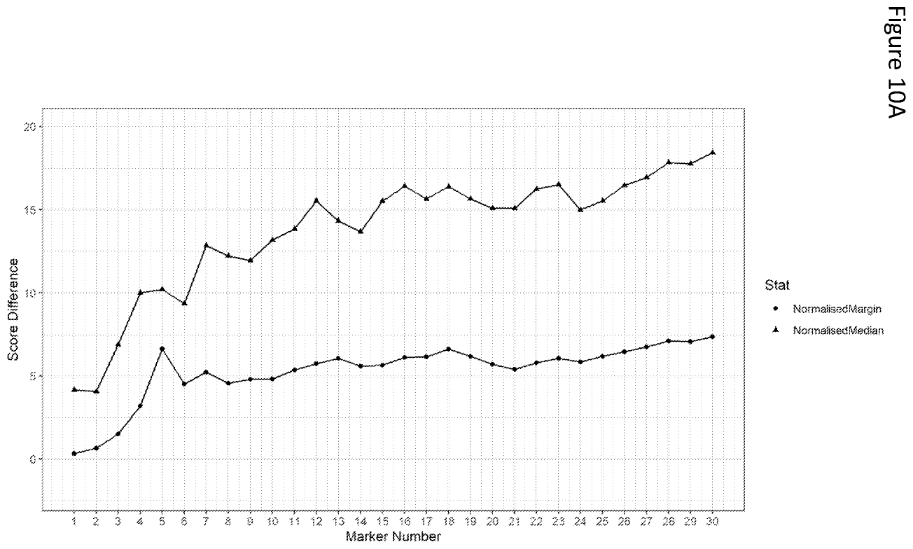

As the marker number increased, the separation between CMMRD and control samples increased, as measured by the difference in score margins and medians between CMMRD and control samples (FIG. 9A). The range of scores within control samples and within CMMRD samples also increased as marker number increased (FIG. 9A). An equivalent analysis that included only the original mononucleotide repeat markers showed a similar trend for increasing range in the data as marker number increases, but the separation of CMMRD from control sample scores is much poorer than when the new markers are included in the ranking (FIG. 9B).

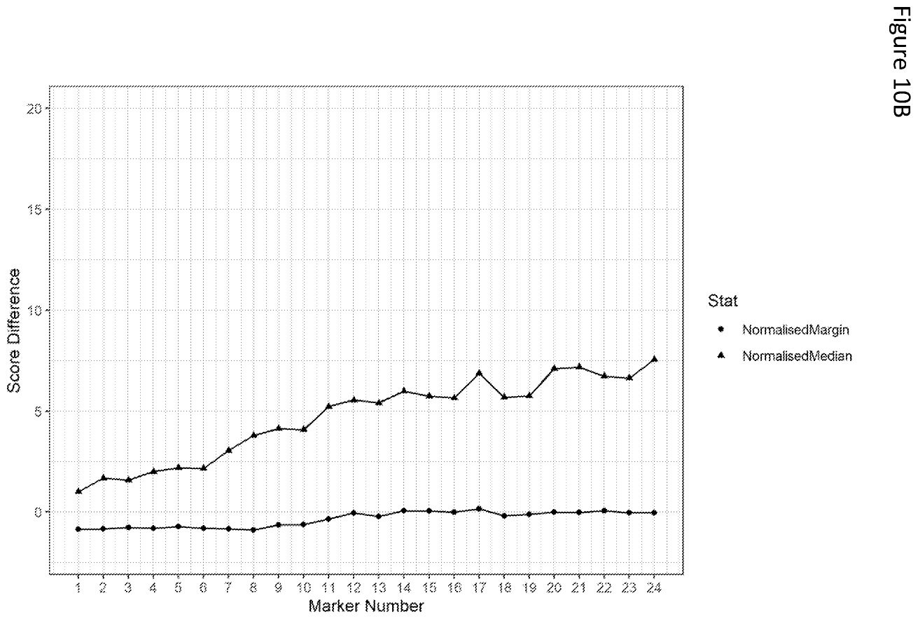

To make the change in the differences in score margins and medians comparable between marker sets, the margin score difference and median score difference were normalised by the control score range for each marker set. A steep increase in the normalised margin and median score differences was observed from the top 1 to top 5 microsatellite markers, followed by a more gradual increase from adding additional markers to the set (FIG. 10A). This suggests that increasing marker number of any of the new markers will increase the ability of the MSI assay and scoring method to detect CMMRD. However, as few as 5 of the new markers will achieve separation of CMMRD from control samples that is almost equivalent to the separation achieved with much greater numbers of microsatellite markers. That so few microsatellite markers can achieve such separation of CMMRD and control samples is novel and unexpected: 5 microsatellite markers is far fewer than the 24 of our original MSI assay (Gallon et al. 2019) or the 186 of the MSI assay of Gonzalez-Acosta et al. 2020, and is equivalent in number to fragment length analysis-based techniques used for tumour MSI analysis. An equivalent analysis that included only the original mononucleotide repeat markers showed a similar trend for increasing normalised differences as marker number increases, but, again, the separation of CMMRD from control sample scores is much poorer than when the new markers are included in the ranking (FIG. 10B).

TABLE 2
Ranking of microsatellite markers from both new and original
microsatellite marker sets using data from the blinded cohort
and known controls. Markers with a ROC AUC <0.90 (using smSequence
RAF as a measure of MSI to detect CMMRD) or a germline variant
frequency >0.05 were excluded. Remaining markers were grouped
by the normalised margin difference ((minimum control RAF −
maximum CMMRD RAF)/range control RAF): Groups included margin
difference >0.00, >−0.25, >−0.50, and <−0.50.
Subsequently, markers were ranked by normalised median difference
((median control RAF − median CMMRD RAF)/range control
RAF) within each group. Original markers are indicated with an asterisk.

| | Normal- | Normal- | Germ- | | |
|---|---|---|---|---|---|
| | ised | ised | line | | |
| | Median | Margin | Variant | | |
| | RAF Dif- | RAF Dif- | Fre- | | |
| Marker | ference | ference | quency | Group | Rank |
| AKMmono10v2 | 2.44 | 0.22 | 0 | 1 | 1 |
| LMmono05v2 | 1.49 | 0.12 | 0.04 | 1 | 2 |
| AKMmono05 | 1.48 | 0.06 | 0 | 1 | 3 |
| AKMmono17v2 | 1.78 | −0.07 | 0 | 2 | 4 |
| LMmono09 | 1.73 | −0.25 | 0.03 | 2 | 5 |
| LMmono03 | 1.71 | −0.12 | 0.03 | 2 | 6 |
| LMmono10v2 | 1.65 | −0.15 | 0.02 | 2 | 7 |
| LMmono04v2 | 1.42 | −0.18 | 0.01 | 2 | 8 |
| AKMmono13 | 1.96 | −0.46 | 0.01 | 3 | 9 |
| AKMmono16 | 1.33 | −0.32 | 0 | 3 | 10 |
| EJmono13v2 | 1.30 | −0.42 | 0.05 | 3 | 11 |
| AKMmono02 | 1.26 | −0.43 | 0.01 | 3 | 12 |
| EJmono16 | 1.22 | −0.49 | 0 | 3 | 13 |
| LMmono01 | 1.18 | −0.45 | 0.03 | 3 | 14 |
| AKMmono03 | 1.16 | −0.33 | 0.01 | 3 | 15 |
| EJmono21v2 | 1.15 | −0.33 | 0.01 | 3 | 16 |
| MSJmono30v2_SNP1 | 0.94 | −0.49 | 0 | 3 | 17 |
| MSJmono46 | 0.93 | −0.26 | 0.01 | 3 | 18 |
| MSJmono10 | 0.83 | −0.43 | 0 | 3 | 19 |
| LMmono16 | 0.82 | −0.37 | 0.02 | 3 | 20 |
| MSJmono44 | 1.91 | −1.00 | 0.01 | 4 | 21 |
| LR36_SNP1* | 1.28 | −1.00 | 0 | 4 | 22 |
| AKMmono08v2 | 1.16 | −0.69 | 0.01 | 4 | 23 |
| EJmono14v2 | 1.05 | −0.69 | 0.01 | 4 | 24 |
| LMmono12 | 1.04 | −0.62 | 0.04 | 4 | 25 |
| EJmono05 | 0.97 | −1.18 | 0 | 4 | 26 |
| MSJmono19ms2 | 0.94 | −1.00 | 0.01 | 4 | 27 |
| GM07_SNP1* | 0.78 | −0.65 | 0 | 4 | 28 |
| AKMmono12 | 0.76 | −0.59 | 0.01 | 4 | 29 |
| LR48_SNP1* | 0.69 | −0.71 | 0 | 4 | 30 |
| LR44_SNP1* | 0.65 | −0.67 | 0 | 4 | 31 |
| MSJmono19ms1 | 0.52 | −1.06 | 0 | 4 | 32 |

*Markers from the original mononucleotide repeat marker set.

### 4. Selected Markers Improve MSI Classification of Colorectal Cancers

### Samples:

50 MSI-H colorectal cancers DNAs (from formalin fixed and paraffin embedded tissue) and 52 MSS colorectal cancers DNAs (from formalin fixed and paraffin embedded tissue) were used.

### Methods:

smMIP-based amplification of 54 of the 62 mononucleotide repeat markers (7 markers missed, but data from the other 54 are sufficient to show marker efficacy), followed by high depth (2000-3000×) amplicon sequencing on a MiSeq (Illumina) was carried out. A custom bioinformatics pipeline to extract microsatellite allele frequencies from the amplicon sequence data was used. Sample classification using the method of our original MSI assay to determine tumour MSI status (Redford et al. PLOS One. 2018 Aug. 29; 13(8):e0203052, DOI:10.1371/journal.pone.0203052, PMID: 30157243; Gallon et al. Hum Mutat. 2020 January; 41(1):332-341, DOI: 10.1002/humu.23906, PMID: 31471937) was performed. The classifier was trained using the same sample cohort of 50 MSI-H and 52 MSS CRCs. A score >0 indicates a higher probability the sample is MSI-H, and a score <0 indicates a higher probability the sample is MSS.

### Results:

All samples had previously been analysed by the original 24 mononucleotide repeat markers using the same method, allowing comparison of the new and original marker sets.

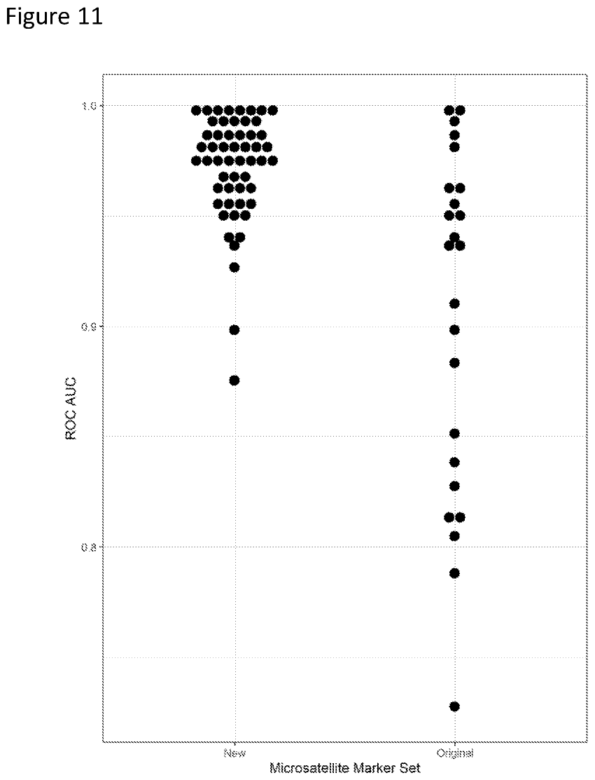

Markers for both new and original marker sets had ROC AUCs calculated for the separation of MSI-H CRCs from MSS CRCs based on read RAF. Potential germline variants were included in the ROC AUC calculation, and therefore the influence of marker polymorphism on its ability to discriminate between MSI-H and MSS CRCs was accounted for in this one value (Table 3A, Table 3B). The RAF ROC AUCs of the new microsatellite marker set were greater than those of the original microsatellite marker set (FIG. 11, Mann Whitney U Test p=8.3×10⁻⁵).

As there was a much greater number of new markers (n=54) than original markers (n=24), the top 24 markers from the new marker set were first identified to allow a fair comparison of classification using these different markers sets. Markers in the new set were ranked using ROC AUC for the separation of MSI-H CRCs from MSS CRCs based on read RAF (as described in the previous bullet point), and the top 24 markers were selected (Table 3A).

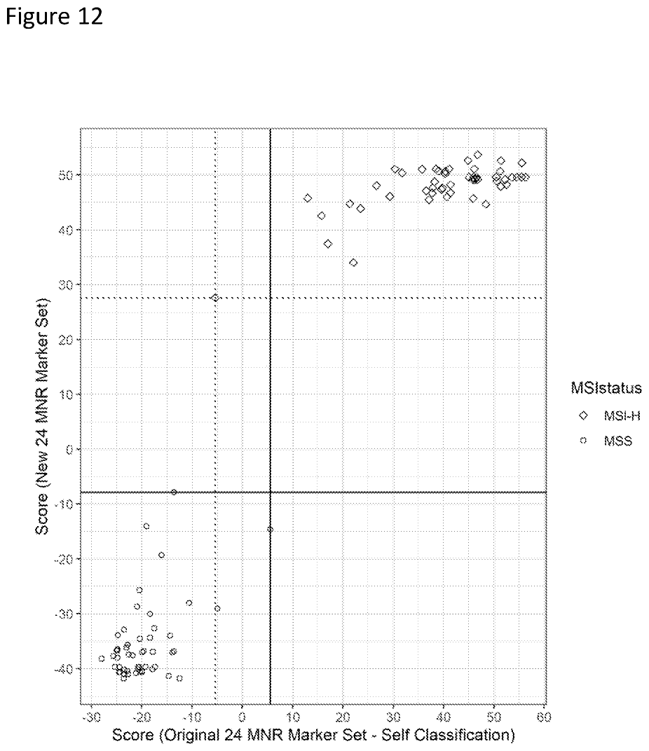

MSI classification of CRCs using the top 24 markers of the new microsatellite marker set had 100% sensitivity (95% CI: 92.9-100.0%) and 100% specificity (95% CIs: 93.2-100.0%), with a clear separation (score difference=35.4) between MSI-H and MSS samples (FIG. 12).

MSI classification of CRCs using the original 24 microsatellite marker set had 98% sensitivity (95% CI: 89.4-100.0%) and 98% specificity (95% CIs: 89.7-100.0%), with overlapping scores (score difference=−11.0) between MSI-H and MSS samples due to misclassification of one MSI-H and one MSS CRC (FIG. 12).

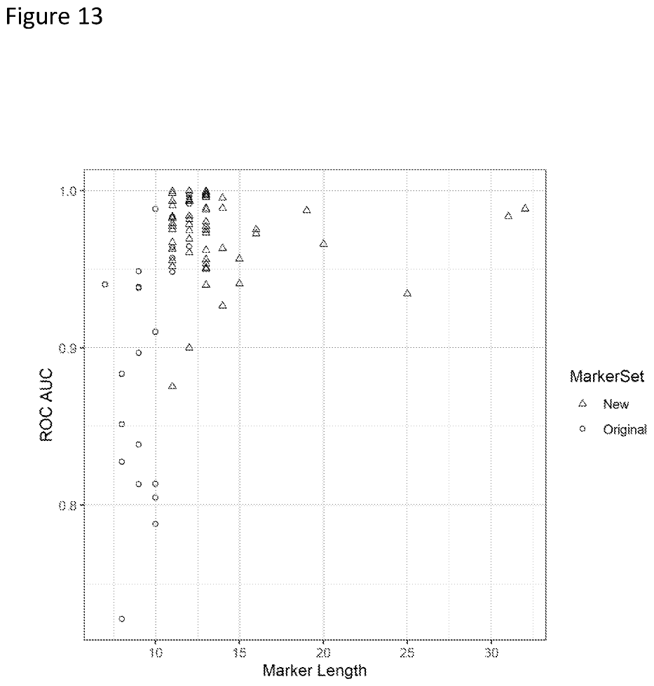

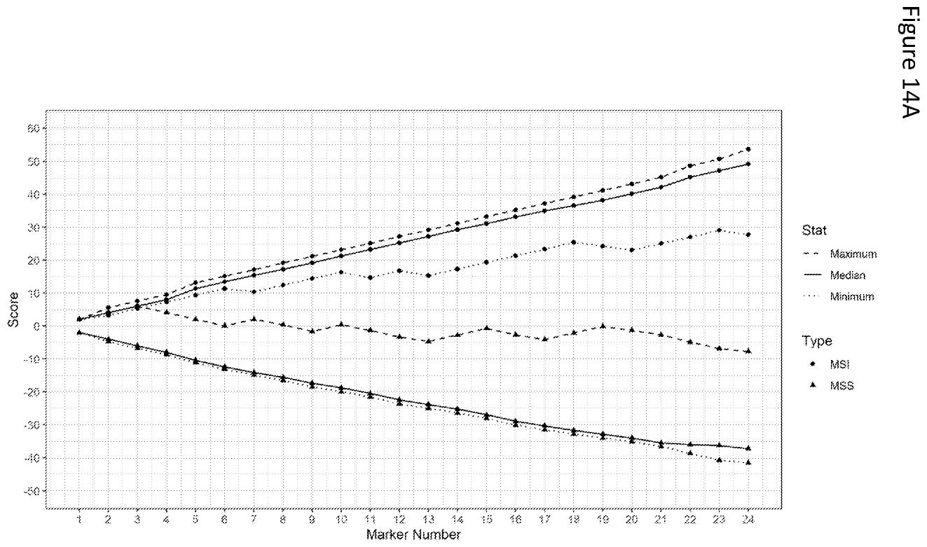

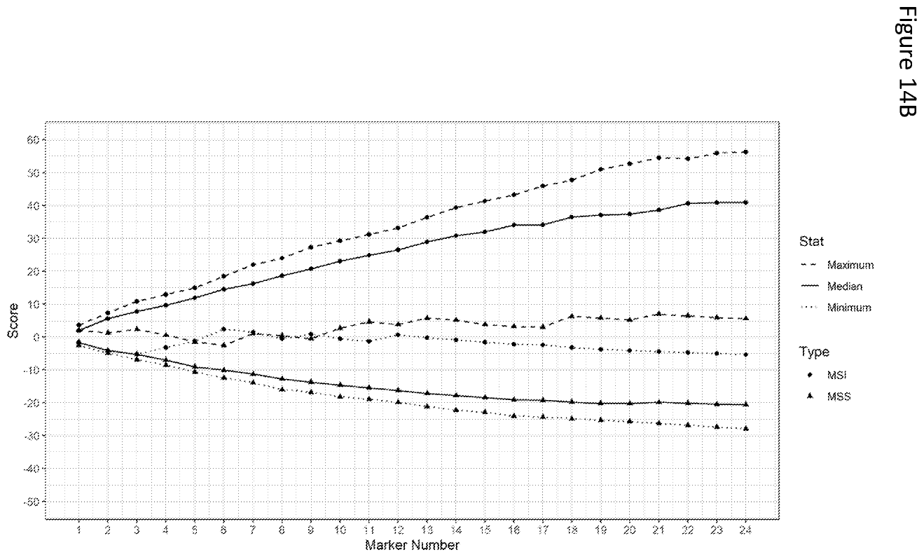

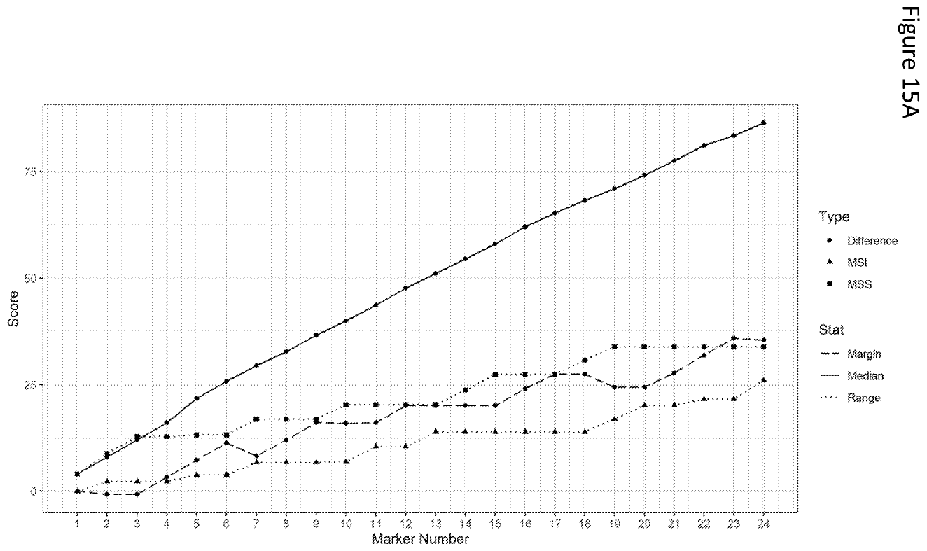

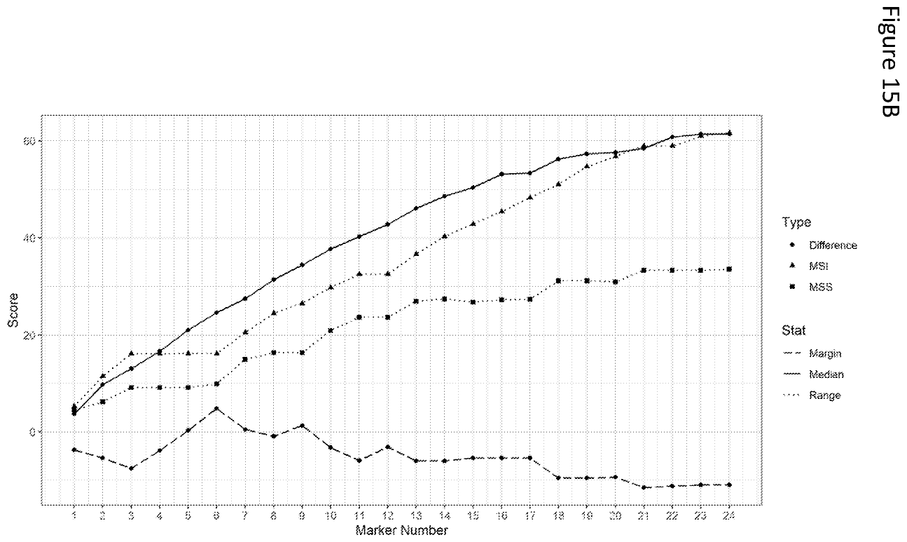

Exploration of microsatellite marker structure across both the original and new mononucleotide repeat markers showed a correlation of ROC AUC (calculated from read RAF of 50 MSI-H and 52 MSS CRCs as described above) and microsatellite length (FIG. 13, Spearman Rho=0.41, p=1.9×10⁻⁴). New mononucleotide repeat markers were generally longer than the original mononucleotide repeat markers (FIG. 13, Mann Whitney U Test p=2.3×10⁻⁹). Unlike for the detection CMMRD by increased MSI in blood, a comparison of the new markers of 11 to 12 nucleotides in length with the original markers of the same length showed no difference in ROC AUC between the two sets using either all 54 of the new markers (Mann Whitney U Test p=0.94), or just the top 24 new markers (Mann Whitney U Test p=0.11). It's worth noting there was less room for improvement on the original markers for these tumour analyses compared to the CMMRD analyses (see page 17 and FIG. 7) as the original markers already had high ROC AUCs for tumour-based MSI testing.

The effect of reducing marker number on MSI assay score distributions was assessed by classifying the 50 MSI-H and 52 MSS CRCs by different marker combinations, starting with the single top ranked marker, then the top two ranked markers, and so on, until all 24 markers were included (Table 3A, Table 3B). With a minimum of 4 of the new microsatellite markers, and any combination with more than 4 markers, separation between all MSI-H and MSS CRCs was achieved. Two MSS CRCs (IDs 296151 and 296213) had consistently high scores across different marker combinations and were responsible for the misclassifications at low marker numbers.

The same two MSS CRCs (IDs 296151 and 296213) that were frequently misclassified by the new marker set also had consistently high scores contributing to misclassifications across nearly all combinations of the original markers. In addition, one MSI-H CRC (ID 215320) had consistently low scores with the original marker combinations, but was correctly classified with all combinations of the new markers. This, again, supports that the method of selection has identified exceptional markers for MSI analysis.

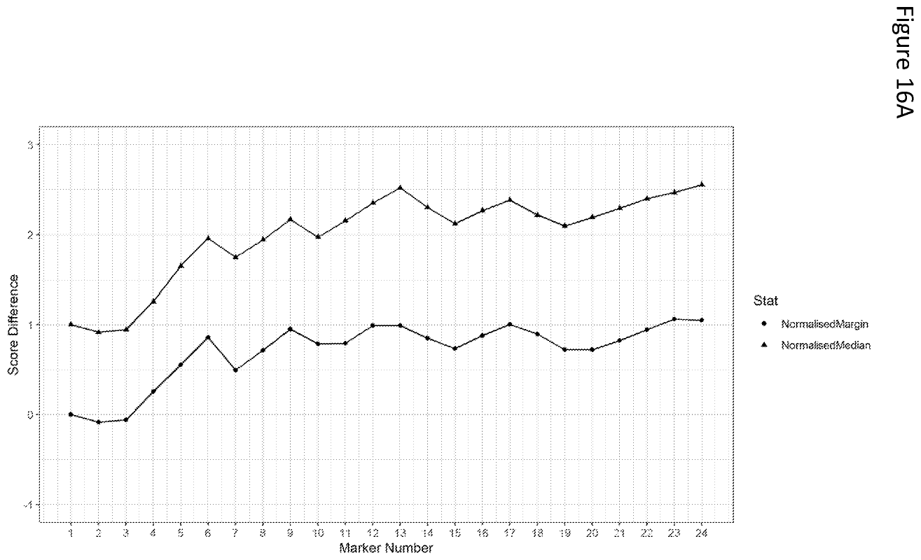

As the marker number increased, the separation between MSI-H and MSS CRC scores consistently increased using the new microsatellite marker set (FIG. 15A). The range of scores within each sample type also increased as marker number increased (FIG. 15A). An equivalent analysis for the original microsatellite markers showed a similar trend for increasing range in the data as marker number increases, but the separation of MSI-H and MSS CRC scores is much poorer than for the new markers: As more of the original markers are added, the score margin between MSI-H and MSS CRCs decreases (FIG. 15B).

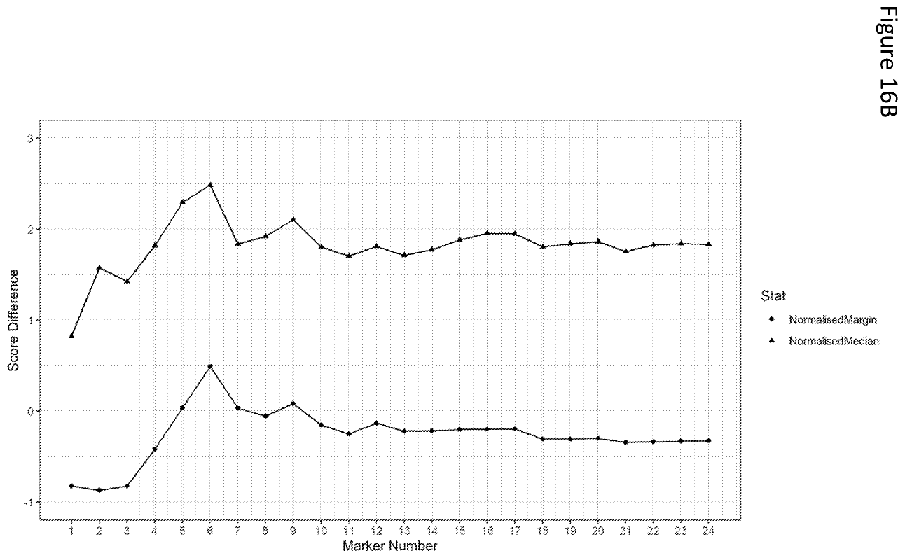

To make the change in the differences in score margins and medians comparable between marker sets, the margin score difference and median score difference were normalised by the MSS CRC score range for each marker set. A steep increase in the normalised margin and median score differences was observed from the top 1 to top 6 microsatellite markers for both new and original microsatellite marker sets (FIGS. 16A and 16B, respectively). For the new microsatellite markers, additional markers steadily increase both normalised margin and median score differences (FIG. 16A). However, this is not true of the original microsatellite markers, as both normalised margin and median score differences initially decrease with additional markers after the top 6, and subsequently level off (FIG. 16B). The normalised margin and normalised median differences for the new microsatellite markers are generally higher than for the original microsatellite markers (compare FIGS. 16A and 16B). We have previously reported that a minimum of 6 microsatellite markers from the original set can be used to achieve accurate MSI classification of CRCs (Gallon et al. 2020), and have reproduced this result in this new cohort of CRC samples for both new and original microsatellite marker sets. However, these data show that a much larger proportion of the new microsatellite markers will improve classification when added into the marker set, further confirming that the method of selection has identified exceptional markers for MSI analysis, which can be used even on their own.

TABLE 3A
Ranking of microsatellite markers from the new microsatellite
marker set using ROC AUCs calculated from read
RAF from 52 MSS and 50 MSI-H CRCs.

| | CRC RAF | |
|---|---|---|
| Marker | ROC AUC | Rank |
| EJmono12_SNP1 | 1.000000 | 1 |
| LMmono05v2_SNP1 | 0.999623 | 2 |
| AKMmono14_SNP1 | 0.999623 | 3 |
| MSJmono22_SNP1 | 0.998869 | 4 |
| EJmono14v2_SNP1 | 0.998492 | 5 |
| MSJmono20_SNP1 | 0.997738 | 6 |
| AKMmono07_SNP1 | 0.996983 | 7 |
| AKMmono05_SNP1 | 0.995852 | 8 |
| LMmono09_SNP1 | 0.995475 | 9 |
| AKMmono02_SNP1 | 0.994344 | 10 |
| AKMmono13_SNP1 | 0.993590 | 11 |
| LMmono08_SNP1 | 0.993590 | 12 |
| MSJmono39_SNP1 | 0.990573 | 13 |
| LMmono03_SNP1 | 0.989065 | 14 |
| AKMmono03_SNP1 | 0.989065 | 15 |
| MSJmono27_SNP1 | 0.988688 | 16 |
| MSJmono46_SNP1 | 0.987934 | 17 |
| MSJmono11_SNP1 | 0.987557 | 18 |
| AKMmono12_SNP1 | 0.984163 | 19 |
| MSJmono40_SNP1 | 0.983786 | 20 |
| EJmono03_SNP1 | 0.983409 | 21 |
| AKMmono17v2_SNP1 | 0.982655 | 22 |
| AKMmono16_SNP1 | 0.981900 | 23 |
| LMmono10v2_SNP1 | 0.980392 | 24 |
| AKMmono10v2_SNP1 | 0.979261 | 25 |
| MSJmono10_SNP1 | 0.978884 | 26 |
| EJmono05_SNP1 | 0.977376 | 27 |
| LMmono12_SNP1 | 0.977376 | 28 |
| EJmono06v2_SNP1 | 0.975490 | 29 |
| EJmono01_SNP1 | 0.975490 | 30 |
| MSJmono23v2_SNP1 | 0.975302 | 31 |
| AKMmono11_SNP1 | 0.974736 | 32 |
| EJmono16_SNP1 | 0.973228 | 33 |
| MSJmono44_SNP1 | 0.972851 | 34 |
| MSJmono38_SNP1 | 0.969457 | 35 |
| MSJmono19ms2_SNP1 | 0.967195 | 36 |
| MSJmono41_SNP1 | 0.966063 | 37 |
| MSJmono26_SNP1 | 0.963424 | 38 |
| LMmono01_SNP1 | 0.963047 | 39 |
| MSJmono30v2_SNP1 | 0.962400 | 40 |
| MSJmono37_SNP1 | 0.960784 | 41 |
| LMmono16_SNP1 | 0.956637 | 42 |
| EJmono02_SNP1 | 0.956259 | 43 |
| AKMmono08v2_SNP1 | 0.955505 | 44 |
| LMmono04v2_SNP1 | 0.953620 | 45 |
| EJmono13v2_SNP1 | 0.952112 | 46 |
| MSJmono15_SNP1 | 0.950980 | 47 |
| LMmono07_SNP1 | 0.950226 | 48 |
| AKMmono06_SNP1 | 0.940799 | 49 |
| MSJmono36_SNP1 | 0.940045 | 50 |
| MSJmono19ms1_SNP1 | 0.934389 | 51 |
| AKMmono22_SNP1 | 0.926848 | 52 |
| AKMmono01v2_SNP1 | 0.900075 | 53 |
| EJmono21v2_SNP1 | 0.875566 | 54 |
| AKMmono04_SNP1 | NA | NA |
| EJmono04_SNP1 | NA | NA |
| HGtetra23ms2_SNP1 | NA | NA |
| MSJcom06ms1_SNP1 | NA | NA |
| MSJcom06ms2_SNP1 | NA | NA |
| MSJmono17_SNP1 | NA | NA |
| MSJmono32_SNP1 | NA | NA |
| MSJmono45_SNP1 | NA | NA |

TABLE 3B
Ranking of microsatellite markers from the original
microsatellite marker set using ROC AUCs calculated
from read RAF from 52 MSS and 50 MSI-H CRCs.

| | CRC RAF | |
|---|---|---|
| Marker | ROC AUC | Rank |
| LR44_SNP1 | 0.997692 | 1 |
| LR52_SNP1 | 0.995769 | 2 |
| IM49_SNP1 | 0.991923 | 3 |
| LR17_SNP1 | 0.988462 | 4 |
| GM07_SNP1 | 0.983462 | 5 |
| LR36_SNP1 | 0.964615 | 6 |
| LR11_SNP1 | 0.964231 | 7 |
| GM14_SNP1 | 0.957308 | 8 |
| GM11_SNP1 | 0.948846 | 9 |
| LR48_SNP1 | 0.948462 | 10 |
| LR49_SNP1 | 0.940385 | 11 |
| IM16_SNP1 | 0.938846 | 12 |
| LR24_SNP1 | 0.938077 | 13 |
| GM29_SNP1 | 0.910385 | 14 |
| LR40_SNP1 | 0.896923 | 15 |
| LR46_SNP1 | 0.883462 | 16 |
| DEPDC2_SNP1 | 0.851538 | 17 |
| GM17_SNP1 | 0.838462 | 18 |
| LR20_SNP1 | 0.827692 | 19 |
| GM26_SNP1 | 0.813654 | 20 |
| LR10_SNP1 | 0.813462 | 21 |
| GM22_SNP1 | 0.805000 | 22 |
| GM01_SNP1 | 0.788077 | 23 |
| GM09_SNP1 | 0.727692 | 24 |

### Example 2

### Introduction

The DNA MMR system is conserved across all three kingdoms of life. Primarily, it mediates the repair of base-to-base mismatches and small insertion-deletion loops generated during DNA replication, as well as a variety of base modifications such as cytosine deamination and guanine methylation, by excision of the affected DNA strand for resynthesis whilst signalling to the wider DNA damage response (DDR). MMR function can be lost in a wide variety of neoplasia, affecting approximately 1 in 4 endometrial cancers (ECs) and 1 in 7 CRCs. MMR deficient tumours are often hyper-mutated, with >10 mutations per megabase, and display high levels of MSI, a molecular phenotype defined as the accumulation of insertion and deletion (indel) mutations in short tandem repeat sequences scattered throughout the genome. An elevated mutation rate in the absence of MMR has also been demonstrated using human cell line, mouse, yeast, and bacterial models, and has been proposed to drive tumorigenesis through secondary mutation of onco- and tumour suppressor genes. Indeed, functional studies have demonstrated that frameshifts caused by coding microsatellite indels promote malignant cell growth. Furthermore, an over-representation of disruptive C>T transitions associated with defective MMR, and coding microsatellite frameshifts in the APC tumour suppressor gene has been observed in MMR deficient compared to proficient CRCs. The distinct patterns of recurrent coding microsatellite frameshift mutations between different tumour types also suggests tissue-specific positive selection of MMR deficiency-associated mutations during tumorigenesis.

Individuals with LS carry a germline pathogenic variant in one of the four principle MMR genes, MLH1, MSH2, MSH6, or PMS2, and have an increased risk of cancer, in particular CRC, EC, and other tumours of the gastrointestinal and genitourinary tracts. LS is one of the most common hereditary causes of cancer, affecting approximately one in 300 individuals in the general population. CMMRD is a far rarer childhood cancer syndrome caused by germline variants affecting both alleles of MLH1, MSH2, MSH6, or PMS2, with an estimated birth incidence of one per million. The loss of MMR function in all constitutional tissues is associated with an exceptionally high cancer risk, with a median age of onset less than 10 years. This includes LS cancers, which occur in approximately one third of cases, and, more commonly, high grade brain tumours and haematological malignancies. CMMRD is also associated with several non-neoplastic features, the most distinctive of which are café au lait macules (CALM) and skin-fold freckling reminiscent of neurofibromatosis type 1 (NF1). Other features include localised skin hypopigmentation, defective immunoglobulin class switch recombination, pilomatrixoma, and multiple developmental venous anomalies. Presentation may depend on which MMR gene is affected in the patient's germline. In a review of 146 published cases, haematological malignancies were more prevalent in MLH1- or MSH2- than PMS2-associated CMMRD (p=0.04), whilst the opposite was true of brain tumours (p=0.01). Furthermore, MLH1 or MSH2-associated CMMRD cancers tended to occur earlier, which correlates with the earlier onset of MLH1- and MSH2-associated LS.

Given the role of MMR deficiency in tumour progression, it is possible the malignant and non-malignant clinical features of CMMRD are, to varying extent, linked to an increased constitutional mutation rate. Increased MSI in non-neoplastic tissues is a highly specific feature of CMMRD, that is detectable by high-depth amplicon sequencing or low-pass whole genome sequencing but not by traditional MSI analysis methods. Sequencing-based microsatellite analysis can quantify the proportion of microsatellites demonstrating instability and the frequency of variant alleles at each microsatellite (collectively referred to here as MSI-burden) to approximate constitutional mutation rate.

Previously, using high-depth amplicon sequencing, the inventors observed a relatively low MSI-burden in the peripheral blood leukocytes (PBLs) of CMMRD cases homozygous for a hypomorphic PMS2 variant (c.2002A>G p. (Ile668Val)) typified by an attenuated phenotype more similar to early-onset LS than classical CMMRD. This observation suggested constitutional MSI-burden may correlate with MMR genotype and/or CMMRD disease phenotype. However, more comprehensive analyses were precluded by the limited cohort-size of 32 patients and an MSI assay that only minimally separated CMMRD samples from controls. Further exploration of such correlations could broaden our understanding of how MMR deficiency contributes to malignant transformation, aid variant interpretation, and allow risk stratification to guide clinical management of CMMRD.

Here the inventors aimed to enhance methods to quantify constitutional MSI-burden, and subsequently explore its association with CMMRD genotype and phenotype using a relatively large cohort. One limitation of the previous method was its use of markers selected for MSI analysis of tumours as dysregulated replication, a possible mutator phenotype, and a common lineage, whereby cancer subclones are more likely to share mutations than the thousands of clones represented in healthy peripheral blood, may contribute to different mechanisms and frequencies of microsatellite mutation in cancers compared to non-neoplastic blood. Therefore, new MSI markers selected for instability in blood were desirable. Here, the inventors identified potentially informative MSI markers from high depth genome sequencing of CMMRD blood, and used amplicon sequencing to refine a panel of markers with highest sensitivity to MMR deficiency and to quantify constitutional MSI-burden in over 50 CMMRD patients.

### Materials and Methods

### Patient Samples and Ethical Approval

Anonymised CMMRD PBL gDNAs were sourced from the Medical University of Innsbruck, Innsbruck, Austria (MUI), the University of Manchester, Manchester, UK (UM), the Gustave Roussy Cancer Campus, Villejuif, France (GR), the Institut Curie, Université de Recherche Paris Sciences et Lettres, Paris, France (IC), and the Cancer Centre de Recherche Saint-Antoine, Sorbonne University, Paris, France (CRSA). MMR variants were classified according to InSIGHT criteria v2.4 and reference to ClinVar and InSIGHT databases. For patients with one or more VUS, the diagnosis had been confirmed by assessment of MMR function in non-neoplastic tissues, including assays of germline/constitutional MSI, and/or ex vivo MSI and methylation tolerance. PBL gDNAs from eight patients with a CMMRD-like phenotype but who tested negative for germline MMR pathogenic variants were sourced from MUI. Patient samples were analysed with consent and ethical approval by the review boards of the respective centres.

Anonymised control PBL gDNAs were extracted from discard blood samples of patients tested for non-cancer related conditions from Newcastle-upon-Tyne Hospitals NHS Foundation Trust, Newcastle-upon-Tyne, UK (NuTH) and MUI, following ethical review by the NHS Health Research Authority (REC reference 13/LO/1514) and the MUI review board, respectively.

Anonymised genetically-diagnosed LS PBL gDNAs were sourced from the CaPP3 clinical trial (ISRCTN16261285) biobank with participant consent for sample-use in research, and analysed following an ethical review by the NHS Health Research Authority (REC reference 13/LO/1514).

PBL samples were divided across three cohorts. High quantity (>2 μg) and quality samples from three CMMRD patients (2 MUI, 1 UM), one LS carrier (CaPP3), and two controls (NuTH) were whole genome sequenced. Eight CMMRD (MUI) and 38 control (NuTH) samples were analysed in a pilot cohort. Fifty-seven CMMRD (31 MUI, 9 GR, 4 IC, and 13 CRSA), eight CMMRD-negative (MUI), and 43 control (MUI) samples were analysed as a blinded cohort, alongside 80 known controls (30 MUI, 50 NuTH) to provide reference samples for MSI scoring and 40 LS samples (CaPP3).

CRC samples were sourced from NuTH as 10 μm FFPE tissue curls of resected tumours or pre-extracted gDNAs from non-fixed endoscopic biopsies, following an ethical review by the NHS Health Research Authority (REC reference 13/LO/1514). FFPE CRC gDNAs were extracted using GeneRead DNA FFPE Kit (QIAGEN). Eight MMR deficient and 8 MMR proficient CRC endoscopic biopsies were included in a pilot cohort, and a further 96 MMR deficient and 96 MMR proficient FFPE resected CRCs were analysed to train and validate a naïve Bayesian classifier.

### Genome Sequencing and Variant Analysis

Samples were prepared for whole genome sequencing by 3 cycle PCR amplification using the NEBNext® Ultra™ II DNA Library Prep Kit for Illumina (New England Biolabs), and were sequenced to >120× coverage on a NovaSeq (Illumina). Reads were aligned to human reference genome build hg19 using BWA mem and BAM files generated using SAMtools view, sort, and index. Variants were called by a somatic variant calling pipeline and panel of reference control genomes using GATK 4 MuTect2, followed by GetPileupSummaries, CalculateContamination, and FilterMutectCalls, with PCR_indel_model set to NONE. Variants were classed as germline if the probability of variant allele frequency equalling the 1:1 or 1:0 ratio expected of a germline variant was >10⁻⁷.

For MSI marker selection, microsatellite variants flagged as germline and/or identified in the panel of reference genomes were excluded. Variants annotated as clustered events, multiallelic, slippage, or PASS, and where the total variant allele frequency was <0.25 (to further exclude potential germline variants) were retained and visually inspected using IGV. Microsatellites with variants captured by high quality read-alignments, not embedded within conserved repetitive elements, and that had higher variant allele frequencies in CMMRD patients than in controls were selected for further assessment by amplicon sequencing.

### Single Molecule Molecular Inversion Probe Design and Amplicon Sequencing

Single molecule molecular inversion probes (smMIPs) were designed using MIPgen to amplify MSI markers with capture sizes between 100 bp and 160 bp, and a molecular barcode of 4N at both extension and ligation arms.

MSI markers were amplified from samples using a published smMIP and high fidelity polymerase-based protocol. Amplicons were purified using AMPure XP beads (Beckman Coulter), quantified using a QuBit fluorometer 2.0 (Invitrogen), diluted to 4 nM using 10 mM pH8.5 Tris-HCl buffer, and pooled into 4 nM sequencing libraries. Sequencing libraries were sequenced using custom sequencing primers on a MiSeq (Illumina) to a target depth of 5000×, following manufacturer's protocols.

### Microsatellite Amplicon Sequence Analysis and Microsatellite Instability Scoring

Amplicon sequence reads were aligned to human reference genome build hg19 using BWA mem and further processed and analysed as previously described. In brief, to reduce PCR and sequencing error for low frequency variant detection, reads sharing the same molecular barcode were grouped and the microsatellite length represented in the majority of reads was defined as the single molecule sequence (smSequence) for each group. Groups containing only one read or without a majority were discarded. Microsatellite reference allele frequencies (RAFs) in smSequences were used to generate an MSI score (equivalent to MSI-burden) for each sample by comparison to RAFs of 80 known control samples. For any sample, MSI markers with a RAF <0.75 (probable germline variants) or with <100 smSequences were excluded from MSI scoring.

### Statistical Analyses and Data Availability

All analyses used R version 4.0.2. Comparisons of two sample groups used the Mann-Whitney test. Comparisons of more than two sample groups used the Kruskal-Wallis test. Correlation of variables where a linear relationship could or could not be assumed used Pearson's R or Spearman's rho, respectively. Confidence intervals for sensitivity and specificity estimates used a binomial distribution.

Genome sequence BAM and amplicon sequence FASTQ files are available from the European Nucleotide Archive using Study IDs PRJEB39601 and PRJEB53321, respectively.

### Results

### Genome Sequencing of Blood Identifies High Sensitivity MSI Markers

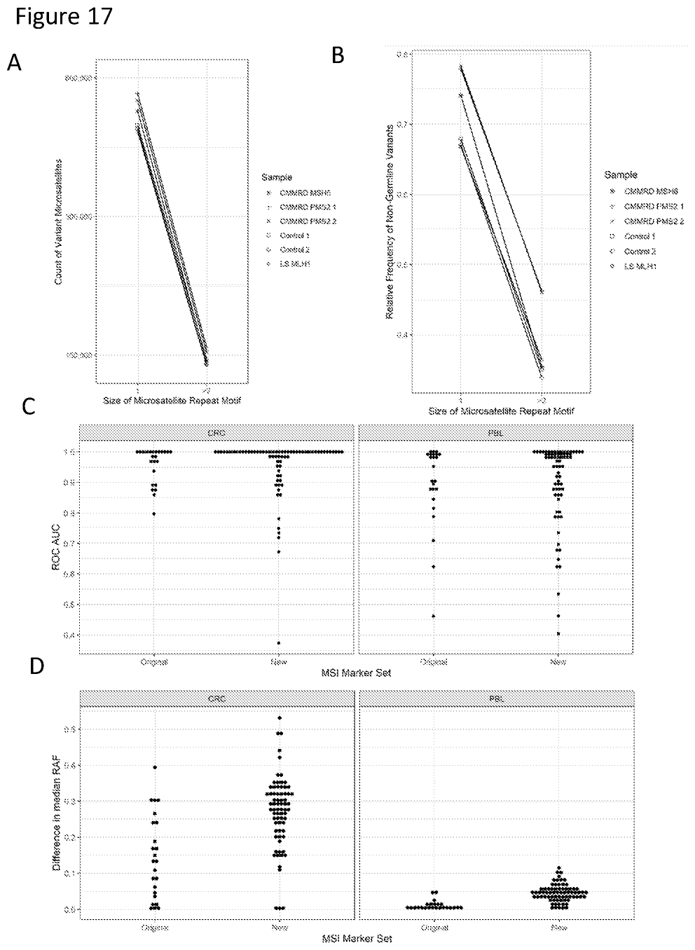

Three CMMRD (two PMS2- and one MSH6-associated), one LS (MLH1-associated), and two control blood samples were whole genome sequenced. An LS sample was included as highly sensitive MSI analysis and single-base-mismatch repair assays have previously detected reduced MMR function in blood and cell lines with one dysfunctional MMR allele. There was a marginal increase in the frequency of mononucleotide repeat (MNR) variants in both PMS2-associated and MSH6-associated CMMRD bloods relative to control and LS bloods, but an increase in variants of longer motif microsatellites was only observed in the PMS2-associated CMMRD bloods (FIG. 17A). These variants include PCR error, sequencing error, germline variants, and somatic variants. To enhance the somatic signal, probable germline variants were identified, and the relative frequency of non-germline variants was assessed. In both PMS2-associated and MSH6-associated CMMRD bloods there was an increase in the relative frequency of non-germline MNR variants compared to LS and control bloods, but, again, an increase in longer motif microsatellites was observed only in the PMS2-associated CMMRD bloods (FIG. 17B). This is consistent with the role of MSH6 in the repair of single nucleotide indels, mismatches, and modifications, but not multiple nucleotide indels.

Microsatellites with a potential to enhance MSI analysis in blood were selected from the blood genome sequence data (see Methods), with review of over 2000 microsatellites, the majority of which were 11-16 bp A-homopolymers. Since MSH6 deficiency causes 20% of CMMRD and MNR instability was increased in both MSH6- and PMS2-associated CMMRD samples in genome sequence analysis, 121 MNRs were short-listed as candidate MSI markers for further assessment by amplicon sequencing. These were smMIP amplified and sequenced from three control bloods, and 91 smMIPs (covering 98 candidate markers) generated read counts >10% of the median read depth and were taken forward. The ability of candidate markers to discriminate between MMR deficient and MMR proficient tissues was assessed by smMIP-amplicon sequencing a pilot cohort of eight CMMRD and 38 control blood gDNAs, as well as eight MMR deficient and eight MMR proficient CRC gDNAs. All except seven control samples had been previously analysed using the 24 tumour-derived MNRs of the original MSI assay, allowing comparison of marker sets. Twenty-seven of the 98 new blood-derived MSI markers were excluded as >10% of the pilot PBL samples had a RAF <0.75 indicative of a germline length variant (see Methods). There was no difference in the receiver operator characteristic (ROC) area under curve (AUC) values based on microsatellite RAF between the remaining 71 new and 24 original MSI markers to detect MMR deficiency in either pilot CRCs (p=0.439) or pilot PBLs (p=0.530, FIG. 17C). However, the difference between the median RAFs of MMR deficient and MMR proficient samples was significantly greater for the new markers in both CRCs (p=1.81×10⁻⁵) and in PBLs (p=2.18×10⁻⁸, FIG. 17D), indicating they are more sensitive to MMR deficiency. Based on these data and visual inspection of microsatellite allele distributions, the candidate markers were refined to a panel of the most discriminatory 32 MNRs (Table H).

### New MSI Markers Enhance the Detection of CMMRD

The 32 new MSI markers were amplified and sequenced from 80 control PBL gDNAs to provide a reference for MSI scoring, and a blinded cohort of 57 CMMRD, 8 CMMRD-negative (patients with a CMMRD-like phenotype but no germline MMR variants), and 43 control PBL gDNAs. Forty LS PBL gDNAs (10 for each MMR gene) were also analysed to investigate if increased MSI in blood is specific to biallelic loss of MMR function. One sample from the blinded cohort failed to amplify, and was later revealed to be a CMMRD case. All other sample amplicons were sequenced and an MSI score generated for each. Markers with low (<100) smSequence counts were observed in only four samples: Two had a single low count-marker, whilst the others had <100 smSequences in ≥17 MSI markers with equivalent results upon repeat amplification and sequencing, suggesting poor sample quality. On un-blinding, these two samples were revealed to be CMMRD cases.

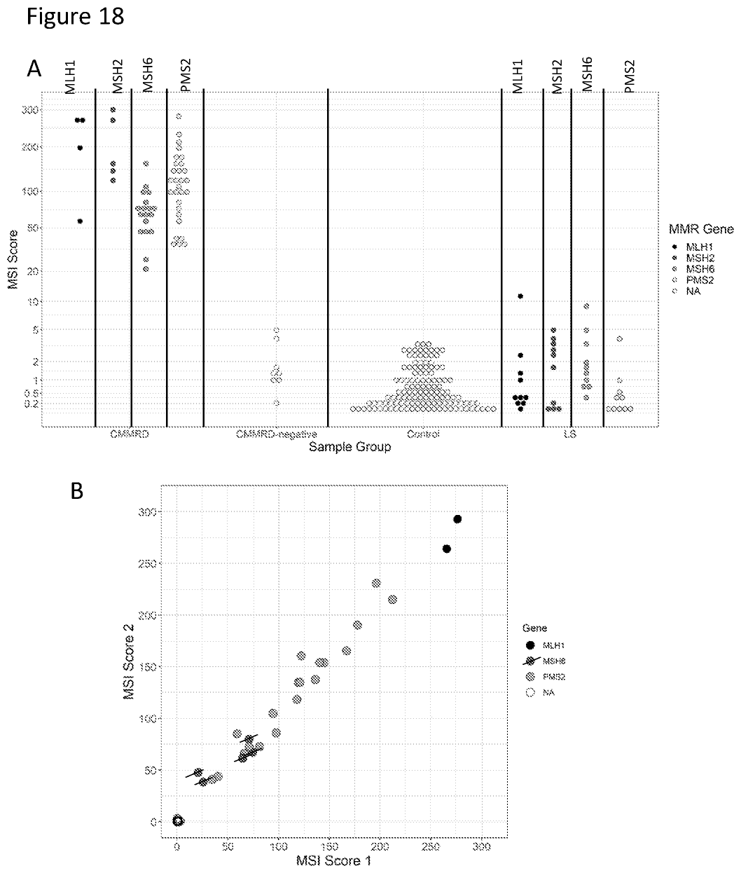

The blood MSI score identified CMMRD with 100% sensitivity (56/56; 95% CI: 93.6-100.0%) and 100% specificity (171/171; 95% CI: 97.9-100.0%), including the two CMMRD samples with exceptionally low smSequence counts, and with a clear separation from control, LS, and CMMRD-negative samples (FIG. 18A). MSI score was associated with affected MMR gene (p=1.15×10⁻³); patients with MSH6 deficiency had significantly lower MSI scores than patients with MSH2 deficiency (p=2.38×10⁻⁴) or PMS2 deficiency (p=6.01×10⁻³), and a trend for lower MSI scores than patients with MLH1 deficiency (p=5.30×10⁻², multiple testing significance at p<1.67×10⁻²). LS MSI scores were not significantly different from controls (p=0.169), but it was notable that six (3.7-11.3) were greater than the highest control (3.6). CMMRD-negative samples generally had higher MSI scores than controls (p=0.0188). However, small but significant differences were observed between controls of different amplification and sequencing batches (p=1.23×10⁻⁸, FIG. 18), and 7/8 CMMRD-negative samples were analysed in a single batch. For these seven, there was no significant difference in MSI score compared to controls from the same batch (p=0.0958). However, it was notable that two CMMRD-negative MSI scores (4.1, 5.3) were greater than the highest control (3.6). As these high scoring LS and CMMRD-negative samples had much lower MSI scores than the CMMRDs they were not analysed further. To assess MSI assay reproducibility, residual DNA samples available from 26 CMMRD patients and 33 controls were re-amplified, sequenced, and scored, and a strong correlation was found between initial and repeat MSI scores (R=0.994, p<10⁻¹⁵, FIG. 18B).

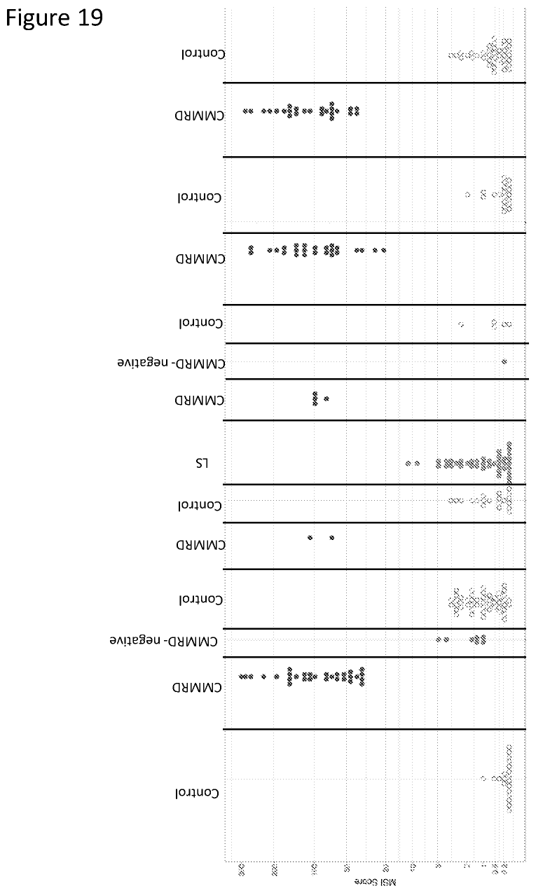

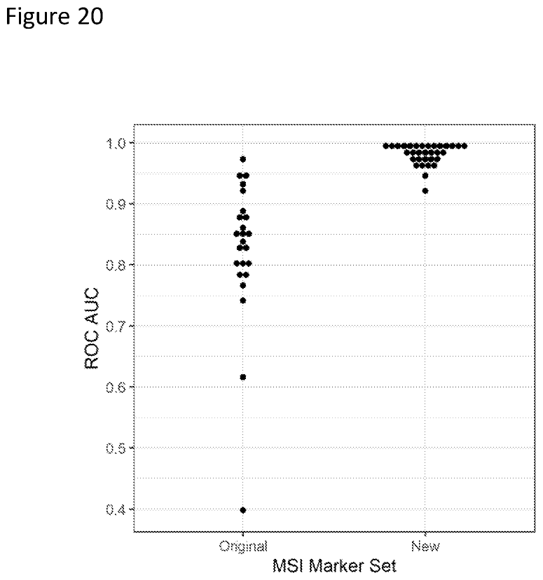

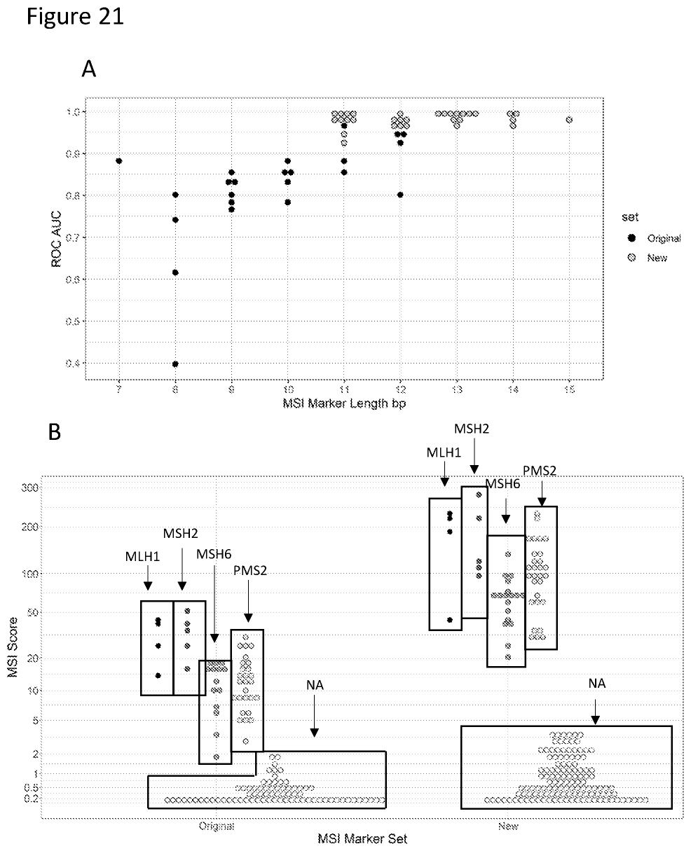

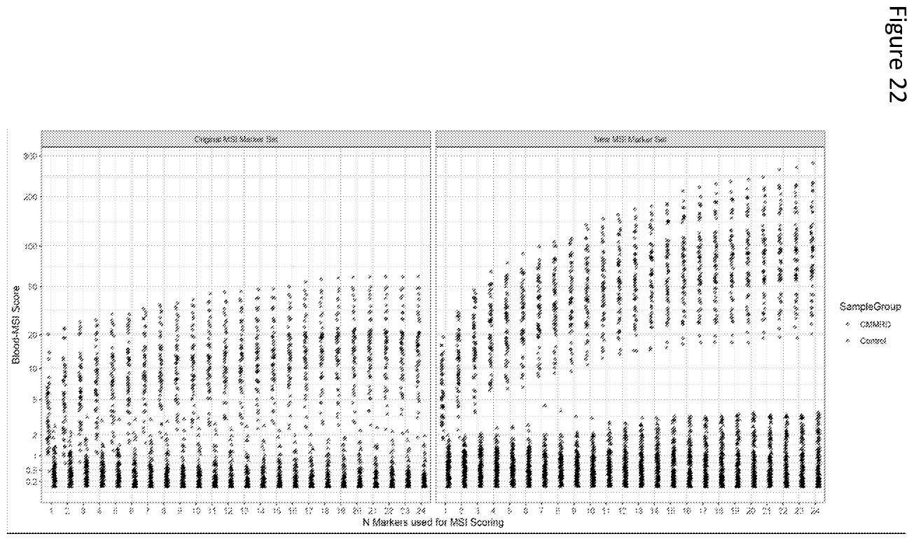

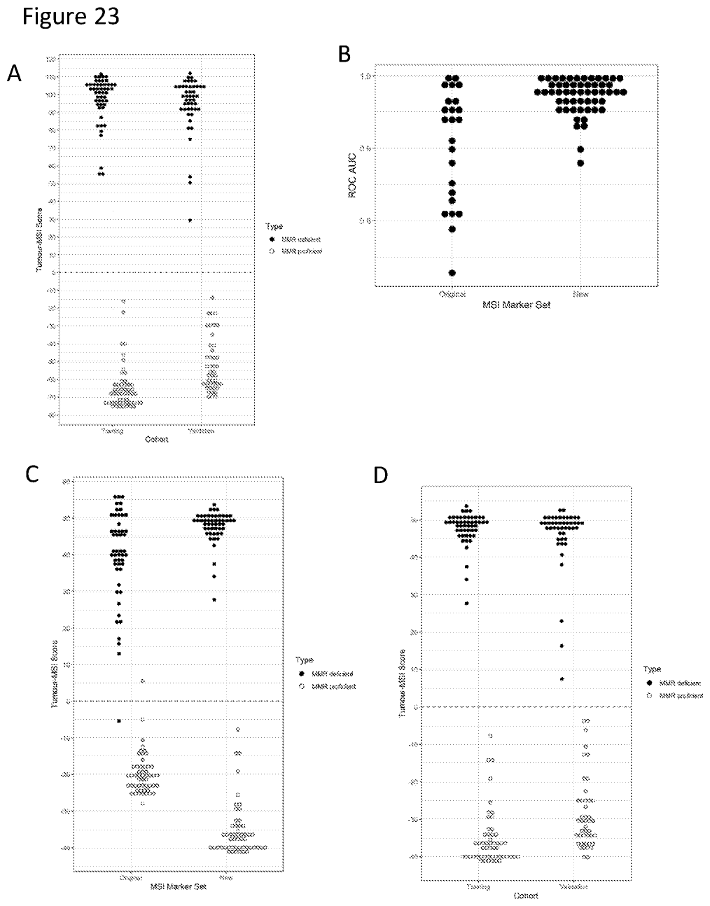

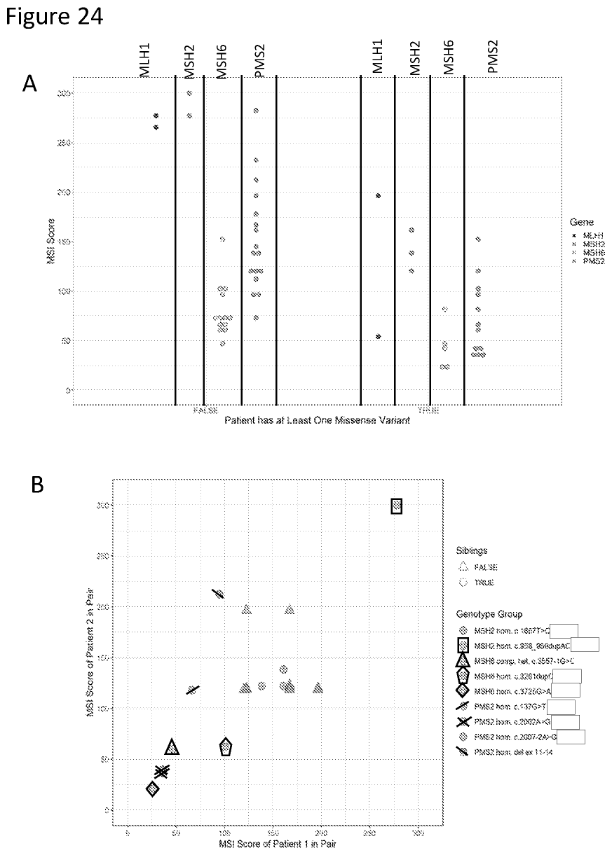

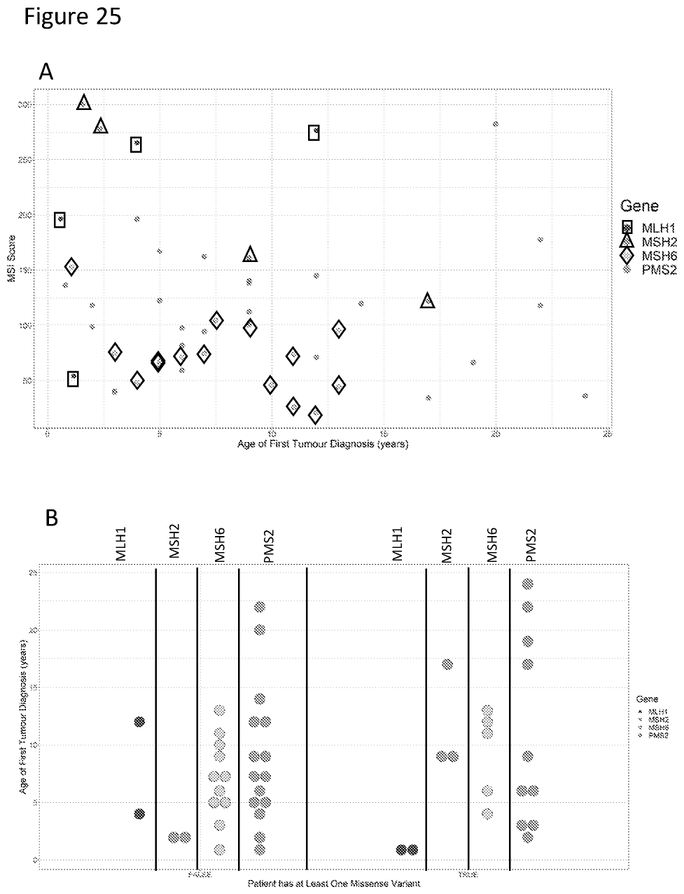

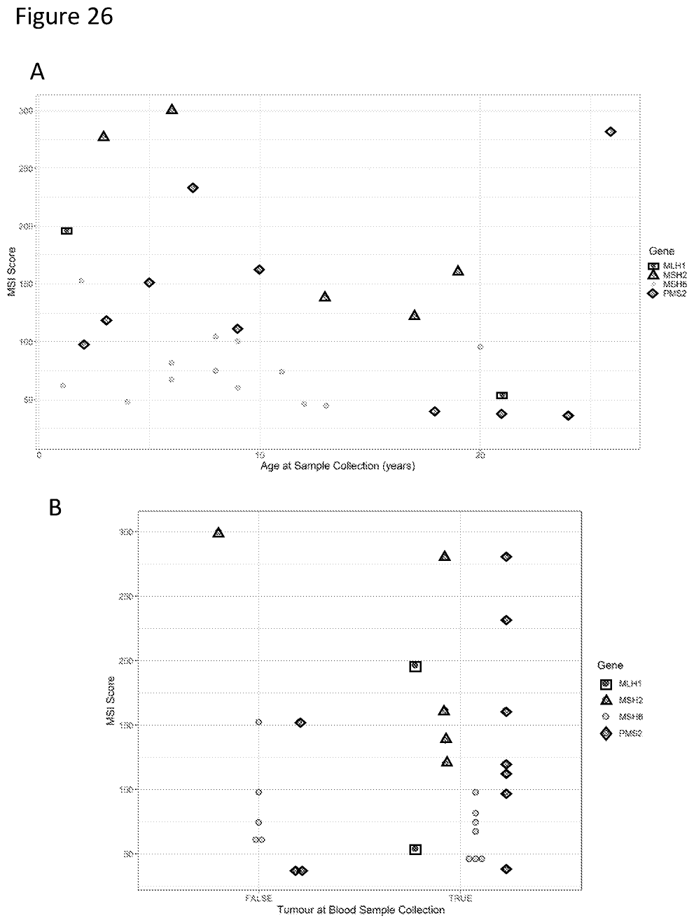

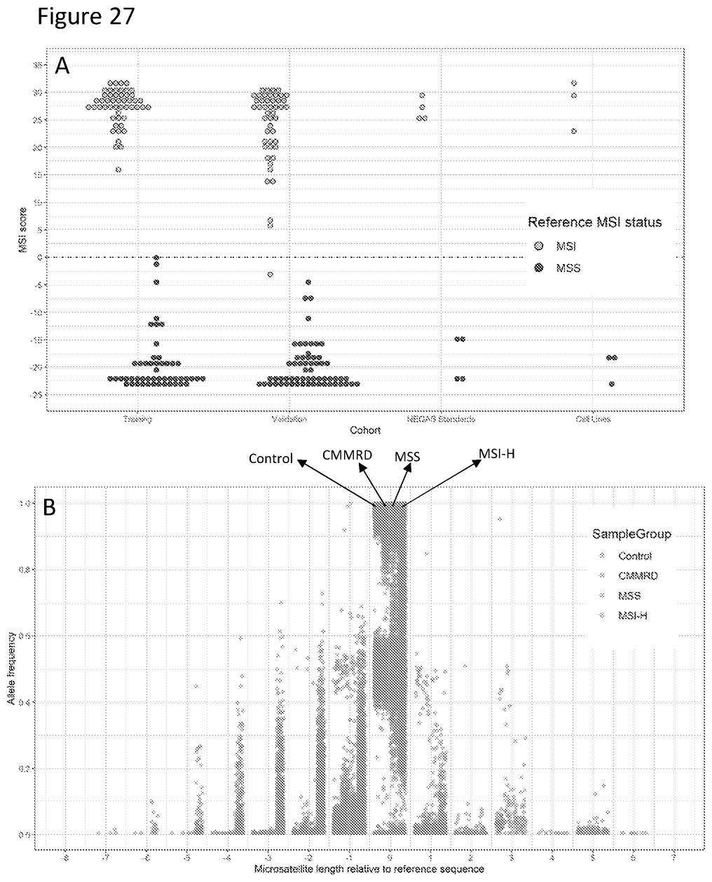

Fifty CMMRD and 75 control samples were also analysed using the original 24 MSI markers. The new MSI markers had greater RAF-based ROC AUCs for CMMRD detection than the original set (p=9.00×10⁻¹⁴, FIG. 19). The new MSI markers were longer (range 11-15 bp versus 7-12 bp, p=1.93×10⁻⁷) and there was a strong positive correlation between marker length and ROC AUC (rho=0.730, p=1.79×10⁻¹⁰). However, comparing markers of equivalent size (11-12 bp) found higher ROC AUCs for the new markers than the original (p=2.52×10⁻⁴, FIG. 21A). The new MSI markers were ranked by RAF ROC AUC to separate CMMRD from control samples (Table H) and the most discriminatory 24 new MSI markers maintained a large MSI score separation of 15.3 between CMMRD and control samples, compared to the 0.1 MSI score overlap when using the original 24 MSI markers (FIG. 21B). Using only three new MSI markers gave 100% accurate CMMRD detection (FIG. 22). The new MSI markers also enhanced MSI classification of CRCs compared to the original set (FIG. 23A-D) and there was a strong correlation between their RAF ROC AUCs in CRCs compared to bloods (rho=0.715, p=9.01×10⁻⁵).

CMMRD Constitutional MSI-Burden is Associated with MMR Genotype but not Age of Tumour Onset

There was a breadth of MSI scores between CMMRD patients with deficiency of the same MMR gene suggesting potential genotype or phenotype correlations with constitutional MSI-burden. CMMRD patients with one or more missense MMR variant had significantly lower MSI scores than those without (p=8.81×10⁻⁴, FIG. 24A), whilst the frequency of missense variants was equivalent between MMR genes indicating this was not due to an over-representation of missense variants in any one gene group (p=0.55). To further assess whether MMR variants associate with constitutional MSI-burden, pair-wise comparisons of MSI score between patients sharing the same genotype were made. This included twelve pair-wise comparisons between siblings of eight CMMRD families, and ten between five unrelated patients homozygous for the recurrent PMS2 c.2007-2A>G variant, finding MSI scores were strongly correlated between pairs (R=0.744, p=7.13×10⁻⁵, FIG. 24B).

A clinical history of tumour diagnoses was available for CMMRD patients. Five patients had no cancer history, and for another the age of tumour diagnosis was unknown. Despite the strong genotype correlations with constitutional MSI-burden, no correlation was observed between age of first tumour and MSI score overall (Rho=−0.154, p=0.287, FIG. 25A), or for subgroup analysis of MSH6-deficient cases (Rho=−0.342, p=0.195) and PMS2-deficient cases (Rho=−1.31×10⁻², p=0.95). It is possible that constitutional MSI-burden is associated with the onset of specific tumour types as both sporadic and CMMRD-related brain and haematological malignancies have a reduced frequency of MSI compared to cancers within the LS spectrum. However, no correlation was found between MSI score and the age of onset of brain tumours (Rho=−0.167, p=0.318), haematological malignancies (Rho=−0.285, p=0.268), or LS-associated tumours (Rho=−0.143, p=0.582). There was also no association of age of first tumour with affected MMR gene (p=0.483) or with whether the CMMRD patient had at least one missense MMR variant (p=0.457, FIG. 25B).

Other factors that might affect constitutional MSI-burden include age at sample collection contaminating tumour DNA. Age at sample collection was not correlated with MSI score among 30 CMMRD patients with data available (Rho=−0.310, p=9.9×10⁻², FIG. 26A) but was correlated with age of first tumour (R=0.727, p=3.87×10⁻⁵) as expected given CMMRD diagnoses will typically be made at presentation of a malignancy. Similarly, MSI score was not associated with age at sample collection in 50 controls with data available (p=0.652). For 27 CMMRD patients it was also known if a tumour was present at the time of sample collection; the MSI scores of the 18 patients with a tumour were equivalent to those without (p=0.495, FIG. 26B).F

### Discussion

Novel MSI markers were selected in this study from blood WGS to enhance an existing amplicon sequencing-based MSI assay, achieving excellent separation of CMMRD samples from controls. Sequencing-based MSI analysis to detect CMMRD has now been demonstrated with a variety of methods. However, the method used here has a particularly low cost and is scalable from functional testing of a few samples to high throughput screening, as demonstrated when screening for CMMRD in cancer-free children with NF1-like phenotypes but negative for NF1 or SPRED1 germline variants. Functional assays also support ambiguous genetic test results, such as MMR VUS and analysis of PMS2 (the MMR gene affected in the majority of CMMRD patients), which otherwise needs specialist techniques to avoid its pseudogenes. The inventors' results provide data to support reclassification of 17 MMR VUS as pathogenic, at least in the context of CMMRD. The new MSI markers were found to be longer than the original set, ranging between 11 bp and 15 bp, which is equivalent to the most sensitive and specific A-homopolymers identified in TCGA tumour exome sequencing data. This suggested that a microsatellite's diagnostic utility may simply be a function of its length. However, a comparison of 11-12 bp markers showed the new blood-derived MSI markers have significantly higher ROC AUCs than the original tumour-derived set, confirming this new selection had identified exceptional markers. The new MSI markers also enhanced detection of MMR deficiency in CRCs, suggesting that they will be sensitive irrespective of tissue despite our initial hypothesis that some microsatellite markers may be more sensitive in blood than in tumours. However, the original tumour-derived set analysed here had also been selected to be ≤12 bp and have a SNP within 30 bp, and so these differences in selection criteria may mask tissue specificity.

A CMMRD patient's MSI score was associated with their genotype. Previously, a reduced MSI-burden in MSH6- versus MSH2-associated CMMRD cases using an alternative amplicon sequencing assay was found. Here, the inventors have shown that this extends to PMS2-associated CMMRD and that there is a similar trend comparing MSH6- to MLH1-associated CMMRD. A reduced MSI-burden of MSH6- compared to PMS2-associated CMMRD was also observed in our genome sequence data, and is consistent with genome sequence data from CRISPR-knockout cell lines that show a reduced indel frequency in MSH6- compared to MLH1-, MSH2- or PMS2-deficient cells. The redundancy for 1 bp indel repair between MSH2-MSH6 (MutSα) and MSH2-MSH3 (MutSβ) MMR heterodimers likely explains the reduced frequency of MNR variants in the constitutional tissues of MSH6-associated CMMRD. The inventors also observed genotype-phenotype correlations with respect to the type of MMR variant, with CMMRD patients carrying one or more MMR missense variants having lower MSI scores than those without. To the inventors knowledge, this is a novel observation for MMR genes and could have implications for our understanding of how MMR genotype influences mutation rate. It would be interesting, for example, to explore if MMR missense variants are associated with reduced MSI in MMR deficient tumours, and whether this has any association with clinical course. This strong genotype and MSI-burden correlations did not translate to differences in disease phenotype among the 56 CMMRD patients analysed, with no observed correlation of MMR genotype or MSI score with age of first tumour. Previously observed subtle differences in the incidence of CNS tumours and age of first tumour by affected MMR gene in CMMRD, but had analysed a larger cohort of 146 patients. In LS it is well established that the MMR genes are associated with distinct cancer spectra and risks. Previously it was also found both CRC and EC occurred earlier in carriers of PMS2 variants that cause loss of RNA expression compared to those that retain expression. However, there is otherwise very limited data supporting an effect for type or position of MMR variants on clinical phenotype. Regardless, it is likely disease phenotype correlates with MMR genotype in CMMRD but the association is far weaker than that between constitutional MSI-burden and MMR genotype.

The question then remains, why is there an apparent disconnect between constitutional MSI-burden and disease phenotype in CMMRD? There are several plausible explanations and a key limitation of our study is the restricted subgroup or multivariate analyses that might disentangle possible confounders due to cohort size. Constitutional MSI-burden is a combination of mutation rate and patient age at sampling. As age at sampling is positively correlated with age of first tumour, patients with less severe phenotypes will have had more time to accumulate microsatellite variants, as has been suggested for MSI in the general population and LS. Patient age, therefore, may confound associations between constitutional MSI-burden and disease penetrance. Analysing constitutional mutation rate directly would be superior, but would require alternative methods to quantify, for example, serial sampling of individuals or use of models, which have their own limitations. Furthermore, repair of microsatellite indels is only one of several functions of the MMR system within the DDR. In particular, both MMR-deficiency related single base substitutions (SBS) and indel mutations appear to drive MMR deficient tumorigenesis, and, whilst indel frequency was reduced, MSH6-deficient tissues have an equivalent increase in SBS frequency relative to MLH1-, MSH2-, and PMS2-deficient tissues in CRISPR knockouts of these genes. It is also possible that these mechanisms of mutation contribute to differing degrees in different tissues. For example, CMMRD brain and haematological malignancies have a reduced MSI signal compared to CMMRD LS-related carcinomas, increased MSI is common to LS-associated carcinomas but not brain or haematological malignancies in the sporadic population, and CMMRD brain tumours are typically ultra-hypermutated with >100 mutations/Mb associated with concurrent deficiencies of polymerase proofreading and MMR. Genetic and environmental backgrounds may determine the degree to which MSI or SBS contribute to tumorigenesis, with traditional PCR and fragment length analysis finding only 40% of gastrointestinal tumours to be MSI-H in CMMRD whilst >90% are in LS. The MMR system also signals to the wider DDR, for example to induce cell cycle arrest and apoptosis, and some MMR variants may promote tumorigenesis through these pathways rather than through, or in combination with, reduced repair capacity.

Environmental and genetic modifiers of cancer risk are also unaccounted for by the MSI score used in this study. Familial modifiers are to known to have large effects on cancer risk in LS and genetics may be of particular importance in CMMRD given parental consanguinity is seen in approximately half of CMMRD families. Familial risk factors might also explain the strong correlation in MSI score between patients sharing the same genotype observed in this study. With respect to tumorigenesis, this could imply that other factors have a more significant contribution to tumour initiation or progression than MMR deficiency, consistent with early models of LS colorectal tumorigenesis. We also explored whether tumour at the time of sampling is associated with MSI score, but no difference was found. It was interesting, however, that some CMMRD-negative and LS samples showed marginally increased MSI scores. Though beyond the scope of this study, further exploration of the effect of contaminating MSI-H circulating tumour cells on blood MSI analysis may be warranted.

In summary, we have analysed the constitutional MSI-burden of one of the largest cohorts of CMMRD patients in the scientific literature thus far, combining novel MSI markers and a simple method that could enhance CMMRD diagnostics. Our data show a strong association of constitutional MSI-burden with MMR genotype.

### Example 3—Development of Markers Optimised for Multiplex PCR

### Background

For MSI assay development, molecular inversion probes (MIPs) were used to facilitate robust multiplex amplification of MSI markers, and other genetic loci of clinical interest such as tumour mutation hotspots, without apparent limitation to the number of loci analysed. However, MIPs have limitations. In particular, MIPs require a minimum reaction input of ˜25 ng of sample DNA for reliable amplification. We have found that, in diagnostic practice at the Northern Genetics Service (Newcastle-upon-Tyne Hospitals NHS Foundation Trust), 14% of tumour DNA samples are of a quality/quantity too low to be analysed by MIPs and must be analysed by a “salvage” pathway. Furthermore, the MIP protocol is typically run over 2 days, which restricts sequencing to two batches per week with a median turnaround time of 10 days from sample receipt to report.

Multiplex amplification by traditional PCR methods is limited by primer-primer and primer-amplicon cross reactivity between target loci, and hence the number of loci that can be amplified is very variable (depending on loci and primer design, etc) and often limited to 10 loci or fewer. However, multiplex PCR can amplify from <1 ng of sample DNA, and its use instead of MIPs would, therefore, remove the need for a salvage pathway and streamline the diagnostic pipeline in practice. Multiplex PCR amplification also requires a shorter (≤1 day) protocol, which would allow 3 (or more) sequencing batches per week, increasing throughput and cutting overall turnaround time to ≤7 days from sample receipt to report.

The inventors have recently demonstrated that a two-round multiplex PCR assay of 12 of the best MSI markers described in WO/2018/037231 can be used to accurately test for MSI in resected tumour DNA samples as well as low quantity/quality samples, including genomic DNA extracted from endoscopic biopsies of colorectal cancers and cell free DNA extracted from urine (Phelps et al, doi: 10.3390/cancers14153838). Multiplex PCR can, therefore, provide an accurate alternative assay of our MSI markers that overcomes the limitations of MIPs.

The two-round multiplex PCR MSI assay described in Phelps et al 2022 had the potential to be simplified further to a single-round of multiplex PCR, which is an even shorter protocol. The published two-round multiplex PCR MSI assay also used in the inventors' original MSI markers (described WO/2018/037231), which the inventors have demonstrated are less sensitive to MMR deficiency in both blood and tumour tissues than the new MSI markers when analysed by MIP amplification and sequencing, as described herein. Therefore, the inventors developed an MSI assay using a single-round multiplex PCR assay of 14 MSI markers (3 original and 11 new), as well as BRAF and RAS mutation hotspots. It will be appreciated that the addition of mutation hotspots to the assay is optional. Furthermore, the selection of hotspots may depend upon the type of cancer investigated.

### Marker Selection

PCR primers for the 62 new MSI markers, described in Table A, were first designed and tested using the two-round multiplex PCR assay, which has a much lower setup cost than the single-round multiplex PCR method. PCR primer design followed the protocol of Phelps et al (2022). In brief, PCR primers were designed with 8N molecular barcodes (4N in each primer) using PCRTiler v1.42 with GrCH37/hg19 as reference and a melting temperature range of 57-61° C. Amplicon size was initially set at a maximum of 90 bp, and then increased by 10 bp incrementally if no usable primer pairs were obtained. Multiplex Manager was used to select primers which minimised primer interactions within the multiplex. Two-round multiplex PCR primer were successfully designed and produced amplicons in initial tests following the two-round multiplex PCR protocol (Phelps et al 2022) for 26 MSI markers (Table 4).

TABLE 4
Successful two-round multiplex PCR primer designs for new MSI markers. These
primers are used in the first round of PCR. Ns in the primer sequence represent molecular
barcodes. The common sequence 5′ of the molecular barcode (TCCGACGGTAGTGT for
forward primers, TCGGGAAGCTGAAG for reverse primers) act as annealing sites for the
universal amplification primers in the second PCR.

| MSI Marker | Primer Name | Primer Sequence (5′ to 3′) |
|---|---|---|
| EJmono12_SNP1 | EJ-M12_1F | TCCGACGGTAGTGTNNNNACTTGAGAAAGATGTGGTCAACT (SEQ |
| | | ID NO: 99) |
| | EJ-M12_1R | TCGGGAAGCTGAAGNNNNACAGGTTTACAAATTCCCACCT (SEQ ID |
| | | NO: 100) |
| MSJmono20_SNP1 | MSJ-M20_1F | TCCGACGGTAGTGTNNNNATTGTACTCCAGCACGGGT (SEQ ID NO: |
| | | 101) |
| | MSJ-M20_1R | TCGGGAAGCTGAAGNNNNATTTTACTTCCTCATTTTCCTGGAG (SEQ |
| | | ID NO: 102) |
| AKMmono07_SNP1 | AKM-M7_2F | TCCGACGGTAGTGTNNNNAAGGCCTTTTCCCTAAGGTG (SEQ ID NO: |
| | | 103) |
| | AKM-M7_2R | TCGGGAAGCTGAAGNNNNTGGGGCACTTAGATTTTACAGC (SEQ ID |
| | | NO: 104) |
| MSJmono45_SNP1 | MSJ_M45_2F | TCCGACGGTAGTGTNNNNTCATGCTACTGCACTCCAGC (SEQ ID NO: |
| | | 105) |
| | MSJ_M45_2R | TCGGGAAGCTGAAGNNNNGGAACAGACGGGCCAAACT (SEQ ID |
| | | NO: 106) |
| AKMmono05_SNP1 | AKM-M5_2F | TCCGACGGTAGTGTNNNNTGCTTTCTGATTGGTTCCCAAAA (SEQ ID |
| | | NO: 107) |
| | AKM-M5_2R | TCGGGAAGCTGAAGNNNNATGACAGGAATCCTCATATATCTTT (SEQ |
| | | ID NO: 108) |
| AKMmono02_SNP1 | AKM-M2_1F | TCCGACGGTAGTGTNNNNAACTCCTCTAAGGAATATGCTCTC (SEQ |
| | | ID NO: 109) |
| | AKM-M2_1R | TCGGGAAGCTGAAGNNNNACACACTAGTTGCATACATGCTT (SEQ ID |
| | | NO: 110) |
| AKMmono13_SNP1 | AKM13_1F | TCCGACGGTAGTGTNNNNAATAAAAACCACCACACAGAAGG (SEQ |
| | | ID NO: 111) |
| | AKM13_1R | TCGGGAAGCTGAAGNNNNGAGCTTGATTAAAGTCACAACATC (SEQ |
| | | ID NO: 112) |
| LMmono08_SNP1 | LM-M8_1F | TCCGACGGTAGTGTNNNNTGTGCTTGAGTTTGTTGAGC (SEQ ID NO: |
| | | 113) |
| | LM-M8_1R | TCGGGAAGCTGAAGNNNNGCTCCCAAAGGAGGAGAAGA (SEQ ID |
| | | NO: 114) |
| MSJmono39_SNP1 | MSJ-M39_1F | TCCGACGGTAGTGTNNNNACTTAGCTAGATAAAACACACATTT (SEQ |
| | | ID NO: 115) |
| | MSJ-M39_1R | TCGGGAAGCTGAAGNNNNCCATGTATCGTATTTACTGGTGGT (SEQ |
| | | ID NO: 116) |
| LMmono03_SNP1 | LM-M3_1F | TCCGACGGTAGTGTNNNNTAACACCTCAGTGGTAACTATTTT (SEQ |
| | | ID NO: 117) |
| | LM-M3_1R | TCGGGAAGCTGAAGNNNNCTTTTTCAGTTAGTATCACTAGGGC (SEQ |
| | | ID NO: 118) |
| AKMmono03_SNP1 | AKM-M3_1F | TCCGACGGTAGTGTNNNNGGCATTCAAGTCTAACAGAGCTTA (SEQ |
| | | ID NO: 119) |
| | AKM-M3_1R | TCGGGAAGCTGAAGNNNNTGATGCCCATCTTTCGTACTT (SEQ ID |
| | | NO: 120) |
| MSJmono46_SNP1 | MSJ-M46-1F | TCCGACGGTAGTGTNNNNTGAAGCTCCATTTATCTTTGGAC (SEQ ID |
| | | NO: 121) |
| | MSJ-M46-1R | TCGGGAAGCTGAAGNNNNTGTCTTACATAAATCTAAAACCATTG |
| | | (SEQ ID NO: 122) |
| AKMmono12_SNP1 | AKM-M12-F1 | TCCGACGGTAGTGTNNNNCCACTACTGTTTATGCAGCAAG (SEQ ID |
| | | NO: 123) |
| | AKM-M12-R1 | TCGGGAAGCTGAAGNNNNGCTGGAGTTTTCAAATTACACTG (SEQ |
| | | ID NO: 124) |
| AKMmono16_SNP1 | AKM-M16_1F | TCCGACGGTAGTGTNNNNATGCATTTGCTCCTTCACACTTG (SEQ ID |
| | | NO: 125) |
| | AKM-M16_1R | TCGGGAAGCTGAAGNNNNCACTGAGGCAGTATCTCTGTCAT (SEQ |
| | | ID NO: 126) |
| AKMmono04_SNP1 | AKM_M4_1F | TCCGACGGTAGTGTNNNNTGGACACTGACCAAATTGTCAAAC (SEQ |
| | | ID NO: 127) |
| | AKM_M4_1R | TCGGGAAGCTGAAGNNNNAAGCAATTAAACACACTTTTGGTG (SEQ |
| | | ID NO: 128) |
| MSJmono10_SNP1 | MSJ-M10-F1 | TCCGACGGTAGTGTNNNNTTCCTTTCCACATAAACAACCTT (SEQ ID |
| | | NO: 129) |
| | MSJ-M10-R1 | TCGGGAAGCTGAAGNNNNCTCCCCAAATTGATGTACAGGTTT (SEQ |
| | | ID NO: 130) |
| EJmono05_SNP1 | EJ-M5-F1 | TCCGACGGTAGTGTNNNNGAGGTTTCACTGCTGCTTTAGT (SEQ ID |
| | | NO: 131)_ |
| | EJ-M5-R1 | TCGGGAAGCTGAAGNNNNATGTGACTCAAGCTACCATCCT (SEQ ID |
| | | NO: 132) |
| LMmono12_SNP1 | LM-M12_1F | TCCGACGGTAGTGTNNNNGTGTCACTGTGGATAAAGACTAAGA |
| | | (SEQ ID NO: 133) |
| | LM-M12_1R | TCGGGAAGCTGAAGNNNNAGATGCACTTTGAAACAGCCTT (SEQ ID |
| | | NO: 134) |
| EJmono01_SNP1 | EJ-M1-F3 | TCCGACGGTAGTGTNNNNTATGTTTCTGTGCCTATATTCTGC (SEQ ID |
| | | NO: 135) |
| | EJ-M1-R3 | TCGGGAAGCTGAAGNNNNTGTCATTTTTGTCTTATTTCCTCAAA (SEQ |
| | | ID NO: 136) |
| EJmono06v2_SNP1 | EJ-M6_2F | TCCGACGGTAGTGTNNNNTTGCCCTTTCATTAGTGTGGAAA (SEQ ID |
| | | NO: 137) |
| | EJ-M6_2R | TCGGGAAGCTGAAGNNNNGCCTCAAGGGTTAGGCTG (SEQ ID NO: |
| | | 138) |
| EJmono02_SNP1 | EJ-M2_1F | TCCGACGGTAGTGTNNNNACCAAGAGACAGAATTCCACAT (SEQ ID |
| | | NO: 139) |
| | EJ-M2_1R | TCGGGAAGCTGAAGNNNNGCACAGGTGTCTTTGGTGAT (SEQ ID |
| | | NO: 140) |
| LMmono04v2_SNP1 | LM-M4-1F | TCCGACGGTAGTGTNNNNATGCCATGTTCCAATCAATTCTAAA (SEQ |
| | | ID NO: 141) |
| | LM-M4-1R | TCGGGAAGCTGAAGNNNNGTGGAATGAAGAAGCGATGTCA (SEQ |
| | | ID NO: 142) |
| LMmono07_SNP1 | LM-M7-1F | TCCGACGGTAGTGTNNNNATCTTGGACTTCCAAACCGCT (SEQ ID |
| | | NO: 143) |
| | LM-M7-1R | TCGGGAAGCTGAAGNNNNAGTTGAGACTGGGTACTTAAGAAAG |
| | | (SEQ ID NO: 144) |
| EJmono04_SNP1 | EJ_M4_1F | TCCGACGGTAGTGTNNNNGGTGAGCTCTAACCAAATCCA (SEQ ID |
| | | NO: 145) |
| | EJ_M4_1R | TCGGGAAGCTGAAGNNNNTGGGTAATTTTATTTGTGAATGGT (SEQ |
| | | ID NO: 146) |
| AKMmono06_SNP1 | AKM-M6_1F | TCCGACGGTAGTGTNNNNACATGGTCTTTGAAGATTATCCAAT (SEQ |
| | | ID NO: 147)_ |
| | AKM-M6_1R | TCGGGAAGCTGAAGNNNNGCAACCAAGTAGTTGAAGTTAGGT (SEQ |
| | | ID NO: 148) |
| AKMmono22_SNP1 | AKM-M22_1F | TCCGACGGTAGTGTNNNNCTTTGCCCCAGGGTAAAATTG (SEQ ID |
| | | NO: 149) |
| | AKM-M22_1R | TCGGGAAGCTGAAGNNNNGTCATCTCATCACATGAATAGGAA (SEQ |
| | | ID NO: 150) |

Sequencing of amplicons generated by two-round multiplex PCR amplification of different combinations of these 26 new MSI markers, along with original MSI markers and, optional BRAF and RAS mutation hotspots, identified a set of 19 new MSI markers that were most robust to multiplexing. It shall be appreciated by the skilled person that mixing lots of primers together in one reaction (i.e. a multiplex) may alter their performance compared to when primers are in singleplex. Therefore, the 26 markers that had initially been selected by singleplex analysis were then reduced to 19 based on performance in the two-round multiplex PCR method to see which primers worked best in a multiplex format. To do this, the inventors mixed the primers in different combinations, assessed them by gel electrophoresis as per the singleplex analysis, but also sequenced the amplicons to look at per marker read depths from the different primer combinations. The inventors selected MSI markers with highest read depths and that behaved most consistently across multiplexes.

These 19 robust new MSI markers, combined with the best 6 original MSI markers, were amplified by two-round multiplex PCR and sequenced from a cohort of 72 MSI-H and 72 MSS CRCs. The reference method for MSI status of samples was the MSI Analysis System v1.2 (Promega).

For each MSI marker, its ability to separate MSI-H from MSS CRCs was defined as the receiver operator characteristic area under curve (ROC AUC) calculated from sample reference allele frequencies (RAF, i.e. the proportion of reads containing the reference or wild type length of microsatellite). 16/19 of the new MSI markers and 4/6 original MSI markers achieved RAF ROC AUCs >0.95 using the two-round multiplex PCR assay (Table 5) demonstrating high accuracy for MSI detection using multiplex PCR.

TABLE 5
Reference allele frequency (RAF) receiver operator characteristic
area under curve (ROC AUC) values for the ability of MSI
markers (19 new, 6 original) to discriminate between 72
MSI-H and 72 MSS colorectal cancers (CRCs). MSI markers
were amplified and sequenced from CRCs by the two-round
multiplex PCR protocol described by Phelps et al (2022).

| Set | MSI Marker | RAF ROC AUC |
|---|---|---|
| New | EJmono12_SNP1 | 1.0000 |
| New | AKMmono12_SNP1 | 0.9987 |
| New | AKMmono07_SNP1 | 0.9987 |
| Original | LR44_SNP1 | 0.9982 |
| New | MSJmono20_SNP1 | 0.9982 |
| New | AKMmono13_SNP1 | 0.9969 |
| New | AKMmono05_SNP1 | 0.9969 |
| New | LMmono12_SNP1 | 0.9956 |
| Original | LR52_SNP1 | 0.9951 |
| New | AKMmono02_SNP1 | 0.9938 |
| New | AKMmono03_SNP1 | 0.9938 |
| New | MSJmono45_SNP1 | 0.9929 |
| New | LMmono03_SNP1 | 0.9911 |
| Original | LR36_SNP1 | 0.9792 |
| Original | GM07_SNP1 | 0.9734 |
| New | AKMmono16_SNP1 | 0.9707 |
| New | LMmono04_SNP1 | 0.9681 |
| New | AKMmono04_SNP1 | 0.9654 |
| New | EJmono01_SNP1 | 0.9535 |
| New | AKMmono22_SNP1 | 0.9508 |
| New | EJmono02_SNP1 | 0.9473 |
| New | LMmono07_SNP1 | 0.9419 |
| New | MSJmono39_SNP1 | 0.9406 |
| Original | GM11_SNP1 | 0.8927 |
| Original | GM14_SNP1 | 0.8865 |

The two-round multiplex PCR primers were redesigned to incorporate the universal amplification primers such that amplification could be completed in a single-round of multiplex PCR. The single-round multiplex PCR protocol is unpublished. In brief, each reaction contains 5 μl of 5×HS VeriFi Buffer (PCR Biosystems), 0.25 μl of 2 U/μl HS VeriFi DNA Polymerase (PCR Biosystems), 1 μl of multiplex primer mix with each primer at 1 μM in the stock, 1-5 μl of DNA sample, and molecular grade H₂O to achieve a total reaction volume of 25 μl. Reactions are incubated in a thermocycler using the following programme:

| | | | |
|---|---|---|---|
| Heat activation: | 95° C. | 1 | min |
| Amplification | 95° C. | 30 | sec |
| (30 cycles): | 57° C. | 90 | sec |
| | 72° C. | 60 | sec |
| Final extension: | 72° C. | 2 | min |

| | | |
|---|---|---|
| Hold: | 4° C. | ∞ |

Amplicon library preparation and sequencing follow established protocols (Phelps et al 2022).

Initial testing of primer multiplexes to amplify different combinations of MSI markers defined the final marker panel, containing 11 new and 3 original MSI markers (Table I), as well as 7 BRAF and RAS optional tumour mutation hotspots relevant to CRC care (not shown in Table I). In Table I, Primer Name, “xxx” is a unique sample index number and each primer must be purchased for each sample index. In Primer Sequence, the [Index8N] is the 8 base sequence of the sample index.

The finalised single-round multiplex PCR assay of 11 new and 3 original MSI markers, plus 7 BRAF and RAS mutation hotspots was subsequently validated using FFPE CRC DNA samples, NEQAS standards (https://ukneqas.org.uk/), and cancer cell lines. This included a training cohort of 50 MSI-H and 50 MSS CRCs to train the naïve Bayesian MSI classifier previously used for tumour analysis (Redford et al 2018, PLOS One 13(8):e0203052. doi: 10.1371/journal.pone.0203052, PMID: 30157243) and a validation cohort of 55 MSI-H and 83 MSS CRCs, as well as 4 MSI-H and 4 MSS NEQAS standards and 3 MSI-H and 3 MSS cancer cell lines. The CRC validation cohort deliberately contained samples of very low quantity and samples that had previously failed sequence analysis by MIPs to challenge the single-round multiplex PCR assay. The reference method for MSI status of samples was the MSI Analysis System v1.2 (Promega) or the MIP-based MSI assay (Gallon et al 2020, Human Mutation 41(1):332-341. doi: 10.1002/humu.23906, PMID: 31471937). Once trained, the naïve Bayesian MSI classifier generates an MSI score for each sample, with MSI scores >0 classifying the sample as MSI-H and MSI scores <0 classifying the sample as MSS.

Quality control (QC) thresholds were set for the single-round multiplex PCR MSI assay, requiring a median 100 reads for the MSI markers for a sample to pass QC. NEQAS standards and cancer cell lines all passed QC and were correctly classified (FIG. 27). 97 MSI-H and 110 MSS CRCs passed QC, and among these the MSI assay achieved 99.0% sensitivity (96/97) and 100.0% specificity (110/110) (FIG. 27). 8 MSI-H and 23 MSS CRCs from the validation cohort failed QC, with 6 of these having read depths too low to generate an MSI score. Despite this, the remaining 25 QC-fail CRCs were all correctly classified, although MSI scores clustered around 0 (an indeterminate score). Subsequently, this was demonstrated to be an issue of sample processing; nearly all of these samples came from a small number of DNA extraction batches and purification or dilution followed by a repeat assay improved QC metrics and MSI scores moved away from 0 (i.e. MSI-H CRC scores increased, and MSS CRC scores decreased, data not shown).

TABLE 6
examples of hotspots and associated primers suitable for a single-round multiplex
PCR reaction as described herein

| Set | Marker | Primer Name | Primer Sequence (5′ to 3′) |
|---|---|---|---|
| Hotspot | BRAF_ | BRAF_V600_SR_ | CAAGCAGAAGACGGCATACGAGAT[Index8N]ACACGCACGATCCGACGGT |
| | c1799T | SIxxxF | AGTGTCTGTTCAAACTGATGGGACC (SEQ ID NO: 85) |
| | | BRAF_V600_SR_ | AATGATACGGCGACCACCGAGATCTACACATACGAGATCCGTAATCGGGAA |
| | | UR | GCTGAAGCTTCATGAAGACCTCACAGTAAA (SEQ ID NO: 86) |
| Hotspot | KRASmr | KRAS_G12_SR_ | CAAGCAGAAGACGGCATACGAGAT[Index8N]ACACGCACGATCCGACGGT |
| | 1 | SIxxxF2 | AGTGTATTATAAGGCCTGCTGAAAATGACT (SEQ ID NO: 87) |
| | | KRAS_G12_SR_ | AATGATACGGCGACCACCGAGATCTACACATACGAGATCCGTAATCGGGAA |
| | | UR2 | GCTGAAGTAGCTGTATCGTCAAGGCACTCTT (SEQ ID NO: 88) |
| Hotspot | KRASmr | KRAS_Q61_SR_ | CAAGCAGAAGACGGCATACGAGAT[Index8N]ACACGCACGATCCGACGGT |
| | 2 | SIxxxF2 | AGTGTAGTCCTCATGTACTGGTCCCTC (SEQ ID NO: 89) |
| | | KRAS_Q61_SR_ | AATGATACGGCGACCACCGAGATCTACACATACGAGATCCGTAATCGGGAA |
| | | UR2 | GCTGAAGACCTGTCTCTTGGATATTCTCGAC (SEQ ID NO: 90) |
| Hotspot | KRASmr | KRAS_S145_SR_ | CAAGCAGAAGACGGCATACGAGAT[Index8N]ACACGCACGATCCGACGGT |
| | 3 | SIxxxF2 | AGTGTATTTCAGTGTTACTTACCTGTCTTG (SEQ ID NO: 91) |
| | | KRAS_S145_SR_ | AATGATACGGCGACCACCGAGATCTACACATACGAGATCCGTAATCGGGAA |
| | | UR2 | GCTGAAGACAGGCTCAGGACTTAGCAA (SEQ ID NO: 92) |
| Hotspot | NRASmr | NRAS_G12_SR_ | CAAGCAGAAGACGGCATACGAGAT[Index8N]ACACGCACGATCCGACGGT |
| | 1 | SIxxxF | AGTGTACAGGTTCTTGCTGGTGTG (SEQ ID NO: 93) |
| | | NRAS_G12_SR_ | AATGATACGGCGACCACCGAGATCTACACATACGAGATCCGTAATCGGGAA |
| | | UR | GCTGAAGACTGGGCCTCACCTCTATG (SEQ ID NO: 94) |
| Hotspot | NRASmr | NRAS_Q61_SR_ | CAAGCAGAAGACGGCATACGAGAT[Index8N]ACACGCACGATCCGACGGT |
| | 2 | SIxxxF2 | AGTGTTGTATTGGTCTCTCATGGCACT (SEQ ID NO: 95) |
| | | NRAS_Q61_SR_ | AATGATACGGCGACCACCGAGATCTACACATACGAGATCCGTAATCGGGAA |
| | | UR2 | GCTGAAGAGATGGTGAAACCTGTTTGTTGGA (SEQ ID NO: 96) |
| Hotspot | NRASmr | NRAS_S145_SR_ | CAAGCAGAAGACGGCATACGAGAT[Index8N]ACACGCACGATCCGACGGT |
| | 3 | SIxxxF2 | AGTGTATGCTGAAAGCTGTACCATACCT (SEQ ID NO: 97) |
| | | NRAS_S145_SR_ | AATGATACGGCGACCACCGAGATCTACACATACGAGATCCGTAATCGGGAA |
| | | UR2 | GCTGAAGACGAACTGGCCAAGAGTTACG (SEQ ID NO: 98) |

1. A method for evaluating levels of microsatellite instability in a sample, comprising:
a) analyzing the sample's DNA to determine the nucleotide sequence of one or more microsatellite marker, wherein the one or more microsatellite marker is selected from Table A; and
b) comparing the nucleotide sequence to a predetermined sequence, and determining any deviation, indicative of instability, from the predetermined sequences.

2. The method of claim 1, wherein the one or more microsatellite markers is 1, 2, 3, 4, 5, 6, 7, 8, 9, 10, 11, 12, 13, 14, 15, 16, 17, 18, 19, 20, 21, 22, 23, 24, or more, microsatellite markers selected from Table A.

3. The method of claim 2, wherein at least one of the microsatellite markers is selected from Table B or Table D, optionally wherein at least one of the markers is selected from the top 21 markers listed in Table B.

4. The method of claim 2, wherein at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or more microsatellite markers are selected from Table B or Table D, optionally wherein the at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or 21 microsatellite markers are selected from the top 21 markers listed in Table B.

5. The method of claim 1, wherein the one or more microsatellite markers selected from Table A is selected from the group of microsatellite markers listed in Table C.

6. The method of claim 5, wherein at least one of the microsatellite markers is selected from Table D.

7. The method of claim 5, wherein at least 2, at least 3, at least 4, least 5, least 6, least 7, least 8, least 9, least 10, least 11, least 12, least 13, least 14, least 15, least 16, least 17, least 18, least 19, least 20, least 21, least 22, least 23 or 24 microsatellite markers are selected from Table D.

8. The method of claim 1, wherein at least one of the markers is selected from the group consisting of AKMmono10v2, LMmono05v2, AKMmono05 and EJmono12 SNP1.

9. The method of claim 1, wherein the one or more microsatellite markers is 1, 2, 3, 4, 5, 6, 7, 8, 9, 10, 11, 12, 13, 14, 15, 16, 17, 18, 19, 20, 21, 22, 23, 24, 28 or more, microsatellite markers selected from Table H, optionally wherein the one or more microsatellite markers are the 32 markers listed in Table H.

10. The method of claim 1, wherein the one or more microsatellite markers is 1, 2, 3, 4, 5, 6, 7, 8, 9, 10, or 11 markers is selected from Table I.

11. The method of claim 10, wherein the method further comprises determining the nucleotide sequence of one or more microsatellite markers selected from Table G.

12. The method of claim 11, wherein the one or more microsatellite markers from Table G are LR36, GM07 and LR44.

13. The method of claim 10, wherein the method for comprises determining the nucleotide sequence of a cancer hotspot.

14. The method of claim 1, wherein the method comprises the step of amplifying from the sample one or more microsatellite marker selected from Table A to generate microsatellite markers amplicons prior to step a).

15. A method for evaluating the biological significance of sequence variation identified during sequencing, comprising:
a) amplifying from the sample one or more microsatellite marker selected from Table E to generate microsatellite markers amplicons, wherein each microsatellite loci has a single nucleotide polymorphism (SNP) within a short distance of the microsatellite marker and said amplifying step amplifies both the microsatellite marker and associated SNP in a single amplicon;
b) sequencing the amplicons; and
c) comparing the sequences from the amplicons to predetermined sequences and determining any deviation, indicative of instability, from the predetermined sequences; and
d) for heterozygous SNPs, determining whether there is a bias between indel frequencies for the two alleles.

16. The method of claim 15, wherein the one or more microsatellite markers is 1, 2, 3, 4, 5, 6, 7, 8, 9, 10, 11, 12, 13, or 14 microsatellite markers.

17. The method of claim 15, wherein the one or more markers selected from Table E may be AKMmono10v2 or LMmono05v2.

18. The method of claim 1, wherein the sample is a fluid sample or a solid sample, optionally wherein the fluid sample is a blood sample, urine sample, or part thereof.

19. The method of claim 18, wherein the part is peripheral blood leukocytes (PBLs).

20. The method of claim 1, wherein the subject has, is at risk of having, or is predisposed to a condition associated with microsatellite instability.

21. The method of claim 20, wherein the condition associated with microsatellite instability is cancer, CMMRD, Lynch syndrome, and/or Muir-Torre syndrome; preferably cancer or CMMRD.

22. The method of claim 21, wherein the cancer is selected from the group consisting of colon cancer, endometrium cancer, gastric cancer, ovarian cancer, hepatobiliary tract cancer, urinary tract cancer, stomach cancer, small intestine cancer, brain cancer, skin cancer, and haematological cancer.

23. A kit for amplifying one or more microsatellite marker selected from Table A, wherein the kit comprises primers and/or probes for specifically amplifying the one or more microsatellite marker.

24. A kit of claim 23, wherein the microsatellite marker is associated with a SNP and wherein the primers and/or probes are for specifically amplifying the one or more microsatellite marker and the associated SNP, optionally wherein the primers and/or probes have a sequence as shown in Table F, Table I, and/or Table 4.

25. (canceled)

26. (canceled)
